(19) United States
(12) Patent Application Publication
(10) Pub. No.: US 2017/0114025 A1
(43) Pub. Date: Apr. 27, 2017

(54) METHODS FOR INHIBITING NECROPTOSIS

(71) Applicant: Catalyst Therapeutics Pty Ltd, Parkville (AU)

(72) Inventors: Isabelle Lucet, Glen Waverley (AU); Jean-Marc Garnier, Albert Park (AU); James Michael Murphy, Yarraville (AU); John Hendry Silke, Travancore (AU); Joanne Maree Hildebrand, Watsonia North (AU); John Thomas Feutrill, Rosanna (AU); Anthony Nicholas Cuzzupe, St. Albans (AU); Guillaume Laurent Lessene, Bundoora (AU); Peter Edward Czabotar, North Melbourne (AU); Andrew Frederick Wilks, South Yarra (AU); Pooja Sharma, Pascoe Vale South (AU)

(21) Appl. No.: 15/311,020

(22) PCT Filed: May 15, 2015
(86) PCT No.: PCT/AU2015/050246

§ 371 Date: Nov. 14, 2016

(30) Foreign Application Priority Data
May 15, 2014 (AU) 2014901804
Sep. 8, 2014 (AU) 2014903569

(51) Int. Cl. C07D 239/48; C07D 213/74

(52) U.S. Cl. CPC C07D 239/48; C07D 213/74

(57) ABSTRACT

The present invention relates to methods for inhibiting necroptosis; screening methods for identifying compounds which inhibit necroptosis; and compounds for the inhibition of necroptosis, which may be useful in the treatment of conditions associated with deregulated necroptosis.

### FIELD OF THE INVENTION

The present disclosure relates to methods for inhibiting necroptosis, screening methods for identifying compounds which inhibit necroptosis and compounds for the inhibition of necroptosis.

### BACKGROUND OF THE INVENTION

In many diseases, cell death is mediated through apoptotic and/or necrotic pathways. While much is known about the mechanisms of action that control apoptosis, control of necrosis is not as well understood. Understanding the mechanisms regulating both necrosis and apoptosis in cells is essential to being able to treat conditions, such as neurodegenerative diseases, stroke, coronary heart disease, kidney disease, liver disease, AIDS and the conditions associated with AIDS.

Cell death has traditionally been categorized as either apoptotic or necrotic based on morphological characteristics (Wyllie et al., Int. Rev. Cytol. 68: 251 (1980)). These two modes of cell death were also initially thought to occur via regulated (caspase-dependent) and non-regulated processes, respectively. More recent studies, however, demonstrate that the underlying cell death mechanisms resulting in these two phenotypes are much more complicated and under some circumstances interrelated. Furthermore, conditions that lead to necrosis can occur by either regulated caspase-independent or non-regulated processes.

One regulated caspase-independent cell death pathway with morphological features resembling necrosis, called necroptosis, has been described (Degterev et al., Nat. Chem. Biol. 1:112, 2005). This manner of cell death can be initiated with various stimuli (e.g., TNF-[alpha] and Fas ligand) and in an array of cell types (e.g., monocytes, fibroblasts, lymphocytes, macrophages, epithelial cells and neurons). Necroptosis may represent a significant contributor to and in some cases predominant mode of cellular demise under pathological conditions involving excessive cell stress, rapid energy loss and massive oxidative species generation, where the highly energy-dependent apoptosis process is not operative.

The identification and optimization of low molecular weight molecules capable of inhibiting necroptosis will assist in elucidating its role in disease pathophysiology and could provide compounds for anti-necroptosis therapeutics. The discovery of compounds that prevent caspase-independent cell death (e.g., necrosis or necroptosis) would also provide useful therapeutic agents for treating or preventing conditions in which necrosis occurs. These compounds and methods would be particularly useful for the treatment of neurodegenerative diseases, ischemic brain and heart injuries, head trauma and inflammatory conditions.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

### SUMMARY OF THE INVENTION

In one aspect, there is provided a method for inhibiting necroptosis in a subject in need thereof, the method comprising administering a therapeutically effective amount of a compound that binds to the ATP-binding site of the pseudokinase domain of Mixed Lineage Kinase Domain-like (MLKL) protein.

In another aspect, there is provided a screening method for identifying a compound which inhibits necroptosis, the method comprising:
a) contacting a protein solution containing MLKL with a candidate compound under conditions allowing the interaction of MLKL and the candidate compound; and
b) comparing the unfolding transition temperature (Tₘ) obtained in the presence of the candidate compound with the unfolding transition temperature (Tₘ) obtained in the absence of the candidate compound to determine the change in the unfolding transition temperature (ΔTₘ);
wherein the interaction of MLKL and the candidate compound is through binding of the candidate compound to the ATP-binding site of the pseudokinase domain of MLKL; and
wherein a positive ΔTₙ value indicates that the candidate compound stabilizes the protein from denaturation and inhibits its role in necroptosis.

In another aspect, there is provided a screening method for identifying a compound which inhibits necroptosis, the method comprising:
a) contacting an MLKL pseudokinase domain with increasing concentrations of a candidate compound under conditions allowing the interaction of MLKL pseudokinase domain and the candidate compound; and
b) determining the binding affinity (K_{d});
wherein the interaction of the MLKL pseudokinase domain and the candidate compound is through binding of the candidate compound to the ATP-binding site of the pseudokinase domain of MLKL, and
wherein binding of the candidate compound indicates that the candidate compound is capable of inhibiting necroptosis.

In a further aspect, there is provided a screening method for identifying a compound which inhibits necroptosis, the method comprising:
a) contacting a protein solution containing MLKL pseudokinase domain with a nucleotide and a candidate compound and performing STD-NMR; and
b) comparing the STD-NMR spectrum obtained in the presence of the candidate compound and the STD-NMR spectrum obtained in the absence of the candidate compound;
wherein the interaction of the MLKL pseudokinase domain and the candidate compound is through binding of the candidate compound to the ATP-binding site of the pseudokinase domain of MLKL; and wherein the disappearance or reduction of the signal intensity in the STD-NMR spectrum indicates that the candidate compound is capable of inhibiting necroptosis.

Particular compounds described in US2005/0085637 have been found to be suitable for inhibiting necroptosis.

In one aspect, therefore, there is provided a method for inhibiting necroptosis in a subject in need thereof, the method comprising administering a therapeutically effective amount of a compound according to Formula (I):

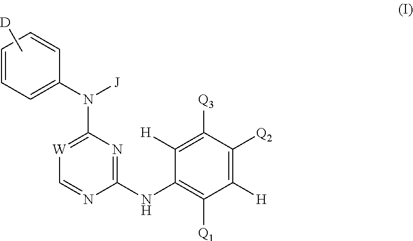

or a salt, solvate, or physiologically functional derivative thereof,

wherein:
W is N or C—R, wherein R is hydrogen, halogen, or cyano;
J is hydrogen, C₁-C₄ alkyl, C₁-C₄ haloalkyl, aralkyl, cyanoalkyl, (CH₂)ₚC═CH(CH₂)ₜH, —(CH₂)ₚC≡C(CH₂)ₜH, or C₃-C₇ cycloalkyl;
p is 1, 2, or 3;
t is 0 or 1;
D is —N(H)(X);
X is the group defined by —(X₁)—(X₂)_{q}—(X₃) wherein
X₁ is C(O) or C(S) and q is 1, or
X₁ is —C(O) or —S(O)₂ and q is 0,
X₂ is N(H) or O, and
X₃ is alkyl, cycloalkyl, heterocyclyl, alkoxy, aryloxy, aralkoxy, aryl, aralkyl, or heteroaryl, or
alkyl, cycloalkyl, heterocyclyl, alkoxy, aryloxy, aralkoxy, aryl, aralkyl, or heteroaryl substituted with at least one group defined by —(X₄)_{z}—(X₅),
   X₄ is C(H)₂ where z is 0, 1, 2, 3, or 4, and
   X₅ is hydrogen, C₁-C₆ alkyl, C₁-C₆ haloalkyl, cycloalkyl, heterocyclyl, aryl, heteroaryl, alkoxy, haloalkoxy, hydroxy, aryloxy, aralkoxy, halo, —CN, —NR′R′, N(H)C(O)R″, N(H)C(O)OR″, N(H)C(O)NR′R′, N(H)S(O)₂R″, OR″, OC(O)RR″, C(O)R″, SR″, —S(O)R′″, S(O)₂R′″,— or S(O)₂NR′R′, where,
   R′ is hydrogen, alkyl, cycloalkyl, heterocyclyl, —OR¹, —SR¹, —S(O)₂R¹, —S(O)R¹, or C(O)R¹;
   R′″ is hydrogen, alkyl, cycloalkyl, heterocyclyl, —OR¹, —NR³R⁴, —S(O)₂R¹, —S(O)R¹ or C(O)R¹; and
   R′″ is hydrogen, alkyl, cycloalkyl, heterocyclyl, —OR¹ or —NR³R⁴;
   Q₁ is hydrogen, halogen, C₁-C₂ haloalkyl, C₁-C₂ alkyl, C₁-C₂ alkoxy, or C₁-C₂ haloalkoxy;
   Q₂ is A¹ or A²;
   Q₃ is A¹ when Q₂ is A² and Q₃ is A² when Q₂ is A¹;
   wherein
   A¹ is hydrogen, halogen, C₁-C₃ alkyl, C₁-C₃ haloalkyl, —OR¹, and
   A² is the group defined by —(Z)ₘ—(Z¹)—(Z²), wherein
      Z is CH₂ and m is 0, 1, 2, or 3, or
      Z is NR² and m is 0 or 1, or
      Z is oxygen and m is 0 or 1, or
      Z is CH₂NR² and m is 0 or 1;
      Z¹ is S(O)₂, S(O), or C(O); and
      Z² is C₁-C₄ alkyl, cycloalkyl, heterocyclyl, NR³R⁴, aryl, arylamino, aralkyl, aralkoxy, or heteroaryl;
   R¹ is hydrogen, alkyl, heterocyclyl, and —NR³R⁴;
   R², R³, and R⁴ are each independently selected from hydrogen, hydroxy, alkoxy, aryloxy, aralkoxy, amino, alkylamino, arylamino, aralkylamino, C₁-C₄ alkyl, C₃-C₇ cycloalkyl, heterocyclyl, —S(O)₂R⁵, and —C(O)R⁵; and
      R⁵ is C₁-C₄ alkyl, or C₃-C₇ cycloalkyl.

In another aspect, there is provided a method for inhibiting necroptosis in a subject in need thereof, the method comprising administering a therapeutically effective amount of a compound according to Formula (II):

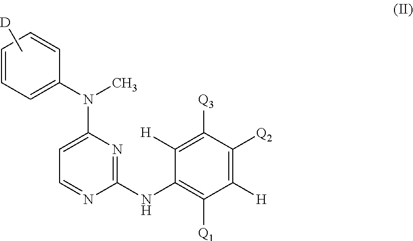

or a salt, solvate, or physiologically functional derivative thereof:
wherein:
   D is —N(H)(X);
   X is the group defined by —(X₁)—(X₂)_{q}—(X₃) wherein
   X₁ is C(O) or C(S) and q is 1, or
   X₁ is —C(O) or —S(O)₂ and q is 0,
   X₂ is N(H) or O, and
   X₃ is alkyl, cycloalkyl, heterocyclyl, alkoxy, aryloxy, aralkoxy, aryl, aralkyl, or heteroaryl, or
   alkyl, cycloalkyl, heterocyclyl, alkoxy, aryloxy, aralkoxy, aryl, aralkyl, or heteroaryl substituted with at least one group defined by —(X₄)_{z}—(X₅),
   X₄ is C(H)₂ where z is 0, 1, 2, 3, or 4, and
   X₅ is hydrogen, C₁-C₆ alkyl, C₁-C₆ haloalkyl, cycloalkyl, heterocyclyl, aryl, heteroaryl, alkoxy, haloalkoxy, hydroxy, aryloxy, aralkoxy, halo, —CN, —NR′R′, N(H)C(O)R″, N(H)C(O)OR″, N(H)C(O)NR′R′, N(H)S(O)₂R″, OR″, OC(O)R″, C(O)R″, SR″, —S(O)R′″, —S(O)₂R′″, or —S(O)₂NR′R′, where,
   R′ is hydrogen, alkyl, cycloalkyl, heterocyclyl, —OR¹, —SR¹, —S(O)₂R¹, —S(O)R¹, or C(O)R¹;
   R″ is hydrogen, alkyl, cycloalkyl, heterocyclyl, —OR¹, —NR³R⁴, —S(O)₂R¹, —S(O)R¹, or C(O)R¹; and
   R′″ is hydrogen, alkyl, cycloalkyl, heterocyclyl, —OR¹, or —NR³R⁴;
   Q₁ is hydrogen, halogen, C₁-C₂ haloalkyl, C₁-C₂alkyl, C₁-C₂ alkoxy, or C₁-C₂ haloalkoxy;
   Q₂ is A¹ or A²;
   Q₃ is A¹ when Q₂ is A² and Q₃ is A² when Q₂ is A¹;
   wherein
      A¹ is hydrogen, halogen, C₁-C₃ alkyl, C₁-C₃ haloalkyl, —OR, and
      A² is the group defined by —(Z)ₘ—(Z¹)—(Z²),
   wherein
      Z is CH₂ and m is 0, 1, 2, or 3, or
      Z is NR² and m is 0 or 1, or
      Z is oxygen and m is 0 or 1, or
      Z is CH₂NR² and m is 0 or 1;
      Z¹ is S(O)₂, S(O), or C(O); and
      Z² is C₁-C₄ alkyl, cycloalkyl, heterocyclyl, NR³R⁴, aryl, arylamino, aralkyl, aralkoxy, or heteroaryl;
   R¹ is hydrogen, heterocyclyl, and —NR³R⁴;
   R², R³, and R⁴ are each independently selected from hydrogen, hydroxy, alkoxy, aryloxy, aralkoxy, amino, alkylamino, arylamino, aralkylamino, C₁-C₄ alkyl, C₃-C₇ cycloalkyl, heterocyclyl, —S(O)₂R⁵, and —C(O)R⁵; and
R⁵ is C₁-C₄ alkyl, or C₃-C₇ cycloalkyl.

In another aspect, there is provided a method for inhibiting necroptosis in a subject in need thereof, the method comprising administering a therapeutically effective amount of a compound according to Formula (I):

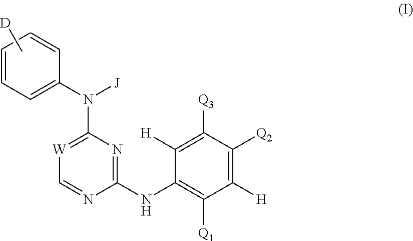

or a salt, solvate, or physiologically functional derivative thereof:

wherein:
W is N or C—R, wherein R is hydrogen, halogen, or cyano;
J is hydrogen, C₁-C₄alkyl, C₁-C₄ haloalkyl, aralkyl, cyanoalkyl, —(CH₂)ₚC═CH(CH₂)ₜH, —(CH₂)ₚC≡C(CH₂)ₜH, or C₃-C₇ cycloalkyl;
p is 1, 2, or 3;
t is 0 or 1;
D is

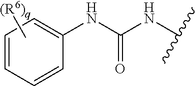

q is 1, 2, or 3;
Q₁ is hydrogen, halogen, C₁-C₂ haloalkyl, C₁-C₂ alkyl, C₁-C₂ alkoxy, or C₁-C₂ haloalkoxy;
Q₂ is A¹ or A²;
Q₃ is A¹ when Q₂ is A² and Q₃ is A² when Q₂ is A¹;
wherein
   A¹ is hydrogen, halogen, C₁-C₃ alkyl, C₁-C₃ haloalkyl, —OR^{1′} and
   A² is the group defined by —(Z)ₘ—(Z¹)—(Z²),
wherein
   Z is CH₂ and m is 0, 1, 2, or 3, or
   Z is NR² and m is 0 or 1, or
   Z is O and m is 0 or 1, or
   Z is CH₂NR² and m is 0 or 1;
   Z¹ is S(O)₂, S(O), or C(O); and
   Z² is C₁-C₄ alkyl, cycloalkyl, heterocyclyl, NR³R⁴, aryl, arylamino, aralkyl, aralkoxy, or heteroaryl;
      R², R³, and R⁴ are each independently selected from hydrogen, hydroxy, alkoxy, aryloxy, aralkoxy, amino, alkylamino, arylamino, aralkylamino, C₁-C₄ alkyl, C₃-C₇ cycloalkyl, heterocyclyl, —S(O)₂R⁵, and —C(O)R⁵;
      R⁵ is C₁-C₆alkyl, or C₃-C₇ cycloalkyl; and
      R⁶ is the group defined by —(X₄)_{z}—(X₅),
      wherein
         X₄ is C(H)₂ where z is 0, 1, 2, 3, or 4, and
         X₅ is hydrogen, C₁-C₆ alkyl, C₁-C₆ haloalkyl, cycloalkyl, heterocyclyl, aryl, heteroaryl, alkoxy, haloalkoxy, hydroxy, aryloxy, aralkoxy, halo, CN, —NR⁷R⁷, —N(H)C(O)R⁷, —N(H)C(O)ORR⁷R⁷N(H)S(O)₂R⁷, N(H)S(O)₂NR⁷R⁷, —OC(O)R⁷, OC(O)NR⁷R⁷, —C(O)R⁷, —C(O)NR⁷R⁷, —SR⁷, —S(O)R⁷, —S(O)₂R⁷R⁷, or —S(O)₂NR⁷R⁷; and
      R⁷ is hydrogen, C₁-C₆ alkyl, C₁-C₆ haloalkyl, cycloalkyl, heterocyclyl, alkylamino.

In another aspect, there is provided a method for inhibiting necroptosis in a subject in need thereof, the method comprising administering a therapeutically effective amount of a compound according to Formula (II):

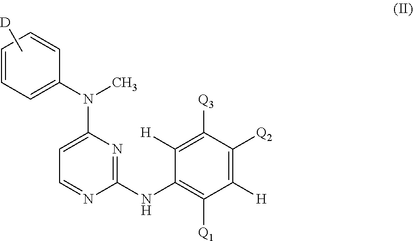

or a salt, solvate, or physiologically functional derivative thereof:

wherein:
D is

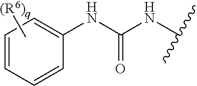

q is 1, 2, or 3;
Q₁ is hydrogen, halogen, C₁-C₂ haloalkyl, C₁-C₂ alkyl, C₁-C₂ alkoxy, or C₁-C₂ haloalkoxy;
Q₂ is A¹ or A²;
Q₃ is A when Q₂ is A² and Q₃ is A² when Q₂ is A¹;
wherein
   A¹ is hydrogen, halogen, C₁-C₃ alkyl, C₁-C₃ haloalkyl, —OR¹, and
   A² is the group defined by —(Z)ₘ—(Z¹)—(Z²),
wherein
   Z is CH₂ and m is 0, 1, 2, or 3, or
   Z is NR² and m is 0 or 1, or
   Z is O and m is 0 or 1, or
   Z is CH₂NR² and m is 0 or 1;
   Z¹ is S(O)₂, S(O), or C(O); and
   Z² is C₁-C₄ alkyl, cycloalkyl, heterocyclyl, NR³R⁴, aryl, arylamino, aralkyl, aralkoxy, or heteroaryl;
      R², R³, and R⁴ are each independently selected from hydrogen, hydroxy, alkoxy, aryloxy, aralkoxy, amino, alkylamino, arylamino, aralkylamino, C₁-C₄ alkyl, C₃-C₇ cycloalkyl, heterocyclyl, —S(O)₂RS, and —C(O)R⁵;
      R⁵ is C₁-C₆alkyl, or C₃-C₇ cycloalkyl; and
      R⁶ is the group defined by —(X₄)_{z}—(X₅), wherein
         X₄ is C(H)₂ where z is 0, 1, 2, 3, or 4, and
         X₅ is hydrogen, C₁-C₆ alkyl, C₁-C₆ haloalkyl, cycloalkyl, heterocyclyl, aryl, heteroaryl, alkoxy, haloalkoxy, hydroxy, aryloxy, aralkoxy, halo, CN, —NR⁷R⁷, —N(H)C(O)R⁷, —N(H)C(O)OR⁷, —N(H)C(O)NR⁷R⁷, N(H)S(O)₂R⁷, N(H)S(O)₂NR⁷R⁷, —OC(O)R⁷, OC(O)NR⁷R⁷, —C(O)R⁷, —O)NR⁷R⁷, —SR⁷, —S(O)R⁷, —S(O)₂R⁷R⁷, or —S(O)₂NR⁷R⁷; and
         R⁷ is hydrogen, C₁-C₆ alkyl, C₁-C₆ haloalkyl, cycloalkyl, heterocyclyl, alkylamino, alkoxy, aryloxy, aralkoxy, arylamino, aralkylamino, aryl or heteroaryl.

In another aspect, there is provided a method for inhibiting necroptosis in a subject in need thereof, the method comprising administering a therapeutically effective amount of a compound according to Formula (I):

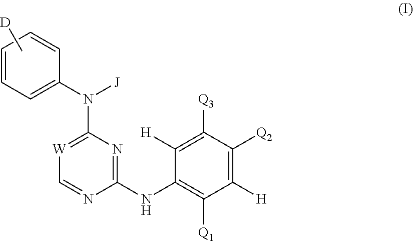

or a salt, solvate, or physiologically functional derivative thereof:

wherein:
W is N or C—R, wherein R is hydrogen, halogen, or cyano;
J is hydrogen, C₁-C₄alkyl, C₁-C₄ haloalkyl, aralkyl, cyanoalkyl, —(CH₂)ₚC═CH(CH₂)ₜH, —(CH₂)ₚC═C(CH₂)ₜH, or C₃-C₇ cycloalkyl;
p is 1, 2, or 3;
t is 0 or 1;
D is —N(R⁸)(X);
X is the group defined by —(X₁)—(X₂)_{q}—(X₃)

wherein
X₁ is C(O) or C(S) and q is 1, or
X₁ is —C(O) or —S(O)₂ and q is 0,
X₂ is N(H) or O, and
X₃ is alkyl, cycloalkyl, heterocyclyl, alkoxy, aryloxy, aralkoxy, aryl, aralkyl, or heteroaryl, or
alkyl, cycloalkyl, heterocyclyl, alkoxy, aryloxy, aralkoxy, aryl, aralkyl, or heteroaryl substituted with at least one group defined by —(X₄)_{z}—(X₅),
X₄ is C(H)₂ where z is 0, 1, 2, 3, or 4, and
X₅ is hydrogen, C₁-C₆ alkyl, C₁-C₆ haloalkyl, cycloalkyl, heterocyclyl, aryl, heteroaryl, alkoxy, haloalkoxy, hydroxy, aryloxy, aralkoxy, halo, —CN, —NR′R′, N(H)C(O)R″, N(H)C(O)OR″, N(H)C(O)NR′R′, N(H)S(O)₂R″, OR″, OC(O)R″, C(O)R″, SR″, —S(O)R′″, —S(O)₂R′″, or —S(O)₂NR′R′,
where
   R′ is hydrogen, alkyl, cycloalkyl, heterocyclyl, —OR, —SR¹, —S(O)₂R¹, —S(O)R¹, or C(O)R¹;
   R″ is hydrogen, alkyl, cycloalkyl, heterocyclyl, —OR¹, —NR³R⁴, —S(O)₂R¹, —S(O)R¹, or C(O)R¹; and
   R′″ is hydrogen, alkyl, cycloalkyl, heterocyclyl, —OR¹, or —NR³R⁴;
Q₁ is hydrogen, halogen, C₁-C₂ haloalkyl, C₁-C₂ alkyl, C₁-C₂ alkoxy, or C₁-C₂ haloalkoxy;
Q₂ is A¹ or A²;
Q₃ is A¹ when Q₂ is A² and Q₃ is A² when Q₂ is A¹;
wherein
   A¹ is hydrogen, halogen, C₁-C₃alkyl, C₁-C₃ haloalkyl, —OR¹, and
   A² is the group defined by —(Z)ₘ—(Z¹)—(Z²),
wherein
   Z is CH₂ and m is 0, 1, 2, or 3, or
   Z is NR² and m is 0 or 1, or
   Z is oxygen and m is 0 or 1, or
   Z is CH₂NR² and m is 0 or 1;
   Z¹ is S(O)₂, S(O), or C(O); and
   Z² is C₁-C₄ alkyl, cycloalkyl, heterocyclyl, NR³R⁴, aryl, arylamino, aralkyl, aralkoxy, or heteroaryl;
R¹ is hydrogen, alkyl, heterocyclyl, and —NR³R⁴;
R², R³, and R⁴ are each independently selected from hydrogen, hydroxy, alkoxy, aryloxy, aralkoxy, amino, alkylamino, arylamino, aralkylamino, C₁-C₄ alkyl, C₃-C₇ cycloalkyl, heterocyclyl, —S(O)₂R⁵, and —C(O)R⁵;
   R⁵ is C₁-C₄ alkyl, or C₃-C₇ cycloalkyl; and
   R⁸ is hydrogen or C₁-C₃ alkyl.

In another aspect, there is further provided novel compounds of Formulas (I), (II) and/or (III).

In another aspect, there is provided use of a compound of Formula (I), (II) and/or (III) in the preparation of a medicament for the inhibition of necroptosis in a subject.

In another aspect, there is provided use of a compound according to Formula (I), (II) and/or (III) for inhibiting necroptosis.

In yet another aspect, there is provided a compound according to Formula (I), (II) and/or (III) for use in inhibiting necroptosis.

In yet another aspect, there is provided a compound according to Formula (I), (II) and/or (III) when used for inhibiting necroptosis.

Any embodiment herein shall be taken to apply mutatis mutandis to any other embodiment unless specifically stated otherwise.

The present disclosure is not to be limited in scope by the specific embodiments described herein, which are intended for the purpose of exemplification only. Functionally-equivalent products, compositions and methods are clearly within the scope of the invention, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

The disclosure is hereinafter described by way of the following non-limiting Examples and with reference to the accompanying figures.

### BRIEF DESCRIPTION OF THE DRAWINGS

FIG. 1: Mechanistic studies of compound 1 inhibition of MLKL-induced necroptosis. (A): Compound 1 was identified as a mouse MLKL interactor using a thermal stability shift assay. (B): Compound 1 binding to the mouse MLKL pseudokinase domain was validated by Surface Plasmon Resonance (SPR). (C): Compound 1 inhibited necroptotic death of wild type MDFs stimulated with TSQ in a dose dependent manner. Data shown are the mean±SEM for 3 independent experiments. (D): Compound 1 retarded translocation to the membrane fraction in anti-MLKL blots of Blue-Native PAGE. Cytoplasmic and membrane fraction purity and protein abundance are illustrated by control blots for GADPH and VDAC1.

FIG. 2: (A): Saturation difference transfer (STD) NMR spectra showing nucleotide binding to mouse MLKL. The data show that compound 1 can compete with (i) ATP and (ii) ADP for binding to mouse MLKL pseudokinase domain. The low field region of the off resonance spectrum shows peaks detected for 200 μM ATP (i) or ADP (ii) in the absence of protein. Peaks marked with asterisks were observed in STD-NMR experiments performed on ATP (i) or ADP (ii) in the presence of 2 μM mouse MLKL(179-464), confirming nucleotide binding. These peaks were diminished in the presence of 200 μM compound 1, confirming that ATP and ADP and compound 1 are displaced from mouse MLKL(179-464) by addition of compound 1. (B): Toxicity of compound 1 induces death of wild-type MDFs at concentrations ≧5 μM. Mean±SEM of triplicate experiments shown. (C): In vitro kinase assay demonstrating that compound 1 has no impact on recombinant RIPK3 kinase activity relative to a DMSO control (“0” lanes). Compound 1 concentrations ≧10 μM reproducibly led to enhanced phosphorylation of mouse MLKL(179-464). Experiment shown is representative of three independent assays. Left panel, dried Coomassie stained 4-12% Bis-Tris gel; right panel, autoradiograph of the same gel.

FIG. 3: (A): STD NMR spectra showing nucleotide binding to human MLKL. The data show that compound 1 can compete with (i) ATP and (ii) ADP for binding to human MLKL pseudokinase domain. The low field region of the off resonance spectrum shows peaks detected for 200 μM ATP (i) or ADP (ii) in the absence of protein. Peaks marked with asterisks were observed in STD-NMR experiments performed on ATP (i) or ADP (ii) in the presence of 2 μM human MLKL(190-471), confirming nucleotide binding. These peaks were diminished in the presence of 200 μM compound 1, confirming that ATP and ADP and compound 1 are displaced from human MLKL(190-471) by addition of compound 1. (B): Compound 1 binding to human MLKL(190-471) was validated by SPR. (C): Compound 1 inhibited necroptotic death of the wild type U937 cell line stimulated with TSQ in a dose dependent manner. Data shown are the mean±SD for 5 independent experiments. (D): Toxicity of compound 1 in U937 cells gene-edited to delete MLKL. In the absence of MLKL, TSQ stimulated cells behaved equivalently to unstimulated cells. TS treatment illustrates that the cells have retained the capacity to undergo apoptotic death and this was unaffected by Compound 1 treatment. Data shown are mean±SD for 2 independent experiments.

FIG. 4: (A): Sorafenib, a protein kinase inhibitor with a similar protein kinase target profile to compound 1, did not inhibit TSQ-induced cell death in wild type MDFs at same or less than 1 μM concentration. Mean±SEM of triplicate experiments shown. (B): Sorafenib, a protein kinase inhibitor with a similar protein kinase target profile to compound 1, did not inhibit TSQ-induced necroptosis in wild type U937 cells. Mean±SEM of duplicate experiments shown.

FIG. 5: (A) and (B): Complex between compound 3 and human MLKL demonstrating that compound 3 binds within the ATP-binding site of human MLKL.

FIG. 6: Graph showing compound 1 inhibiting Poly(I:C)-induced RIP1-independent necroptosis.

FIG. 7: SEQ ID NO: 1 is an amino acid sequence of the human full length isoform of the MLKL protein.

FIG. 8: SEQ ID NO: 2 is an amino acid sequence of the human short isoform of the MLKL protein.

FIG. 9: SEQ ID NO: 3 is an amino acid sequence of the mouse full length isoform of the MLKL protein.

FIG. 10: SEQ ID NO: 4 is an amino acid sequence of the mouse short isoform of a 464-amino-acid MLKL protein.

### KEY TO SEQUENCE LISTING

SEQ ID NO: 1 is an amino acid sequence of the human full length isoform of the MLKL protein.

SEQ ID NO: 2 is an amino acid sequence of the human short isoform of the MLKL protein.

SEQ ID NO: 3 is an amino acid sequence of the mouse full length isoform of the MLKL protein.

SEQ ID NO: 4 is an amino acid sequence of the mouse short isoform of a 464-amino-acid MLKL protein.

### DETAILED DESCRIPTION OF THE EMBODIMENTS

It will be understood that the invention disclosed and defined in this specification extends to all alternative combinations of two or more of the individual features mentioned or evident from the text or drawings. All of these different combinations constitute various alternative aspects of the invention.

Programmed necrosis or “necroptosis” has emerged in the past 5 years as a cell death mechanism that complements the conventional cell death pathway, apoptosis, in multicellular organisms. In contrast to apoptosis, necroptosis does not appear to serve an important role in multicellular organism development, but plays a role in the defence against pathogens and is a likely culprit in many destructive inflammatory conditions. Receptor Interacting Protein Kinase-3 (RIPK3) was identified as a key effector of necroptosis in 2009, and its substrate, the pseudokinase Mixed Lineage Kinase Domain-Like (MLKL) in 2012, but the molecular events following RIPK3-mediated phosphorylation of MLKL required to induce cell death are unclear. The RIPK1/RIPK3/MLKL necrosome has been claimed to activate PGAM5 and Drp1 to cause mitochondrial fragmentation and cell death but the requirement for PGAM5, Drp1 and mitochondria for necroptosis has been questioned. MLKL is an essential effector protein in the necroptotic cell death pathway (Sun et al., Cell, 148(1-2), 213-227, 2012; Zhao et al. PNAS, 109(14), 5322-5327; Murphy et al. Immunity, 39(3), 443-453, 2013).

MLKL contains a C-terminal pseudokinase domain and an N-terminal four-helix bundle (4HB) domain connected by a two helix linker (the “brace” helices). The present disclosure is based on our mutational and biochemical analyses which demonstrate that the MLKL 4HB domain is sufficient to induce necroptosis, with several charged residues clustered on two faces of the 4HB domain being required for this function. Surprisingly, the polarity of several of these charged residues is not conserved between mouse and human MLKL and alanine substitution of negatively charged residues on the α4 Helix of the 4HB domain disrupted function. This finding challenges the importance of phospholipid binding to the killing activity of the 4HB domain and illustrates that membrane association cannot solely be attributed to the interaction of poorly conserved basic residues within the MLKL 4HB domain. Intriguingly, mutation of a second cluster of residues on the 4HB domain did not preclude oligomerization and membrane localization, illustrating that while membrane translocation is likely to underlie the killing function the 4HB domain, it is insufficient to induce cell death.

Without wishing to be bound by theory, these data support a model for MLKL function whereby the pseudokinase domain of MLKL holds the 4HB domain in check until phosphorylated by RIPK3, which causes a conformational change in the pseudokinase domain to release the 4HB domain to oligomerize and associate with membranes. This step in the activation of MLKL can be thwarted by a small molecule that targets the ATP-binding site within the MLKL pseudokinase domain and thereby retards MLKL translocation to membranes and prevents necroptosis. Targeting the ATP-binding site or “pseudoactive” site of pseudokinases, is a hitherto unexplored class of therapeutic targets. Accordingly, the identification of compounds which prevent necroptosis by targeting MLKL would be advantageous in the treatment of conditions associated with deregulated necroptosis.

The present disclosure demonstrates that MLKL oligomerization and membrane translocation can be inhibited by a compound, which was identified on the basis of its affinity for the ATP-binding site of the MLKL pseudokinase domain. Furthermore, the present disclosure demonstrates that inhibition of MLKL oligomerization and membrane translocation by a small molecule provides for the inhibition of necroptosis.

In one aspect of the present disclosure, there is provided a method for inhibiting necroptosis in a subject in need thereof, the method comprising administering a therapeutically effective amount of a compound that binds to the ATP-binding site of the pseudokinase domain of Mixed Lineage Kinase Domain-like (MLKL) protein.

As described hereinbefore, it is considered that the pseudokinase domain of MLKL holds the 4HB domain in check until phosphorylated by RIPK3, which causes a conformational change in the pseudokinase domain to release the 4HB domain to oligomerize and associate with membranes thereby resulting in necroptosis. Accordingly, in one embodiment of the present disclosure, administration of the compound inhibits a conformational change of MLKL. In another embodiment, the conformational change of MLKL involves release of the four-helix bundle (4HB) domain of MLKL. In another embodiment, administration of the compound inhibits oligomerisation of MLKL. In yet another embodiment, administration of the compound inhibits translocation of MLKL to the cell membrane. In a further embodiment, administration of the compound inhibits a conformational change of MLKL, inhibits oligomerisation of MLKL and inhibits translocation of MLKL to the cell membrane.

Both human and mouse MLKL have two transcript isoforms produced by alternative splicing. Human MLKL isoform 1 is the longer human transcript and encodes the full length isoform of a 471-amino-acid protein (SEQ ID NO: 1). Human isoform 2 lacks exons 4-8 and encodes the short isoform of a 263-amino-acid protein (SEQ ID NO: 2). Although the two isoforms of human MLKL have the same N- and C termini (as described in WO2010/122135), the full length human MLKL, but not the shorter isoform of the gene, has one protein kinase-like domain (as described in WO2010/122135; 190-471 of human MLKL (SEQ ID NO:1); Murphy et al. Biochem J 457(3):369-77, 2014). Both isoforms of mouse MLKL contain one protein kinase-like domain (amino acids 179-472 (SEQ ID NO: 3), 179-464 (SEQ ID NO: 4)). Accordingly, the compounds encompassed by the present disclosure bind to human MLKL isoform 1 (SEQ ID NO: 1), mouse MLKL isoforms 1 (SEQ ID NO: 3) and 2 (SEQ ID NO: 4) and various known natural variants thereof. Within the protein kinase-like domain of human MLKL lies an ATP-binding site (amino acids 209-217 of SEQ ID NO: 1) which binds ATP and other nucleotides including, but not limited to, AMP, ADP and AMPPNP. In human MLKL, ATP (and other nucleotides) is bound by a pocket of discontinuous residues which includes K230 from the β3 strand of the N-lobe and K331 from the counterpart of the “catalytic” loop of conventional protein kinases (Murphy et al. Biochem J 457(3):369-77, 2014). MLKL also has an N-terminal four helix bundle (4HB) domain (within amino acids 1-125 of SEQ ID NO: 1, 2, 3, 4; Murphy et al. Immunity, 39(3), 443-453, 2013). 4HB is a death effector domain within MLKL with the cell killing function of MLKL relying on the oligomerization and plasma membrane association of 4HB.

It is envisaged that compounds of the present disclosure can bind to MLKL in various species and inhibit necroptosis. For example, compounds of the present disclosure can bind to human MLKL (SEQ ID NO: 1 or variants or analogues thereof; Murphy et al. The Biochemical Journal, 457(3), 369-377, 2014) and inhibit necroptosis. In another example, compounds of the present disclosure can bind to mouse MLKL (SEQ ID NO: 3; SEQ ID NO: 4 or variants or analogues thereof; Murphy et al. Immunity, 39(3), 443-453, 2013) and inhibit necroptosis.

As used herein, the term “pseudokinase domain” as understood by a person skilled in the art, means a protein containing a catalytically-inactive or catalytically-defective kinase domain. “Pseudokinase domains” are often referred to as “protein kinase-like domains” as these domains lack conserved residues known to catalyse phosphoryl transfer. It would be understood by a person skilled in the art that although pseudokinase domains are predicted to function principally as catalysis independent protein-interaction modules, several pseudokinase domains have been attributed unexpected catalytic functions. Accordingly, in the present disclosure the term “pseudokinase domain” includes “pseudokinase domains” which lack kinase activity and “pseudokinase domains” which possess weak kinase activity.

As used herein, the term “ATP-binding site” as understood by a person skilled in the art, means a specific sequence of protein subunits that promotes the attachment of ATP to a target protein. An ATP binding site is a protein micro-environment where ATP is captured and hydrolyzed to ADP, thereby releasing energy that is utilized by the protein to work by changing the protein shape and/or making the enzyme catalytically active. In pseudokinase domains, the “ATP-binding site” is often referred to as the “pseudoactive site”. The term “ATP-binding site” may also be referred to as a “nucleotide-binding site” as binding at this site includes the binding of nucleotides other than ATP. It would be understood by a person skilled in the art that the term “nucleotide” includes any nucleotide. Exemplary nucleotides include, but are not limited to, AMP, ADP, ATP, AMPPNP, GTP, CTP and UTP.

As described herein, inhibition of necroptosis includes both complete and partial inhibition of necroptosis. In one embodiment, inhibition of necroptosis is complete inhibition. In another embodiment, inhibition of necroptosis is partial inhibition.

The binding of a compound to the ATP-binding site of the pseudokinase domain of MLKL may be determined by any method considered to be suitable by a person skilled in the art for such a use. In one embodiment of the present disclosure, the binding of a compound to the ATP-binding site of the pseudokinase domain of MLKL is determined by one or more assays selected from the group comprising, but not limited to, thermal shift assay, surface plasmon resonance (SPR), and saturation transfer difference NMR (STD-NMR). In another embodiment, the binding of a compound to the ATP-binding site of the pseudokinase domain of MLKL is determined by thermal shift assay. In yet another embodiment, the binding of a compound to the ATP-binding site of the pseudokinase domain of MLKL is determined by SPR. In yet another embodiment, the binding of a compound to the ATP-binding site of the pseudokinase domain of MLKL is determined by STD-NMR. In a further embodiment, the binding of a compound to the ATP-binding site of the pseudokinase domain of MLKL is determined by thermal shift assay and one or more additional assays. In yet a further embodiment, the additional assays are selected from the group comprising, but not limited to, SPR and STD-NMR.

A thermal shift assay, also called Differential Scanning Fluorimetry (DSF) is a thermal-denaturation assay that measures the thermal stability of a target protein and a subsequent increase in protein melting temperature upon binding of a ligand to the protein. The binding of low molecular weight ligands can increase the thermal stability of a protein and the thermal stability change is measured by performing a thermal denaturation curve in the presence of a fluorescent dye. The fluorescent dye used is typically a non-specific dye (such as SYPRO Orange) and binds nonspecifically to hydrophobic surfaces, and water strongly quenches the fluorescence of the fluorescent dye. When the protein unfolds, the exposed hydrophobic surfaces bind the dye, resulting in an increase in fluorescence. The stability curve and its midpoint value for the protein unfolding transition (melting temperature, Tₘ) are obtained by gradually increasing the temperature to unfold the protein and measuring the fluorescence at each point. Curves are measured for protein only and protein plus ligand, and the ΔTₘ is calculated. A positive ΔTₘ value indicates that the ligand stabilizes the protein from denaturation, and therefore binds the protein. A fluorescence-based thermal shift assay can be performed on instruments that combine sample temperature control and dye fluorescence detection, such as readily available real-time polymerase chain reaction (RT-PCR) machines.

The surface plasmon resonance (SPR) technique is a well-established method for the measurement of molecules binding to surfaces and the quantification of binding constants between surface-immobilized proteins and an analyte such as other proteins, peptides, nucleic acids, lipids or small molecules in solution without the use of labels. The SPR effect relies on changes in the refractive index of solutions adjacent to the immobilised surface and is extremely sensitive. Binding responses are measured in resonance units (RU) and are proportional to the molecular mass on the sensor chip surface and, consequently, to the number of molecules on the surface. The affinity of the interaction can be calculated from the ratio of the rate constants (K_{d}=k_{diss}/kₐₛₛ) or by a linear or nonlinear fitting of the response at equilibrium at varying concentrations of analyte.

Saturation transfer difference NMR (STD-NMR) allows for the detection of transient binding of small molecule ligands to macromolecular receptors such as proteins. In STD-NMR, magnetization transferred from the receptor to its bound ligand is measured by directly observing NMR signals from the ligand itself. Low-power irradiation is applied to a (1)H NMR spectral region containing protein signals but no ligand signals. This irradiation spreads quickly throughout the membrane protein by the process of spin diffusion and saturates all protein (1)H NMR signals. (1)H NMR signals from a ligand bound transiently to the membrane protein become saturated and, upon dissociation, serve to decrease the intensity of the (1)H NMR signals measured from the pool of free ligand. The experiment is repeated with the irradiation pulse placed outside the spectral region of protein and ligand, a condition that does not lead to saturation transfer to the ligand. The two resulting spectra are subtracted to yield the difference spectrum. The resulting difference spectrum yields only those resonances that have experienced saturation, namely those of the receptor and those of the compound that binds to the receptor. STD-NMR can therefore be used to determine the binding epitope of the compound. Competition STD-NMR methods combine STD-NMR with competition binding experiments to allow the detection of high-affinity ligands that undergo slow chemical exchange on the NMR time-scale. With this technique, the presence of a competing high-affinity ligand in the compound mixture can be detected by the disappearance or reduction of the STD signals of a low-affinity indicator ligand. This method can therefore be used to derive the binding affinity (K_{d}) of compounds based on the reduction of the signal intensity of the STD indicator.

A compound that binds to the ATP-binding site of the pseudokinase domain of the MLKL protein, as described herein, may be any compound which performs the described function and thereby effects the inhibition of necroptosis. It is emphasised that the compound is not limited to any particular chemotype, form, size, shape or conformation. Accordingly, the compound may be selected from the group consisting of synthetic compounds, organic synthetic drugs, small molecule organic drugs, natural small molecule compounds, other small molecule compounds, and peptides.

Such a compound may be identified in any manner that would be known by a person skilled in the art to be suitable. A compound may be identified through targeted drug development or through screening of commercial libraries. Such screening of commercial libraries would include medium or high throughput screening. In one embodiment, the compound is identified through screening of one or more commercial libraries.

Binding of the compound to the ATP-binding site of the pseudokinase domain of MLKL may inhibit phosphorylation of MLKL by an effector kinase or binding of the compound to the ATP-binding site of the pseudokinase domain of MLKL may not inhibit phosphorylation of MLKL by an effector kinase. The present disclosure demonstrates that compounds that bind to the ATP-binding site of the pseudokinase domain of the MLKL protein, as described herein, can inhibit necroptosis without inhibiting phosphorylation of MLKL by an effector kinase. In one embodiment, binding of the compound to the ATP-binding site of the pseudokinase domain of MLKL does not inhibit phosphorylation of MLKL by an effector kinase. In another embodiment, binding of the compound to the ATP-binding site of the pseudokinase domain of MLKL inhibits phosphorylation of MLKL by an effector kinase.

The compound as described herein may be of any suitable chemotype, form, size, shape and conformation. In one embodiment, the compound occupies a volume of up to, and including, about 1000 Å³. In one embodiment, the compound occupies a volume of up to, and including, about 900 Å³. In one embodiment, the compound occupies a volume of up to, and including, about 800 Å³. In one embodiment, the compound occupies a volume of up to, and including, about 700 Å³. In one embodiment, the compound occupies a volume of up to, and including, about 600 Å³. In another embodiment, the compound occupies a volume of from about 200 Å³ to about 900 Å³. In another embodiment, the compound occupies a volume of from about 200 Å³ to about 800 Å³. In another embodiment, the compound occupies a volume of from about 200 Å³ to about 700 Å³. In another embodiment, the compound occupies a volume of from about 300 Å³ to about 900 Å³. In another embodiment, the compound occupies a volume of from about 300 Å³ to about 800 Å³. In another embodiment, the compound occupies a volume of from about 300 Å³ to about 700 Å³. In another embodiment, the compound occupies a volume of from about 400 Å³ to about 600 Å³. In another embodiment, the compound occupies a volume of from about 300 Å³ to about 600 Å³. In another embodiment, the compound occupies a volume of from about 200 Å³ to about 600 Å³. In another embodiment, the compound occupies a volume of from about 200 Å³ to about 500 Å³. In yet another embodiment, the compound occupies a volume of from about 200 Å³ to about 400 Å³.

In one embodiment of the present disclosure, the compound has the formula

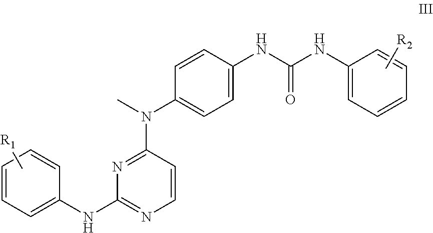

wherein: R₁ is selected from the group consisting of 3-MeSO₂CH₂—, 4-MeSO₂CH₂—, 3-H₂NSO₂— and 4-H₂NSO₂—; and
R₂ is 0-2 substituents independently selected from the group selected from the group consisting of OCF₃, CF₃, fluoro, chloro, bromo, iodo and COMe,

or a pharmaceutically acceptable derivative, polymorph, salt or prodrug thereof. In one embodiment, R₁ is 3-H₂NSO₂—. In another embodiment, R₁ is 4-H₂NSO₂—.

In one embodiment, R₂ is 0 substituents. In another embodiment, R₂ is 4-OCF₃. In yet another embodiment, R₂ is 2 substituents, and wherein the 2 substituents are 3-CF₃ and 6-fluoro.

In a further embodiment, the compound of Formula III is selected from the group consisting of compounds 1 to 4

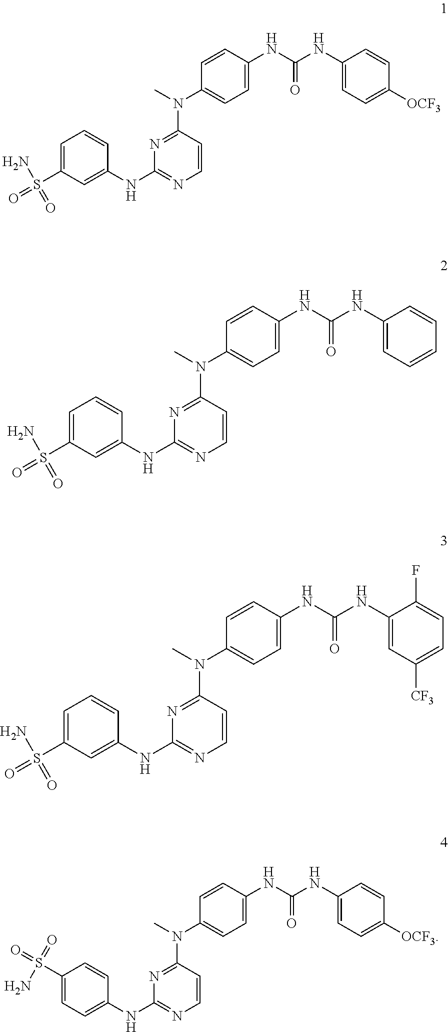 [SMILES: CN(c1ccc(NC(=O)Nc2ccc(OC(F)(F)F)cc2)cc1)c1ccnc(Nc2cccc(S(N)(=O)=O)c2)n1 ; CN(c1ccc(NC(=O)Nc2ccccc2)cc1)c1ccnc(Nc2cccc(S(N)(=O)=O)c2)n1 ; CN(c1ccc(NC(=O)Nc2cc(C(F)(F)F)ccc2F)cc1)c1ccnc(Nc2cccc(S(N)(=O)=O)c2)n1 ; CN(c1ccc(NC(=O)Nc2ccc(OC(F)(F)F)cc2)cc1)c1ccnc(Nc2ccc(S(N)(=O)=O)cc2)n1]

In yet another embodiment, the compound of Formula III is compound 1

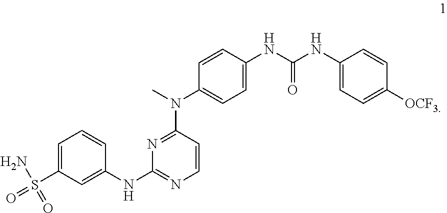 [SMILES: CN(c1ccc(NC(=O)Nc2ccc(OC(F)(F)F)cc2)cc1)c1ccnc(Nc2cccc(S(N)(=O)=O)c2)n1]

In one aspect, the compound for inhibiting necroptosis has a formula according to Formula (I):

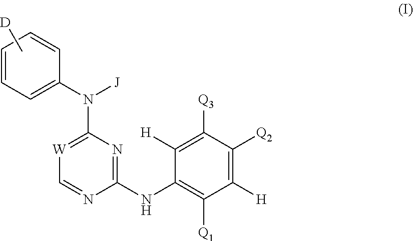

or a salt, solvate, or physiologically functional derivative thereof:

wherein:
W is N or C—R, wherein R is hydrogen, halogen, or cyano;
J is hydrogen, C₁-C₄ alkyl, C₁-C₄ haloalkyl, aralkyl, cyanoalkyl, —(CH₂)ₚC═CH(CH₂)ₜH, —(CH₂)ₚC≡C(CH₂)ₜH, or C₃-C₇ cycloalkyl;
p is 1, 2, or 3;
t is 0 or 1;
D is —N(H)(X);
X is the group defined by —(X₁)—(X₂)_{q}—(X₃) wherein
X₁ is C(O) or C(S) and q is 1, or
X₁ is —C(O) or —S(O)₂ and q is 0,
X₂ is N(H) or O, and
X₃ is alkyl, cycloalkyl, heterocyclyl, alkoxy, aryloxy, aralkoxy, aryl, aralkyl, or heteroaryl, or
alkyl, cycloalkyl, heterocyclyl, alkoxy, aryloxy, aralkoxy, aryl, aralkyl, or heteroaryl substituted with at least one group defined by —(X₄)_{z}—(X₅),
   X₄ is C(H)₂ where z is 0, 1, 2, 3, or 4, and
   X₅ is hydrogen, C₁-C₆ alkyl, C₁-C₆ haloalkyl, cycloalkyl, heterocyclyl, aryl, heteroaryl, alkoxy, haloalkoxy, hydroxy, aryloxy, aralkoxy, halo, —CN, —NR′R′, N(H)C(O)R″, N(H)C(O)OR″, N(H)C(O)NR′R′, N(H)S(O)₂R″, OR″, OC(O)RR″, C(O)R″, SR″, —S(O)R′″, S(O)₂R′″,— or S(O)₂NR′R′, where,
   R′ is hydrogen, alkyl, cycloalkyl, heterocyclyl, —OR¹, —SR¹, —S(O)₂R¹, —S(O)R¹, or C(O)R¹;
   R″ is hydrogen, alkyl, cycloalkyl, heterocyclyl, —OR¹, —NR³R⁴, —S(O)₂R¹, —S(O)R¹ or C(O)R¹; and
   R′″ is hydrogen, alkyl, cycloalkyl, heterocyclyl, —OR¹ or —NR³R⁴;
   Q₁ is hydrogen, halogen, C₁-C₂ haloalkyl, C₁-C₂ alkyl, C₁-C₂ alkoxy, or C₁-C₂ haloalkoxy;
   Q₂ is A¹ or A²;
   Q₃ is A¹ when Q₂ is A² and Q₃ is A² when Q₂ is A¹;
   wherein
   A¹ is hydrogen, halogen, C₁-C₃ alkyl, C₁-C₃ haloalkyl, —OR¹, and
A² is the group defined by —(Z)ₘ—(Z¹)—(Z²), wherein
   Z is CH₂ and m is 0, 1, 2, or 3, or
   Z is NR² and m is 0 or 1, or
   Z is oxygen and m is 0 or 1, or
   Z is CH₂NR² and m is 0 or 1;
   Z¹ is S(O)₂, S(O), or C(O); and
   Z² is C₁-C₄ alkyl, cycloalkyl, heterocyclyl, NR³R⁴, aryl, arylamino, aralkyl, aralkoxy, or heteroaryl;
R¹ is hydrogen, alkyl, heterocyclyl, and —NR³R⁴;
R², R³, and R⁴ are each independently selected from hydrogen, hydroxy, alkoxy, aryloxy, aralkoxy, amino, alkylamino, arylamino, aralkylamino, C₁-C₄ alkyl, C₃-C₇ cycloalkyl, heterocyclyl, —S(O)₂R⁵, and —C(O)R⁵; and
R⁵ is C₁-C₄ alkyl, or C₃-C₇ cycloalkyl.

In another aspect, the compound for inhibiting necroptosis has a formula according to Formula (II):

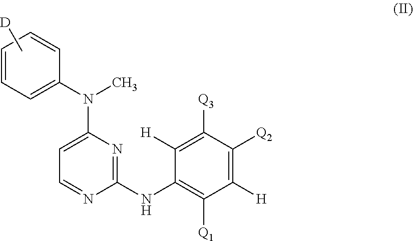

or a salt, solvate, or physiologically functional derivative thereof:
wherein:
   D is —N(H)(X);
   X is the group defined by —(X₁)—(X₂)_{q}—(X₃) wherein
   X₁ is C(O) or C(S) and q is 1, or
   X₁ is —C(O) or —S(O)₂ and q is 0,
   X₂ is N(H) or O, and
   X₃ is alkyl, cycloalkyl, heterocyclyl, alkoxy, aryloxy, aralkoxy, aryl, aralkyl, or heteroaryl, or
   alkyl, cycloalkyl, heterocyclyl, alkoxy, aryloxy, aralkoxy, aryl, aralkyl, or heteroaryl substituted with at least one group defined by —(X₄)_{z}—(X₅),
   X₄ is C(H)₂ where z is 0, 1, 2, 3, or 4, and
   X₅ is hydrogen, C₁-C₆ alkyl, C₁-C₆ haloalkyl, cycloalkyl, heterocyclyl, aryl, heteroaryl, alkoxy, haloalkoxy, hydroxy, aryloxy, aralkoxy, halo, —CN, —NR′R′, N(H)C(O)R″, N(H)C(O)OR″, N(H)C(O)NR′R′, N(H)S(O)₂R″, OR″, OC(O)R″, C(O)R″, SR″, —S(O)R′″, S(O)₂R′″, -or S(O)₂NR′R′, where,
   R′ is hydrogen, alkyl, cycloalkyl, heterocyclyl, —OR¹, —SR¹, —S(O)₂R¹, —S(O)R¹, or C(O)R¹;
   R″ is hydrogen, alkyl, cycloalkyl, heterocyclyl, —OR¹, —NR³R⁴, —S(O)₂R¹, —S(O)R¹, or C(O)R¹; and
   R′″ is hydrogen, alkyl, cycloalkyl, heterocyclyl, —OR¹, or —NR³R⁴;
   Q₁ is hydrogen, halogen, C₁-C₂ haloalkyl, C₁-C₂alkyl, C₁-C₂alkoxy, or C₁-C₂ haloalkoxy;
   Q₂ is A¹ or A²;
   Q₃ is A¹ when Q₂ is A² and Q₃ is A² when Q₂ is A¹;
   wherein
      A¹ is hydrogen, halogen, C₁-C₃ alkyl, C₁-C₃ haloalkyl, —OR, and
      A² is the group defined by —(Z)ₘ—(Z¹)—(Z²), wherein
      Z is CH₂ and m is 0, 1, 2, or 3, or
      Z is NR² and m is 0 or 1, or
      Z is oxygen and m is 0 or 1, or
      Z is CH₂NR² and m is 0 or 1;
      Z¹ is S(O)₂, S(O), or C(O); and
      Z² is C₁-C₄ alkyl, cycloalkyl, heterocyclyl, NR³R⁴, aryl, arylamino, aralkyl, aralkoxy, or heteroaryl;
   R¹ is hydrogen, heterocyclyl, and —NR³R⁴;
   R², R³, and R⁴ are each independently selected from hydrogen, hydroxy, alkoxy, aryloxy, aralkoxy, amino, alkylamino, arylamino, aralkylamino, C₁-C₄ alkyl, C₃-C₇ cycloalkyl, heterocyclyl, —S(O)₂R⁵, and —C(O)R⁵; and
   R⁵ is C₁-C₄ alkyl, or C₃-C₇ cycloalkyl.

In another aspect, the compound for inhibiting necroptosis has a formula according to Formula (I):

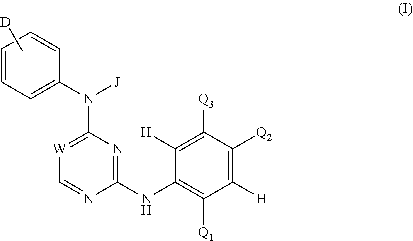

or a salt, solvate, or physiologically functional derivative thereof:

wherein:
W is N or C—R, wherein R is hydrogen, halogen, or cyano;
J is hydrogen, C₁-C₄alkyl, C₁-C₄ haloalkyl, aralkyl, cyanoalkyl, —(CH₂)ₚC═CH(CH₂)ₜH, —(CH₂)ₚC≡C(CH₂)ₜH, or C₃-C₇ cycloalkyl;
   p is 1, 2, or 3;
   t is 0 or 1;
   D is

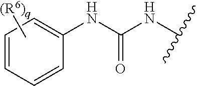

q is 1, 2, or 3;
Q₁ is hydrogen, halogen, C₁-C₂ haloalkyl, C₁-C₂ alkyl, C₁-C₂ alkoxy, or C₁-C₂ haloalkoxy;
Q₂ is A¹ or A²;
Q₃ is A¹ when Q₂ is A² and Q₃ is A² when Q₂ is A¹;
wherein
   A¹ is hydrogen, halogen, C₁-C₃ alkyl, C₁-C₃ haloalkyl, —OR¹′ and
   A² is the group defined by —(Z)ₘ—(Z¹)—(Z²),
wherein
   Z is CH₂ and m is 0, 1, 2, or 3, or
   Z is NR² and m is 0 or 1, or
   Z is O and m is 0 or 1, or
   Z is CH₂NR² and m is 0 or 1;
   Z¹ is S(O)₂, S(O), or C(O); and
   Z² is C₁-C₄ alkyl, cycloalkyl, heterocyclyl, NR³R⁴, aryl, arylamino, aralkyl, aralkoxy, or heteroaryl;
      R², R³, and R⁴ are each independently selected from hydrogen, hydroxy, alkoxy, aryloxy, aralkoxy, amino, alkylamino, arylamino, aralkylamino, C₁-C₄ alkyl, C₃-C₇ cycloalkyl, heterocyclyl, —S(O)₂R⁵, and —C(O)R⁵;
      R⁵ is C₁-C₆alkyl, or C₃-C₇ cycloalkyl; and
      R⁶ is the group defined by —(X₄)_{z}—(X₅),
   wherein
      X₄ is C(H)₂ where z is 0, 1, 2, 3, or 4, and
      X₅ is hydrogen, C₁-C₆ alkyl, C₁-C₆ haloalkyl, cycloalkyl, heterocyclyl, aryl, heteroaryl, alkoxy, haloalkoxy, hydroxy, aryloxy, aralkoxy, halo, CN, —NR⁷R⁷, —N(H)C(O)R⁷, —N(H)C(O)ORR⁷R⁷N(H)S(O)₂R⁷, N(H)S(O)₂NR⁷R⁷, —OC(O)R⁷, OC(O)NR⁷R⁷, —C(O)R⁷, —C(O)NR⁷R⁷, —SR⁷, —S(O)R⁷, S(O)₂R⁷R⁷, or —S(O)₂NR⁷R⁷; and
   R⁷ is hydrogen, C₁-C₆ alkyl, C₁-C₆ haloalkyl, cycloalkyl, heterocyclyl, alkylamino, alkoxy, aryloxy, aralkoxy, arylamino, aralkylamino, aryl or heteroaryl.

In yet another aspect, the compound for inhibiting necroptosis has a formula according to Formula (II):

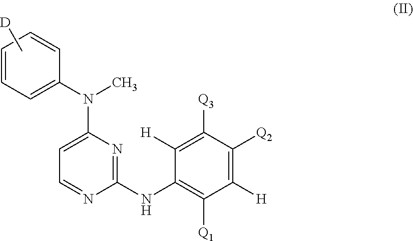

or a salt, solvate, or physiologically functional derivative thereof:

wherein:
D is

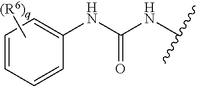

q is 1, 2, or 3;
Q₁ is hydrogen, halogen, C₁-C₂ haloalkyl, C₁-C₂ alkyl, C₁-C₂ alkoxy, or C₁-C₂ haloalkoxy;
Q₂ is A¹ or A²;
Q₃ is A when Q₂ is A² and Q₃ is A² when Q₂ is A¹; wherein
   A¹ is hydrogen, halogen, C₁-C₃alkyl, C₁-C₃ haloalkyl, —OR¹, and
   A² is the group defined by —(Z)ₘ—(Z¹)—(Z²),
wherein
   Z is CH₂ and m is 0, 1, 2, or 3, or
   Z is NR² and m is 0 or 1, or
   Z is O and m is 0 or 1, or
   Z is CH₂NR² and m is 0 or 1;
   Z¹ is S(O)₂, S(O), or C(O); and
   Z² is C₁-C₄ alkyl, cycloalkyl, heterocyclyl, NR³R⁴, aryl, arylamino, aralkyl, aralkoxy, or heteroaryl;
      R², R³, and R⁴ are each independently selected from hydrogen, hydroxy, alkoxy, aryloxy, aralkoxy, amino, alkylamino, arylamino, aralkylamino, C₁-C₄ alkyl, C₃-C₇ cycloalkyl, heterocyclyl, —S(O)₂RS, and —C(O)R⁵;
      R⁵ is C₁-C₆alkyl, or C₃-C₇ cycloalkyl; and
      R⁶ is the group defined by —(X₄)_{z}—(X₅), wherein
         X₄ is C(H)₂ where z is 0, 1, 2, 3, or 4, and
         X₅ is hydrogen, C₁-C₆ alkyl, C₁-C₆ haloalkyl, cycloalkyl, heterocyclyl, aryl, heteroaryl, alkoxy, haloalkoxy, hydroxy, aryloxy, aralkoxy, halo, CN, —NR⁷R⁷, —N(H)C(O)R⁷, —N(H)C(O)OR⁷, —N(H)C(O)NR⁷R⁷, N(H)S(O)₂R⁷, N(H)S(O)₂NR⁷R⁷, —OC(O)R⁷, OC(O)NR⁷R⁷, —C(O)R⁷, —O)NR⁷R⁷, —SR⁷, —S(O)R⁷, —S(O)₂R⁷R⁷, or —S(O)₂NR⁷R⁷; and
         R⁷ is hydrogen, C₁-C₆ alkyl, C₁-C₆ haloalkyl, cycloalkyl, heterocyclyl, alkylamino, alkoxy, aryloxy, aralkoxy, arylamino, aralkylamino, aryl or heteroaryl.

In another aspect, the compound for inhibiting necroptosis has a formula according to Formula (I):

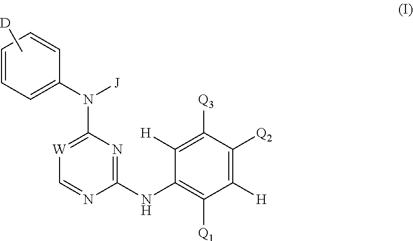

or a salt, solvate, or physiologically functional derivative thereof:
wherein:
   W is N or C—R, wherein R is hydrogen, halogen, or cyano;
   J is hydrogen, C₁-C₄alkyl, C₁-C₄ haloalkyl, aralkyl, cyanoalkyl, —(CH₂)ₚC═CH(CH₂)ₜH, —(CH₂)ₚC═C(CH₂)ₜH, or C₃-C₇ cycloalkyl;
   p is 1, 2, or 3;
   t is 0 or 1;
   D is —N(R⁸)(X);
   X is the group defined by —(X₁)—(X₂)_{q}—(X₃)
wherein
   X₁ is C(O) or C(S) and q is 1, or
   X₁ is —C(O) or —S(O)₂ and q is 0,
   X₂ is N(H) or O, and
   X₃ is alkyl, cycloalkyl, heterocyclyl, alkoxy, aryloxy, aralkoxy, aryl, aralkyl, or heteroaryl, or
   alkyl, cycloalkyl, heterocyclyl, alkoxy, aryloxy, aralkoxy, aryl, aralkyl, or heteroaryl substituted with at least one group defined by —(X₄)_{z}—(X₅),
   X₄ is C(H)₂ where z is 0, 1, 2, 3, or 4, and
   X₅ is hydrogen, C₁-C₆ alkyl, C₁-C₆ haloalkyl, cycloalkyl, heterocyclyl, aryl, heteroaryl, alkoxy, haloalkoxy, hydroxy, aryloxy, aralkoxy, halo, —CN, —NR′R′, N(H)C(O)R″, N(H)C(O)OR″, N(H)C(O)NR′R′, N(H)S(O)₂R″, OR″, OC(O)R″, C(O)R″, SR″, —S(O)R′″, —S(O)₂R′″, or —S(O)₂NR′R′, where,
      R′ is hydrogen, alkyl, cycloalkyl, heterocyclyl, —OR¹, —SR¹, —S(O)₂R¹, —S(O)R¹, or C(O)R¹;
      R″ is hydrogen, alkyl, cycloalkyl, heterocyclyl, —OR¹, —NR³R⁴, —S(O)₂R¹, —S(O)R¹, or C(O)R¹; and
      R′″ is hydrogen, alkyl, cycloalkyl, heterocyclyl, —OR¹, or —NR³R⁴;
   Q₁ is hydrogen, halogen, C₁-C₂ haloalkyl, C₁-C₂ alkyl, C₁-C₂ alkoxy, or C₁-C₂ haloalkoxy;
   Q₂ is A¹ or A²;
   Q₃ is A¹ when Q₂ is A² and Q₃ is A² when Q₂ is A¹;
   wherein
      A¹ is hydrogen, halogen, C₁-C₃alkyl, C₁-C₃ haloalkyl, —OR¹, and
      A² is the group defined by —(Z)ₘ—(Z¹)—(Z²),
   wherein
      Z is CH₂ and m is 0, 1, 2, or 3, or
      Z is NR² and m is 0 or 1, or
      Z is oxygen and m is 0 or 1, or
      Z is CH₂NR² and m is 0 or 1;
      Z¹ is S(O)₂, S(O), or C(O); and
      Z² is C₁-C₄ alkyl, cycloalkyl, heterocyclyl, NR³R⁴, aryl, arylamino, aralkyl, aralkoxy, or heteroaryl;
   R¹ is hydrogen, alkyl, heterocyclyl, and —NR³R⁴;
   R², R³, and R⁴ are each independently selected from hydrogen, hydroxy, alkoxy, aryloxy, aralkoxy, amino, alkylamino, arylamino, aralkylamino, C₁-C₄ alkyl, C₃-C₇ cycloalkyl, heterocyclyl, —S(O)₂R⁵, and —C(O)R⁵;
      R⁵ is C₁-C₄ alkyl, or C₃-C₇ cycloalkyl; and
      R⁸ is hydrogen or C₁-C₃ alkyl.

In another aspect, the invention provides a method for inhibiting necroptosis in a subject in need thereof, the method comprising administering a therapeutically effective amount of a compound according to Formula (I):

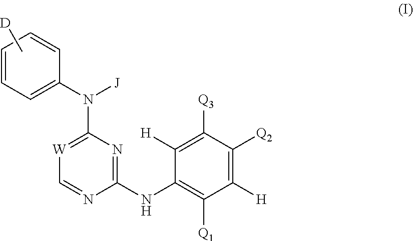

or a salt, solvate, or physiologically functional derivative thereof,

wherein:
W is N or C—R, wherein R is hydrogen, halogen, or cyano;
J is hydrogen, C₁-C₄ alkyl, C₁-C₄ haloalkyl, aralkyl, cyanoalkyl, —(CH₂)ₚC═CH(CH₂)ₜH, —(CH₂)ₚC≡C(CH₂)ₜH, or C₃-C₇ cycloalkyl;
p is 1, 2, or 3;
t is 0 or 1;
D is —N(H)(X);
X is the group defined by —(X₁)—(X₂)_{q}—(X₃) wherein
X₁ is C(O) or C(S) and q is 1, or
X₁ is —C(O) or —S(O)₂ and q is 0,
X₂ is N(H) or O, and
X₃ is alkyl, cycloalkyl, heterocyclyl, alkoxy, aryloxy, aralkoxy, aryl, aralkyl, or heteroaryl, or
alkyl, cycloalkyl, heterocyclyl, alkoxy, aryloxy, aralkoxy, aryl, aralkyl, or heteroaryl substituted with at least one group defined by —(X₄)_{z}—(X₅),
   X₄ is C(H)₂ where z is 0, 1, 2, 3, or 4, and
   X₅ is hydrogen, C₁-C₆ alkyl, C₁-C₆ haloalkyl, cycloalkyl, heterocyclyl, aryl, heteroaryl, alkoxy, haloalkoxy, hydroxy, aryloxy, aralkoxy, halo, —CN, —NR′R′, N(H)C(O)R″, N(H)C(O)OR″, N(H)C(O)NR′R′, N(H)S(O)₂R″, OR″, OC(O)RR″, C(O)R″, SR″, —S(O)R′″, S(O)₂R′″,— or S(O)₂NR′R′, where,
   R′ is hydrogen, alkyl, cycloalkyl, heterocyclyl, —OR¹, —SR¹, —S(O)₂R¹, —S(O)R¹, or C(O)R;
   R″ is hydrogen, alkyl, cycloalkyl, heterocyclyl, —OR¹, —NR³R⁴, —S(O)₂R¹, —S(O)R¹ or C(O)R¹; and
   R′″ is hydrogen, alkyl, cycloalkyl, heterocyclyl, —OR¹ or —NR³R⁴;
   Q₁ is hydrogen, halogen, C₁-C₂ haloalkyl, C₁-C₂ alkyl, C₁-C₂ alkoxy, or C₁-C₂ haloalkoxy;
   Q₂ is A¹ or A²;
   Q₃ is A¹ when Q₂ is A² and Q₃ is A² when Q₂ is A¹;
   wherein
   A¹ is hydrogen, halogen, C₁-C₃ alkyl, C₁-C₃ haloalkyl, —OR¹, and
   A² is the group defined by —(Z)ₘ—(Z¹)—(Z²), wherein
      Z is CH₂ and m is 0, 1, 2, or 3, or
      Z is NR² and m is 0 or 1, or
      Z is oxygen and m is 0 or 1, or
      Z is CH₂NR² and m is 0 or 1;
      Z¹ is S(O)₂, S(O), or C(O); and
      Z² is C₁-C₄ alkyl, cycloalkyl, heterocyclyl, NR³R⁴, aryl, arylamino, aralkyl, aralkoxy, or heteroaryl;
   R¹ is hydrogen, alkyl, heterocyclyl, and —NR³R⁴;
   R², R³, and R⁴ are each independently selected from hydrogen, hydroxy, alkoxy, aryloxy, aralkoxy, amino, alkylamino, arylamino, aralkylamino, C₁-C₄ alkyl, C₃-C₇ cycloalkyl, heterocyclyl, —S(O)₂R⁵, and —C(O)R⁵; and
      R⁵ is C₁-C₄ alkyl, or C₃-C₇ cycloalkyl.

In another aspect, the invention provides a method for inhibiting necroptosis in a subject in need thereof, the method comprising administering a therapeutically effective amount of a compound according to Formula (II):

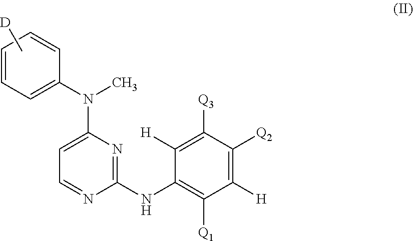

or a salt, solvate, or physiologically functional derivative thereof:
wherein:
   D is —N(H)(X);
   X is the group defined by —(X₁)—(X₂)_{q}—(X₃) wherein
   X₁ is C(O) or C(S) and q is 1, or
   X₁ is —C(O) or —S(O)₂ and q is 0,
   X₂ is N(H) or O, and
   X₃ is alkyl, cycloalkyl, heterocyclyl, alkoxy, aryloxy, aralkoxy, aryl, aralkyl, or heteroaryl, or
   alkyl, cycloalkyl, heterocyclyl, alkoxy, aryloxy, aralkoxy, aryl, aralkyl, or heteroaryl substituted with at least one group defined by —(X₄)_{z}—(X₅),
   X₄ is C(H)₂ where z is 0, 1, 2, 3, or 4, and
   X₅ is hydrogen, C₁-C₆ alkyl, C₁-C₆ haloalkyl, cycloalkyl, heterocyclyl, aryl, heteroaryl, alkoxy, haloalkoxy, hydroxy, aryloxy, aralkoxy, halo, —CN, —NR′R′, N(H)C(O)R″, N(H)C(O)OR″, N(H)C(O)NR′R′, N(H)S(O)₂R″, OR″, OC(O)R″, C(O)R″, SR″, —S(O)R′″, —S(O)₂R′″, or —S(O)₂NR′R′, where,
   R′ is hydrogen, alkyl, cycloalkyl, heterocyclyl, —OR¹, —SR¹, —S(O)₂R¹, —S(O)R¹, or C(O)R¹;
   R″ is hydrogen, alkyl, cycloalkyl, heterocyclyl, —OR¹, —NR³R⁴, —S(O)₂R¹, —S(O)R¹, or C(O)R¹; and
   R′″ is hydrogen, alkyl, cycloalkyl, heterocyclyl, —OR¹, or —NR³R⁴; Q₁ is hydrogen, halogen, C₁-C₂ haloalkyl, C₁-C₂alkyl, C₁-C₂alkoxy, or C₁-C₂ haloalkoxy;
   Q₂ is A¹ or A²;
   Q₃ is A¹ when Q₂ is A² and Q₃ is A² when Q₂ is A¹;
   wherein
      A¹ is hydrogen, halogen, C₁-C₃ alkyl, C₁-C₃ haloalkyl, —OR¹, and
      A² is the group defined by —(Z)ₘ—(Z¹)—(Z²),
   wherein
      Z is CH₂ and m is 0, 1, 2, or 3, or
      Z is NR² and m is 0 or 1, or
      Z is oxygen and m is 0 or 1, or
      Z is CH₂NR² and m is 0 or 1;
      Z¹ is S(O)₂, S(O), or C(O); and
      Z² is C₁-C₄ alkyl, cycloalkyl, heterocyclyl, NR³R⁴, aryl, arylamino, aralkyl, aralkoxy, or heteroaryl;
   R¹ is hydrogen, heterocyclyl, and —NR³R⁴;
   R², R³, and R⁴ are each independently selected from hydrogen, hydroxy, alkoxy, aryloxy, aralkoxy, amino, alkylamino, arylamino, aralkylamino, C₁-C₄ alkyl, C₃-C₇ cycloalkyl, heterocyclyl, —S(O)₂R⁵, and —C(O)R⁵; and
R⁵ is C₁-C₄ alkyl, or C₃-C₇ cycloalkyl.

In another aspect, the invention provides a method for inhibiting necroptosis in a subject in need thereof, the method comprising administering a therapeutically effective amount of a compound according to Formula (I):

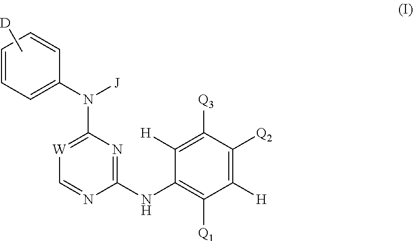

or a salt, solvate, or physiologically functional derivative thereof:

wherein:
W is N or C—R, wherein R is hydrogen, halogen, or cyano;
J is hydrogen, C₁-C₄alkyl, C₁-C₄ haloalkyl, aralkyl, cyanoalkyl, —(CH₂)ₚC═CH(CH₂)ₜH, —(CH₂)ₚC≡C(CH₂)ₜH, or C₃-C₇ cycloalkyl;
p is 1, 2, or 3;
t is 0 or 1;
D is

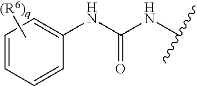

q is 1, 2, or 3;
Q₁ is hydrogen, halogen, C₁-C₂ haloalkyl, C₁-C₂ alkyl, C₁-C₂ alkoxy, or C₁-C₂ haloalkoxy;
Q₂ is A¹ or A²;
Q₃ is A¹ when Q₂ is A² and Q₃ is A² when Q₂ is A¹;
wherein
   A¹ is hydrogen, halogen, C₁-C₃ alkyl, C₁-C₃ haloalkyl, —OR¹′ and
   A² is the group defined by —(Z)ₘ—(Z¹)—(Z²),
wherein
   Z is CH₂ and m is 0, 1, 2, or 3, or
   Z is NR² and m is 0 or 1, or
   Z is O and m is 0 or 1, or
   Z is CH₂NR² and m is 0 or 1;
   Z¹ is S(O)₂, S(O), or C(O); and
   Z² is C₁-C₄ alkyl, cycloalkyl, heterocyclyl, NR³R⁴, aryl, arylamino, aralkyl, aralkoxy, or heteroaryl;
   R², R³, and R⁴ are each independently selected from hydrogen, hydroxy, alkoxy, aryloxy, aralkoxy, amino, alkylamino, arylamino, aralkylamino, C₁-C₄ alkyl, C₃-C₇ cycloalkyl, heterocyclyl, —S(O)₂R⁵, and —C(O)R⁵;
   R⁵ is C₁-C₆alkyl, or C₃-C₇ cycloalkyl; and
   R⁶ is the group defined by —(X₄)_{z}—(X₅),
wherein
   X₄ is C(H)₂ where z is 0, 1, 2, 3, or 4, and
   X₅ is hydrogen, C₁-C₆ alkyl, C₁-C₆ haloalkyl, cycloalkyl, heterocyclyl, aryl, heteroaryl, alkoxy, haloalkoxy, hydroxy, aryloxy, aralkoxy, halo, CN, —NR⁷R⁷, —N(H)C(O)R⁷, —N(H)C(O)ORR⁷R⁷N(H)S(O)₂R⁷, N(H)S(O)₂NR⁷R⁷, —OC(O)R⁷, OC(O)NR⁷R⁷, —C(O)R⁷, —C(O)NR⁷R⁷, —SR⁷, —S(O)R⁷, —S(O)₂R⁷R⁷, or —S(O)₂NR⁷R⁷; and
   R⁷ is hydrogen, C₁-C₆ alkyl, C₁-C₆ haloalkyl, cycloalkyl, heterocyclyl, alkylamino.

In another aspect, the invention provides a method for inhibiting necroptosis in a subject in need thereof, the method comprising administering a therapeutically effective amount of a compound according to Formula (II):

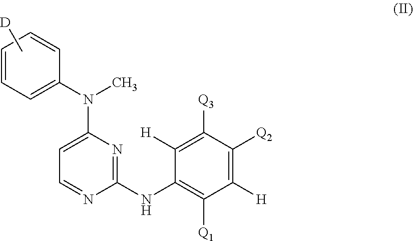

or a salt, solvate, or physiologically functional derivative thereof:

wherein:
D is

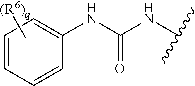

q is 1, 2, or 3;
Q₁ is hydrogen, halogen, C₁-C₂ haloalkyl, C₁-C₂ alkyl, C₁-C₂ alkoxy, or C₁-C₂ haloalkoxy;
Q₂ is A¹ or A²;
Q₃ is A when Q₂ is A² and Q₃ is A² when Q₂ is A¹; wherein
   A¹ is hydrogen, halogen, C₁-C₃alkyl, C₁-C₃ haloalkyl, —OR¹, and
   A² is the group defined by —(Z)ₘ—(Z¹)—(Z²),
wherein
   Z is CH₂ and m is 0, 1, 2, or 3, or
   Z is NR² and m is 0 or 1, or
   Z is O and m is 0 or 1, or
   Z is CH₂NR² and m is 0 or 1;
   Z¹ is S(O)₂, S(O), or C(O); and
   Z² is C₁-C₄ alkyl, cycloalkyl, heterocyclyl, NR³R⁴, aryl, arylamino, aralkyl, aralkoxy, or heteroaryl;
   R², R³, and R⁴ are each independently selected from hydrogen, hydroxy, alkoxy, aryloxy, aralkoxy, amino, alkylamino, arylamino, aralkylamino, C₁-C₄ alkyl, C₃-C₇ cycloalkyl, heterocyclyl, —S(O)₂RS, and —C(O)R⁵;
   R⁵ is C₁-C₆alkyl, or C₃-C₇ cycloalkyl; and
   R⁶ is the group defined by —(X₄)_{z}—(X₅), wherein
      X₄ is C(H)₂ where z is 0, 1, 2, 3, or 4, and
      X₅ is hydrogen, C₁-C₆ alkyl, C₁-C₆ haloalkyl, cycloalkyl, heterocyclyl, aryl, heteroaryl, alkoxy, haloalkoxy, hydroxy, aryloxy, aralkoxy, halo, CN, —NR⁷R⁷, —N(H)C(O)R⁷, —N(H)C(O)OR⁷, —N(H)C(O)NR⁷R⁷, N(H)S(O)₂R⁷, N(H)S(O)₂NR⁷R⁷, —OC(O)R⁷, OC(O)NR⁷R⁷, —C(O)R⁷, —O)NR⁷R⁷, —SR⁷, —S(O)R⁷, —S(O)₂R⁷R⁷, or —S(O)₂NR⁷R⁷; and
         R⁷ is hydrogen, C₁-C₆ alkyl, C₁-C₆ haloalkyl, cycloalkyl, heterocyclyl, alkylamino, alkoxy, aryloxy, aralkoxy, arylamino, aralkylamino, aryl or heteroaryl.

In another aspect, the invention provides a method for inhibiting necroptosis in a subject in need thereof, the method comprising administering a therapeutically effective amount of a compound according to Formula (I):

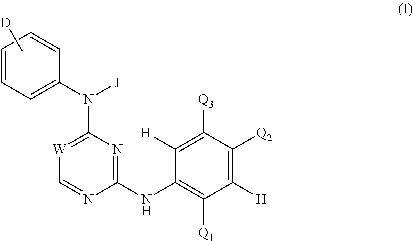

or a salt, solvate, or physiologically functional derivative thereof:

wherein:
W is N or C—R, wherein R is hydrogen, halogen, or cyano;
J is hydrogen, C₁-C₄alkyl, C₁-C₄ haloalkyl, aralkyl, cyanoalkyl, —(CH₂)ₚC═CH(CH₂)ₜH, —(CH₂)ₚC═C(CH₂)ₜH, or C₃-C₇ cycloalkyl;
p is 1, 2, or 3;
t is 0 or 1;
D is —N(R⁸)(X);
X is the group defined by —(X₁)—(X₂)_{q}—(X₃)

wherein
X₁ is C(O) or C(S) and q is 1, or
X₁ is —C(O) or —S(O)₂ and q is 0,
X₂ is N(H) or O, and
X₃ is alkyl, cycloalkyl, heterocyclyl, alkoxy, aryloxy, aralkoxy, aryl, aralkyl, or heteroaryl, or
alkyl, cycloalkyl, heterocyclyl, alkoxy, aryloxy, aralkoxy, aryl, aralkyl, or heteroaryl substituted with at least one group defined by —(X₄)_{z}—(X₅),
X₄ is C(H)₂ where z is 0, 1, 2, 3, or 4, and
X₅ is hydrogen, C₁-C₆ alkyl, C₁-C₆ haloalkyl, cycloalkyl, heterocyclyl, aryl, heteroaryl, alkoxy, haloalkoxy, hydroxy, aryloxy, aralkoxy, halo, —CN, —NR′R′, N(H)C(O)R″, N(H)C(O)OR″, N(H)C(O)NR′R′, N(H)S(O)₂R″, OR″, OC(O)R″, C(O)R″, SR″, —S(O)R′″, —S(O)₂R′″, or —S(O)₂NR′R′, where,
   R′ is hydrogen, alkyl, cycloalkyl, heterocyclyl, —OR¹, —SR¹, —S(O)₂R¹, —S(O)R¹, or C(O)R¹;
   R″ is hydrogen, alkyl, cycloalkyl, heterocyclyl, —OR¹, —NR³R⁴, —S(O)₂R¹, —S(O)R¹, or C(O)R¹; and
   R′″ is hydrogen, alkyl, cycloalkyl, heterocyclyl, —OR¹, or —NR³R⁴;
Q₁ is hydrogen, halogen, C₁-C₂ haloalkyl, C₁-C₂ alkyl, C₁-C₂ alkoxy, or C₁-C₂ haloalkoxy;
Q₂ is A¹ or A²;
Q₃ is A¹ when Q₂ is A² and Q₃ is A² when Q₂ is A¹;
wherein
   A¹ is hydrogen, halogen, C₁-C₃alkyl, C₁-C₃ haloalkyl, —OR¹, and
   A² is the group defined by —(Z)ₘ—(Z¹)—(Z²),
wherein
   Z is CH₂ and m is 0, 1, 2, or 3, or
   Z is NR² and m is 0 or 1, or
   Z is oxygen and m is 0 or 1, or
   Z is CH₂NR² and m is 0 or 1;
   Z¹ is S(O)₂, S(O), or C(O); and
   Z² is C₁-C₄ alkyl, cycloalkyl, heterocyclyl, NR³R⁴, aryl, arylamino, aralkyl, aralkoxy, or heteroaryl;
R¹ is hydrogen, alkyl, heterocyclyl, and —NR³R⁴;
R², R³, and R⁴ are each independently selected from hydrogen, hydroxy, alkoxy, aryloxy, aralkoxy, amino, alkylamino, arylamino, aralkylamino, C₁-C₄ alkyl, C₃-C₇ cycloalkyl, heterocyclyl, —S(O)₂R⁵, and —C(O)R⁵;
R⁵ is C₁-C₄ alkyl, or C₃-C₇ cycloalkyl; and
R⁸ is hydrogen or C₁-C₃ alkyl.

In another aspect, there is further provided novel compounds of formulas (I), (II) and/or (III).

In another aspect, there is provided use of a compound of Formula (I), (II) and/or (III) in the preparation of a medicament for the inhibition of necroptosis in a subject.

In another aspect, there is provided use of a compound according to Formula (I), (II) and/or (III) for inhibiting necroptosis.

In yet another aspect, there is provided a compound according to Formula (I), (II) and/or (III) for use in inhibiting necroptosis.

In yet another aspect, there is provided a compound according to Formula (I), (II) and/or (III) when used for inhibiting necroptosis.

The salts of the compound of formulas I, II and III are preferably pharmaceutically acceptable, but it will be appreciated that non-pharmaceutically acceptable salts also fall within the scope of the present disclosure, since these are useful as intermediates in the preparation of pharmaceutically acceptable salts.

The term “pharmaceutically acceptable” may be used to describe any pharmaceutically acceptable salt, hydrate or prodrug, or any other compound which upon administration to a subject, is capable of providing (directly or indirectly) a compound of formula I, II and/or III or an active metabolite or residue thereof.

Suitable pharmaceutically acceptable salts include, but are not limited to, salts of pharmaceutically acceptable inorganic acids such as hydrochloric, sulphuric, phosphoric, nitric, carbonic, boric, sulfamic, and hydrobromic acids, or salts of pharmaceutically acceptable organic acids such as acetic, propionic, butyric, tartaric, maleic, hydroxymaleic, fumaric, malic, citric, lactic, mucic, gluconic, benzoic, succinic, oxalic, phenylacetic, methanesulphonic, toluenesulphonic, benzenesulphonic, salicylic, sulphanilic, aspartic, glutamic, edetic, stearic, palmitic, oleic, lauric, pantothenic, tannic, ascorbic and valeric acids.

Base salts include, but are not limited to, those formed with pharmaceutically acceptable cations, such as sodium, potassium, lithium, calcium, magnesium, zinc, ammonium, alkylammonium such as salts formed from triethylamine, alkoxyammonium such as those formed with ethanolamine and salts formed from ethylenediamine, choline or amino acids such as arginine, lysine or histidine. General information on types of pharmaceutically acceptable salts and their formation is known to those skilled in the art and is as described in general texts such as “Handbook of Pharmaceutical salts” P. H. Stahl, C. G. Wermuth, 1st edition, 2002, Wiley-VCH.

Basic nitrogen-containing groups may be quarternised with such agents as lower alkyl halide, such as methyl, ethyl, propyl, and butyl chlorides, bromides and iodides; dialkyl sulfates like dimethyl and diethyl sulfate; and others.

Prodrugs include compounds wherein an amino acid residue, or a polypeptide chain of two or more (eg, two, three or four) amino acid residues which are covalently joined to free amino, and amido groups of compounds of Formula I, II and/or III. The amino acid residues include the 20 naturally occurring amino acids commonly designated by three letter symbols and also include, 4-hydroxyproline, hydroxylysine, demosine, isodemosine, 3-methylhistidine, norvlin, beta-alanine, gamma-aminobutyric acid, citrulline, homocysteine, homoserine, ornithine and methionine sulfone. Prodrugs also include compounds wherein carbonates, carbamates, amides and alkyl esters which are covalently bonded to the above substituents of Formula I, II and/or III through the carbonyl carbon prodrug sidechain. Prodrugs can include covalent irreversible and reversible inhibitors.

In the case of compounds that are solids, it will be understood by those skilled in the art that the inventive compounds, agents and salts may exist in different crystalline or polymorphic forms, all of which are intended to be within the scope of the present invention and specified formulae.

The term “polymorph” includes any crystalline form of compounds of Formula I, II and/or III, such as anhydrous forms, hydrous forms, solvate forms and mixed solvate forms.

Formula (I), Formula (II) and/or Formula (III) are intended to cover, where applicable, solvated as well as unsolvated forms of the compounds. Thus, Formula (I), Formula (II) and/or Formula (III) include compounds having the indicated structure, including the hydrated or solvated form, as well as the non-hydrated and non-solvated forms.

Pharmaceutical compositions may be formulated from compounds according to Formula (I), Formula (II) and/or Formula (III) for any appropriate route of administration including, for example, topical (for example, transdermal or ocular), oral, buccal, nasal, vaginal, rectal or parenteral administration. The term parenteral as used herein includes subcutaneous, intradermal, intravascular (for example, intravenous), intramuscular, spinal, intracranial, intrathecal, intraocular, periocular, intraorbital, intrasynovial and intraperitoneal injection, as well as any similar injection or infusion technique. In certain embodiments, compositions in a form suitable for oral use or parenteral use are preferred. Suitable oral forms include, for example, tablets, troches, lozenges, aqueous or oily suspensions, dispersible powders or granules, emulsions, hard or soft capsules, or syrups or elixirs. For intravenous, intramuscular, subcutaneous, or intraperitoneal administration, one or more compounds may be combined with a sterile aqueous solution which is preferably isotonic with the blood of the recipient. Such formulations may be prepared by dissolving solid active ingredient in water containing physiologically compatible substances such as sodium chloride or glycine, and having a buffered pH compatible with physiological conditions to produce an aqueous solution, and rendering said solution sterile. The formulations may be present in unit or multi-dose containers such as sealed ampoules or vials. Examples of components are described in Martindale—The Extra Pharmacopoeia (Pharmaceutical Press, London 1993) and Martin (ed.), Remington's Pharmaceutical Sciences.

In the context of this specification the term “administering” and variations of that term including “administer” and “administration”, includes contacting, applying, delivering or providing a compound or composition of the invention to an organism, or a surface by any appropriate means.

For the inhibition of necroptosis, the dose of the biologically active compound according to the invention may vary within wide limits and may be adjusted to individual requirements. Active compounds according to the present invention are generally administered in a therapeutically effective amount. Preferred doses range 5 from about 0.1 mg to about 140 mg per kilogram of body weight per day (e.g. about 0.5 mg to about 7 g per patient per day). The daily dose may be administered as a single dose or in a plurality of doses. The amount of active ingredient that may be combined with the carrier materials to produce a single dosage form will vary depending upon the subject treated and the particular mode of administration. Dosage unit forms will generally contain between about 1 mg to about 500 mg of an active ingredient.

It will be understood, however, that the specific dose level for any particular subject and will depend upon a variety of factors including the activity of the specific compound employed, the age, body weight, general health, sex, diet, time of administration, route of administration, and rate of excretion, drug combination (i.e. other drugs being used to treat the subject), and the severity of the particular disorder undergoing therapy. The dosage will generally be lower if the compounds are administered locally rather than systemically, and for prevention rather than for treatment. Such treatments may be administered as often as necessary and for the period of time judged necessary by the treating physician. A person skilled in the art will appreciate that the dosage regime or therapeutically effective amount of the compound of formula (I) to be administered may need to be optimized for each individual. The pharmaceutical compositions may contain active ingredient in the range of about 0.1 to 2000 mg, preferably in the range of about 0.5 to 500 mg and most preferably between about 1 and 200 mg. A daily dose of about 0.01 to 100 mg/kg body weight, preferably between about 0.1 and about 50 mg/kg body weight, may be appropriate. The daily dose can be administered in one to four doses per day.

It will also be appreciated that different dosages may be required for treating different disorders. An effective amount of an agent is that amount which causes a statistically significant decrease in necroptosis.

For in vitro analysis, the necroptosis inhibition may be determined by assays used to measure TSQ-induced necroptosis, as described in the biological tests defined herein.

The terms “therapeutically effective amount” or “effective amount” refer to an amount of the compound of formula (I) that results in an improvement or remediation of the symptoms of necroptosis and/or associated diseases or their symptoms.

The terms “treating”, “treatment” and “therapy” are used herein to refer to curative therapy, prophylactic therapy and preventative therapy. Thus, in the context of the present disclosure the term “treating” encompasses curing, ameliorating or tempering the severity of necroptosis and/or associated diseases or their symptoms.

“Preventing” or “prevention” means preventing the occurrence of the necroptosis or tempering the severity of the necroptosis if it develops subsequent to the administration of the compounds or pharmaceutical compositions of the present invention.

“Subject” includes any human or non-human animal. Thus, in addition to being useful for human treatment, the compounds of the present invention may also be useful for veterinary treatment of mammals, including companion animals and farm animals, such as, but not limited to dogs, cats, horses, cows, sheep, and pigs.

The term “inhibit” is used to describe any form of inhibition that results in prevention, reduction or otherwise amelioration of necroptosis, including complete and partial inhibition.

The methods of the present disclosure can be used to prevent or treat the following disease in a subject:
diseases of the bones, joints, connective tissue and of cartilage, such as osteoporosis, osteomyelitis, arthritises including for example osteoarthritis, rheumatoid arthritis and psoriatic arthritis, avascular necrosis, progressive fibrodysplasia ossificans, rickets, Cushing's syndrome;
muscular diseases such as muscular dystrophy, such as for example Duchenne's muscular dystrophy, myotonic dystrophies, myopathies and myasthenias;
diseases of the skin, such as dermatitis, eczema, psoriasis, aging or even alterations of scarring;
cardiovascular diseases such as cardiac and/or vascular ischemia, myocardium infarction, ischemic cardiopathy, chronic or acute congestive heart failure, cardiac dysrythmia, atrial fibrillation, ventricular fibrillation, paroxystic tachycardia, congestive heart failure, hypertrophic cardiopathy, anoxia, hypoxia, secondary effects due to therapies with anti-cancer agents;
circulatory diseases such as atherosclerosis (Lin et al., Cell Rep, 2013), arterial scleroses and peripheral vascular diseases, cerebrovascular strokes, aneurisms;
haematological and vascular diseases such as: anemia, vascular amyloidosis, haemorrhages, drepanocytosis, red cell fragmentation syndrome, neutropenia, leukopenia, medullar aplasia, pantocytopenia, thrombocytopenia, haemophilia;
lung diseases including pneumonia, asthma; obstructive chronic diseases of the lungs such as for example chronic bronchitis and emphysema;
diseases of the gastro-intestinal tract, such as ulcers;
diseases of the liver such as for example hepatitis particularly hepatitis of viral origin or having as causative agent other infectious agents, auto-immune hepatitis, fulminating hepatitis, certain hereditary metabolic disorders, Wilson's disease, cirrhoses, non-alcoholic hepatic steatosis, diseases of the liver due to toxins and to drugs such as drug-induced liver injury (Wang et al., Mol Cell, 2014);
diseases of the pancreas such as for example acute or chronic pancreatitis (He et al., Cell, 2009; Zhang et al., Science, 2009; Wu et al., Cell Res, 2013);
metabolic diseases such as diabetes mellitus and insipid diabetes, thyroiditis;
diseases of the kidneys such as for example acute renal disorders or glomerulonephritis;
viral and bacterial infections such as septicemia;
severe intoxications by chemicals, toxins or drugs;
degenerative diseases associated with the Acquired Immune Deficiency Syndrome (AIDS);
disorders associated with aging such as the syndrome of accelerated aging;
inflammatory diseases such as Terminal ileitis (Gunther et al., Nature, 2011; Welz et al., Nature, 2011) including Crohn's disease, rheumatoid polyarthritis, TNF-induced systemic inflammatory syndrome (Duprez et al., Immunity, 2011);
auto-immune diseases such as erythematous lupus;
dental disorders such as those resulting in degradation of tissues such as for example periodontitis;
ophthalmic diseases or disorders including diabetic retinopathies, glaucoma, macular degenerations, retinal degeneration, retinitis pigmentosa, retinal holes or tears, retinal detachment (Trichonas et al., PNAS, 2010), retinal ischemia, acute retinopathies associated with trauma, inflammatory degenerations, post-surgical complications, medicinal retinopathies, cataract, cone cell degeneration (Murakami et al., PNAS, 2012);
disorders of the audition tracts, such as otosclerosis and deafness induced by antibiotics;
Ischemic reperfusion injury (Linkermann et al., Kydney Int, 2012; Oerlemans et al., in vivo Bas Res Cardiol, 2012);
Neuronal loss (Vitner et al., Nat Med, 2014);
diseases associated with mitochondria (mitochondrial pathologies), such as Friedrich's ataxia, congenital muscular dystrophy with structural mitochondrial abnormality, certain myopathies (MELAS syndrome, MERFF syndrome, Pearson's syndrome), MIDD (mitochondrial diabetes and deafness) syndrome, Wolfram's syndrome, dystonia; and
cancer and metastasis including but not limited to cancers of the lung and bronchus, including non-small cell lung cancer (NSCLC), squamous lung cancer, brochioloalveolar carcinoma (BAC), adenocarcinoma of the lung, and small cell lung cancer (SCLC); prostate cancer, including androgen-dependent and androgen-independent prostate cancer; breast cancer, including metastatic breast cancer; pancreatic cancer; cancers of the colon and rectum; thyroid cancer; cancers of the liver and intrahepatic bile duct; hepatocellular cancer; gastric cancer; endometrial cancer; melanoma; cancers of the kidney, renal pelvis, urinary bladder, uterine corpus and uterine cervix; ovarian cancer, including progressive epithelial or primary peritoneal cancer; multiple myeloma; oesophageal cancer, including squamous cell carcinoma and adenocarcinoma of the oesophagus; acute myelogenous leukemia (AML); chronic myelogenous leukemia (CML), including accelerated CML and CML blast phase (CML-BP); lymphocytic leukemia; myeloid leukemia; acute lymphoblastic leukemia (ALL); chronic lymphocytic leukemia (CLL); Hodgkin's disease (HD); non-Hodgkin's lymphoma (NHL), including follicular lymphoma and mantle cell lymphoma; B-cell lymphoma, including diffuse large B-cell lymphoma (DLBCL); T-cell lymphoma; multiple myeloma (MM); amyloidosis; Waldenstrom's macroglobulinemia; myelodysplastic syndromes (MDS), including refractory anemia (RA), refractory anemia with ringed siderblasts (RARS), (refractory anemia with excess blasts (RAEB), and RAEB in transformation (RAEB-T); and myeloproliferative syndromes; cancers of the brain, including glioma/glioblastoma, anaplastic oligodendroglioma, and adult anaplastic astrocytoma; neuroendocrine cancers, including metastatic neuroendocrine tumors; cancers of the head and neck, including, e.g., squamous cell carcinoma of the head and neck, and nasopharyngeal cancer; cancers of the oral cavity, pharynx and small intestine; bone cancer; soft tissue sarcoma; and villous colon adenoma.

The methods can also be used for protecting cells, tissues and/or transplanted organs, whether before, during (removal, transport and/or re-implantation) or after transplantation.

In one embodiment, in the methods of the present disclosure the subject is not suffering from a proliferative disorder associated with excessive angiogenesis. In a further embodiment, the proliferative disorder is cancer.

The present disclosure also provides screening methods for identifying a compound which inhibits necroptosis by binding to the ATP-binding site of the pseudokinase domain of MLKL. Accordingly, in another aspect, the present disclosure provides a screening method for identifying a compound which inhibits necroptosis, the method comprising:
a) contacting a protein solution containing MLKL with a candidate compound under conditions allowing the interaction of MLKL and the candidate compound; and
b) comparing the unfolding transition temperature (Tₘ) obtained in the presence of the candidate compound with the unfolding transition temperature (Tₘ) obtained in the absence of the candidate compound to determine the change in the unfolding transition temperature (ΔTₘ);
wherein the interaction of MLKL pseudokinase domain and the candidate compound is through binding of the candidate compound to the ATP-binding site of the pseudokinase domain of MLKL; and
wherein a positive ΔTₘ value indicates that the candidate compound stabilizes the protein from denaturation and inhibits its role in necroptosis.

In one embodiment, the screening method includes a protein solution which comprises a fluorescent dye. The fluorescent dye may be any dye suitable for such a use as determined by a person skilled in the art. In one embodiment, the fluorescent dye is SYPRO Orange.

The screening method may be performed on any instrument known to be suitable for such a use as determined by a person skilled in the art. In one embodiment, the instrument combines sample temperature control and dye fluorescence detection. In another embodiment, the instrument is a real-time polymerase chain reaction (RT-PCR) machine.

In another aspect, there is provided a screening method for identifying a compound which inhibits necroptosis, the method comprising:
a) contacting an MLKL pseudokinase domain with increasing concentrations of a candidate compound under conditions allowing the interaction of MLKL pseudokinase domain and the candidate compound; and
b) determining the binding affinity (K_{d});
wherein the interaction of MLKL pseudokinase domain and the candidate compound is through binding of the candidate compound to the ATP-binding site of the pseudokinase domain of MLKL, and
wherein binding of the candidate compound indicates that the candidate compound is capable of inhibiting necroptosis.

The screening method may be performed in any way known to be suitable for such a use as determined by a person skilled in the art. Further, the screening method may be performed on any instrument known to be suitable for such a use as determined by a person skilled in the art. In one embodiment, the screening method includes SPR and is performed on an SPR machine. In another embodiment, the screening method includes the use of the MLKL pseudokinase domain immobilised on a sensor chip. In a further embodiment, the immobilised MLKL pseudokinase domain is double His-tagged. In yet another embodiment, the screening method includes SPR and is performed on an SPR machine and involves the use of the MLKL pseudokinase domain immobilised on a sensor chip.

In the present disclosure, the term “under conditions allowing the interaction of MLKL pseudokinase domain and the candidate compound” includes all conditions under which such interaction is possible. Such conditions would be known or readily determined by a person skilled in the art.

In another aspect, there is provided a screening method for identifying a compound which inhibits necroptosis, the method comprising:
a) contacting a protein solution containing MLKL pseudokinase domain with a nucleotide and a candidate compound and performing STD-NMR; and
b) comparing the STD-NMR spectrum obtained in the presence of the candidate compound and the STD-NMR spectrum obtained in the absence of the candidate compound;
wherein the interaction of MLKL and the candidate compound is through binding of the candidate compound to the ATP-binding site of the pseudokinase domain of MLKL; and
wherein the disappearance or reduction of the signal intensity in the STD-NMR spectrum indicates that the candidate compound is capable of inhibiting necroptosis.

The screening method may be performed in any way known to be suitable for such a use as determined by a person skilled in the art.

In one embodiment, the screening method includes the use of a nucleotide selected from the group consisting of AMP, AMPPNP, ADP and ATP. In another embodiment, the screening method includes the use of a nucleotide selected from the group consisting of ATP and ADP. In another embodiment, the nucleotide is ATP. In another embodiment, the nucleotide is ADP.

In the present disclosure, the term “protein solution” includes any solution which contains the MLKL protein, or part thereof, in a suitable manner to allow for the screening method to be performed. A suitable “protein solution” would be known or readily determined by a person skilled in the art. In one embodiment, the “protein solution” may include additional components. The additional components include any component that is required by the screening method employed.

The screening methods of the present disclosure may be carried out with MLKL from any species. In one embodiment, the MLKL is mouse MLKL as defined hereinbefore. In another embodiment, the MLKL is human MLKL as defined hereinbefore.

The screening methods of the present disclosure may also include cell death assays to determine the ability of the candidate compound to inhibit necroptosis in cells. The cell death assays may be carried out on cell or tissue cultures. Cells used in this screening method may be any cells that can express MLKL, irrespective of the difference between natural and recombinant genes. Moreover, the derivation of the MLKL is not particularly limited. Moreover, transformed cells that contain expression vectors comprising nucleic acid sequences that encode MLKL may also be used.

The cells may be human or non-human cells. In one embodiment, the cells are human cells. In another embodiment, the cells are non-human cells. In yet another embodiment, the cells are non-human mammalian cells.

Examples of suitable human cells, include cancer and non-cancer cells. In one embodiment, the human cells are a non-cancer cell line. In another embodiment, the human cells are a cancer cell line. In one embodiment, the human cells are a leukemia cell line. The leukemia cell line may be selected from, but not limited to, the group comprising acute myeloid leukemia (AML), myelomonocytic leukemia and T cell leukemia. The leukaemia cell line may be selected from, but not limited to, the group comprising Jurkat T cells, FADD-deficient variant of Jurkat cells, Mv4;11 cells, OCI-AML-3cells and U937 cells. In one embodiment, the human cells are U937 cells. In another embodiment, the human cells are a human colon adenocarcinoma cell line. In one embodiment, the human colon adenocarcinoma cell line is HT29.

Suitable non-human mammalian cells include, but are not limited to, cells obtained from rodents (for example, mice, rats, hamsters and guinea pigs), rabbits, equines (for example, horses), canines (for example, dogs), felines (for example, cats), bovines (for example, cows), ovines (for example, sheep and goats), primates (for example, monkeys), avians (for example, chickens), or the like. Examples of the non-human mammalian cells and avian cells include culture cells such as, CHO cells, NIH3T3 cells, COS cells, DT40 cells, BHK cells, MDCK cells, CRFK cells, CV-1 cells, LMTK cells and Vero cells; primary culture cells; hematopoietic stem cells; hematopoietic cells and blood cells such as B cells, T cells, thymocytes, white blood cells, monocytes and macrophages, red blood cells, and platelets; fertilized oocytes; and ES cells. Further, other cells such as various tissue, kidney, fibroblast and myeloma cells may also be used. In one embodiment, the cells are rodent cells. In another embodiment, the cells are mouse cells. In another embodiment, the cells are mouse dermal fibroblasts (MDFs).

Accordingly, in one embodiment the present disclosure provides a screening method as described herein which further comprises testing the ability of the candidate compound to rescue cell death by necroptosis. In one embodiment, the testing is performed in an in vitro cell based assay. In a further embodiment, the cells are mouse dermal fibroblasts (MDFs).

The cell death assays may be performed in any way known to be suitable as determined by a person skilled in the art. In one embodiment, the cell death assays include the determination of propidium iodide (PI) uptake using flow cytometry, in the absence or presence of the necroptotic stimulus.

In the cell death assays of the present disclosure, necroptosis may be induced in the cell in any way known to be suitable as determined by a person skilled in the art. In one embodiment, necroptosis is stimulated by a factor selected from any one or more of the following: TNF (T); Smac mimetic (S); and the pan-caspase inhibitor Q-VD-OPh (Q). In another embodiment, the cell death assay includes a necroptotic stimulus of TNF (T), Smac mimetic (S) and the pan-caspase inhibitor Q-VD-OPh (Q), this combination of necroptotic stimuli being termed “TSQ”.

The methods and compounds described herein are described by the following illustrative and non-limiting examples.

### EXAMPLES

1.1 Materials and Methods

Compounds.

Compound 1 was identified in a screen of a commercial library.

All temperatures referred to are in ° C.

The names of the following compounds have been obtained using ChemDraw Ultra 12.0.

Abbreviations

AcOH acetic acid

(Boc)₂O di-tert-butyl dicarbonate

CDCl₃ deuterochloroform

CDI 1,1′-Carbonyldiimidazole

Cs₂C0₃ caesium carbonate

DMSO-d₆ deuterated dimethylsulfoxide

DCC dicyclohexylcarbodiimide

DCM dichloromethane

DIPEA diisopropylethylamine

DMF N,N-dimethylformamide

DMSO dimethylsulfoxide

TEA triethylamine

EtOAc ethylacetate

EtOH ethanol

hr hour(s)

HATU 1-[Bis(dimethylamino)methylene]-1H-1,2,3-triazolo[4,5-b]pyridinium 3-oxide hexafluorophosphate

HCl hydrochloric acid/hydrogen chloride

HPLC high performance liquid chromatography

K₂CO₃ potassium carbonate

LCMS liquid chromatography-mass spectrometry

M molar (concentration)

MeOH methanol

min minute(s)

M/Z mass/charge ratio (mass spectrometry)

Na₂CO₃ sodium carbonate

NaH sodium hydride

NaHCO₃ sodium bicarbonate

NaOH sodium hydroxide

Na₂SO₄ sodium sulphate

NH₄Cl ammonium chloride

NMP N-methyl-2-pyrrolidinone

NMR nuclear magnetic resonance

Pd/C palladium on activated charcoal

Rt retention time

rt room temperature

SOCl₂ thionyl chloride

TFA trifluoroacetic acid

THF tetrahydrofuran

TsOH tosyl chloride

LCMS Methodology

Electrospray mass spectroscopy (MS) was carried out using the following method;

Method A(10 min method): Finnigan LCQ Advantage Max using reverse phase high performance liquid chromatorgraphy (HPLC) analysis (column: Gemini 3μ C18 20×4.0 mm 110A) Solvent A: Water 0.1% Formic Acid, Solvent B: Acetonitrile 0.1% Formic Acid, Gradient: 10-100% B over 10 min Detection: 100-600 nm using electrospray ionisation (ESI) positive mode with source temperature 300° C.

Method B (5 min method): LC model: Agilent 1200 (Pump type: Binary Pump, Detector type: DAD) MS model: Agilent G6110A Quadrupole. Column: Xbridge-C18, 2.5 μm, 2.1×30 mm. Column temperature: 30° C. Acquisition of wavelength: 214 nm, 254 nm. Mobile phase: A: 0.07% HCOOH aqueous solution, B: MeOH. Run time: 5 min. MS: Ion source: ES+(or ES−). MS range: 50˜900 m/z. Fragmentor: 60. Drying gas flow: 10 L/min. Nebulizer pressure: 35 psi. Drying gas temperature: 350° C. Vcap: 3.5 kV.

Preparative Mass-Directed LC

Method A:

Instrument:

Waters ZQ 3100-Mass Detector, Waters 2545-Pump, Waters SFO System Fluidics Organizer, Waters 2996 Diode Array Detector, Waters 2767 Sample Manager

LC Conditions:

Reverse Phase HPLC analysis

Column: XBridge™ C18 5 μm 19×50 mm. Injection Volume 500 μL

Solvent A: Water 0.1% Formic Acid. Solvent B:MeCN 0.1% Formic Acid

Gradient: 5% B over 4 min then 5-100% B over 8 min then 100% B over 4 min

Flow rate: 19 mL/min. Detection: 100-600 nm

MS Conditions:

Ion Source: Single-quadrupole. Ion Mode: ES positive. Source Temp: 150° C.

Desolvation Temp: 350° C. Detection: Ion counting. Capillary (KV)-3.00. Cone (V): 30

Extractor (V): 3 RF Lens (V): 0.1 Scan Range: 100-1000 Amu Scan Time: 0.5 sec

Acquisition time: 10 min

Gas Flow:
Desolvation L/hour-650
Cone L/hour-100

Preparative HPLC

Instrument type:

VARIAN 940 LC. Pump type: Binary Pump. Detector type: PDA

LC Conditions:

Column: Waters SunFire prep C18 OBD, 5 μm, 19×100 mm. Acquisition wavelength: 214 nm, 254 nm. Mobile Phase: A: 0.07% TFA aqueous solution, B: MeOH, 0.07% TFA.

NMR

Nuclear magnetic resonance (¹H NMR, 600 MHz or 400 MHz) spectra were obtained at 300 K with the CDCl₃ as the solvent, unless otherwise indicated. Chemical shifts are reported in ppm on the δ scale and referenced to the appropriate solvent peak.

### Synthesis of Intermediate A

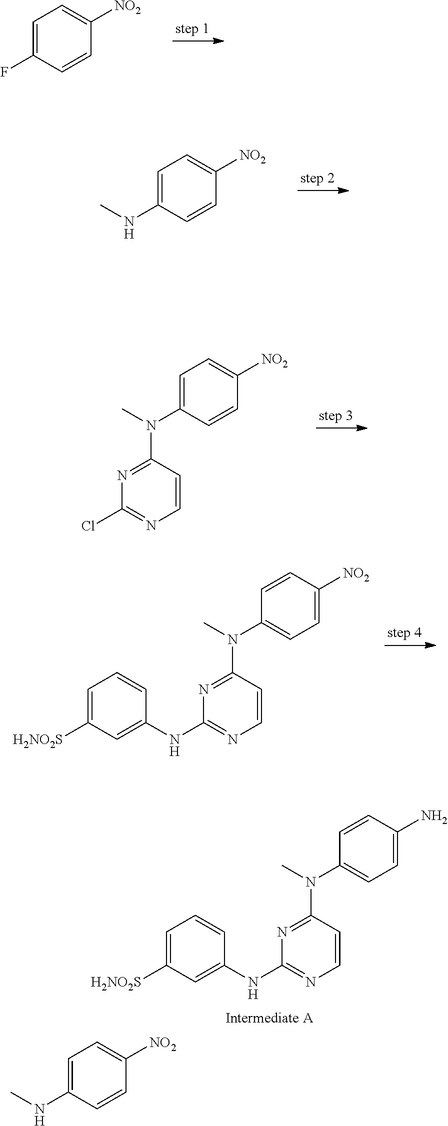 [SMILES: O=[N+]([O-])c1ccc(F)cc1 ; CNc1ccc([N+](=O)[O-])cc1 ; CN(c1ccc([N+](=O)[O-])cc1)c1ccnc(Cl)n1 ; CN(c1ccc([N+](=O)[O-])cc1)c1ccnc(Nc2cccc(S(N)(=O)=O)c2)n1 ; CN(c1ccc(N)cc1)c1ccnc(Nc2cccc(S(N)(=O)=O)c2)n1 ; CNc1ccc([N+](=O)[O-])cc1]

### Step 1: N-methyl-4-nitrobenzenamine

To a solution of 1-fluoro-4-nitrobenzene (50.0 g, 354 mmol) in DMSO (200 mL) were added methanamine hydrochloride (47.1 g, 709 mmol) and potassium carbonate (98.0 g, 709 mmol). The resulting mixture was stirred overnight at 70° C. under nitrogen atmosphere. TLC analysis indicated that the reaction was complete. The mixture was poured into water to give a precipitate which was filtered off and then washed with additional water and dried to yield desired product as yellow solid (50 g, 93%). LCMS (Method B): 1.63 min [MH]⁺=153.1, [MNa]⁺=175.1.

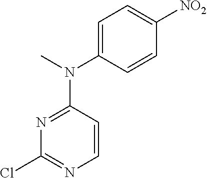 [SMILES: CN(c1ccc([N+](=O)[O-])cc1)c1ccnc(Cl)n1]

### Step 2: 2-chloro-N-methyl-N-(4-nitrophenyl)pyrimidin-4-amine

To a solution of N-methyl-4-nitrobenzenamine (30.0 g, 197 mmol) in DMF (150 mL) was added 2,4-dichloropyrimidine (29.4 g, 197 mmol) and potassium carbonate (40.88 g, 296 mmol). The resulting mixture was stirred at 80° C. overnight. The mixture was diluted with ethyl acetate (300 mL) and water (200 mL), and the combined organic phases were washed with water, brine, dried over sodium sulfate and concentrated to give a residue, which was purified by column chromatography on silica gel (petroleum ether/ethyl acetate, 10:1 to 3:1) to give the desired product (20 g, 38%) as a yellow solid. LCMS (Method B): 2.34 min [MH]⁺=265.0, 267.0, [MNa]⁺=287.0, 289.0.

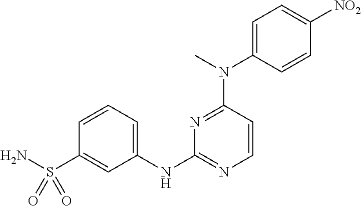 [SMILES: CN(c1ccc([N+](=O)[O-])cc1)c1ccnc(Nc2cccc(S(N)(=O)=O)c2)n1]

### Step 3: 3-((4-(methyl(4-nitrophenyl)amino)pyrimidin-2-yl)amino) benzenesulfonamide

To a solution of 2-chloro-N-methyl-N-(4-nitrophenyl)pyrimidin-4-amine (1.0 g, 3.8 mmol) in 1,4-dioxane (10 mL) was added 3-aminobenzenesulfonamide (651 mg, 3.8 mmol) and conc HCl (0.5 mL). The resulting mixture was stirred at 160° C. for two hours under microwave. LCMS and TLC analysis indicated that the reaction was complete. The solvent was removed under reduced pressure to give a residue. The residue was dissolved into NaOH aqueous solution (4M) and DCM. The organic layer was washed with water, brine, dried over sodium sulfate and concentrated to give a residue, which was purified by column chromatography on silica gel (DCM/MeOH, 30:1 to 10:1) to give the desired product (1 g, 67%) as a yellow solid. LCMS (Method B): 0.96 min [MH]⁺=401.1.

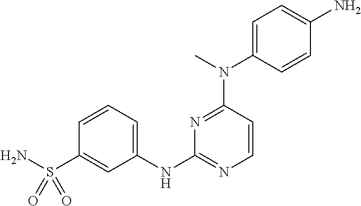 [SMILES: CN(c1ccc(N)cc1)c1ccnc(Nc2cccc(S(N)(=O)=O)c2)n1]

### Step 4: (Intermediate A) 3-((4-(methyl(4-(3-(4-(trifluoromethoxy) phenyl)ureido)phenyl)amino)pyrimidin-2-yl)amino)benzenesulfonamide

To a solution of 3-((4-(methyl(4-nitrophenyl)amino)pyrimidin-2-yl)amino)benzene sulfonamide (800 mg, 2.0 mmol) in EtOH (20 mL) was added zinc (1.3 g, 20 mmol) and NH₄Cl (aq 30 ml). The reaction mixture was stirred at 90° C. for two hours. LCMS and TLC analysis indicated that the reaction was complete. The mixture was filtered off and the liquid was concentrated in vacuo to remove EtOH then the mixture was filtered off to give the desired product (320 mg, 43%) as a white solid. ¹H NMR (400 MHz, DMSO-d₆): δ ppm 3.34 (s, 3H) 5.22 (s, 2H) 5.68 (d, J=6.0 Hz, 1H) 6.65 (d, J=8.4 Hz, 2H) 6.95 (d, J=8.4 Hz, 2H) 7.23 (s, 2H) 7.41 (m, 2H) 7.81 (m, 2H) 8.57 (s, 1H) 8.42 (s, 1H). LCMS (Method B): 0.37 min [MH]⁺=370.1

### Synthesis of Intermediate B

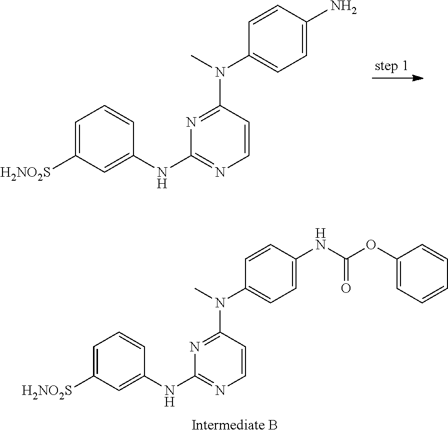 [SMILES: CN(c1ccc(N)cc1)c1ccnc(Nc2cccc(S(N)(=O)=O)c2)n1 ; CN(c1ccc(NC(=O)Oc2ccccc2)cc1)c1ccnc(Nc2cccc(S(N)(=O)=O)c2)n1]

### Step 1: (intermediate B) 3-(4-(methyl(4-(3-phenylureido)phenyl)amino) pyrimidin-2-ylamino)benzenesulfonamide

To a bottom flask, 3-((4-((4-aminophenyl)(methyl)amino)pyrimidin-2-yl)amino) benzene sulfonamide (intermediate A (1.5 g, 4.1 mmol) was dissolved in THF (20 mL). Pyridine (320 mg, 12.2 mmol) was then added. Phenylchloroformate (698 mg, 4.5 mmol) was added to the mixture slowly. The reaction mixture was stirred at room temperature overnight. The solvent was removed. The crude was washed with water (2×50 mL), diethyl ether (2×50 mL) and dried to give phenyl(4-(methyl(2-((3-sulfamoylphenyl)amino)pyrimidin-4-yl)amino)phenyl) carbamate (1.5 g, 76%) as a yellow solid. ¹H NMR (400 MHz, MeOD-d₄): δ ppm 3.54 (s, 3H), 5.87 (d, J=6.4 Hz, 1H), 7.28 (m, 5H), 7.45 (m, 5H), 7.68 (m, 4H), 7.82 (d, J=6.4 Hz, 1H), 8.62 (br s, 1H).

LCMS (Method B): 2.17 [M+H]+=491.2.

### Synthesis of Intermediate C

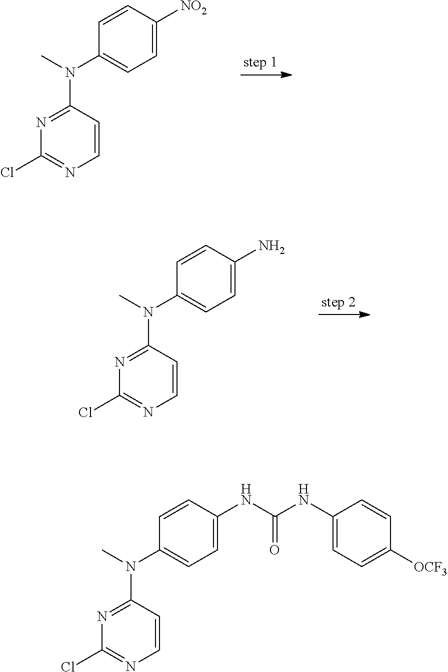 [SMILES: CN(c1ccc([N+](=O)[O-])cc1)c1ccnc(Cl)n1 ; CN(c1ccc(N)cc1)c1ccnc(Cl)n1 ; CN(c1ccc(NC(=O)Nc2ccc(OC(F)(F)F)cc2)cc1)c1ccnc(Cl)n1]

### Step 1: N1-(2-chloropyrimidin-4-yl)-N1-methylbenzene-1,4-diamine

2-Chloro-N-methyl-N-(4-nitrophenyl)pyrimidin-4-amine (from step 2 of preparation of intermediate A, 100 mg, 0.38 mmol) was dissolved in methanol (10 mL) and aq. NH₄Cl (10 mL). Zinc (powder, 245 mg, 3.0 mmol) was added. The reaction mixture was stirred at room temperature overnight. The mixture was concentrated under reduced pressure.

The residue was extracted with EtOAc (3×20 mL). The combined organic layers were washed with brine (20 mL), dried over Na₂SO₄. The solvent was removed under reduced pressure to give N1-(2-chloropyrimidin-4-yl)-N1-methylbenzene-1,4-diamine (80 mg, 90%) as a yellow solid which was used in next step directly. LCMS (Method B): 1.10 [M+H]⁺=235.1

### Step 2: (Intermediate C) 1-(4-((2-chloropyrimidin-4-yl) (methyl)amino)phenyl)-3-(4-(trifluoromethoxy)phenyl)urea

To a bottom flask, N1-(2-chloropyrimidin-4-yl)-N1-methylbenzene-1,4-diamine (776 mg, 3.31 mmol) was dissolved in DCM (4 mL). 1-isocyanato-4-(trifluoromethoxy)benzene (672 mg, 3.31 mmol) was added to the mixture. The reaction mixture was stirred at room temperature overnight. The white solid was filtered off and washed with DCM (20 mL), and dried to give 1-(4-((2-chloropyrimidin-4-yl) (methyl)amino)phenyl)-3-(4-(trifluoromethoxy)phenyl)urea (1.06 g, 73%) as a white solid. ¹H NMR (400 MHz, DMSO-d₆): δ ppm 3.40 (s, 3H) 6.27 (d, J=6.0 Hz, 1H) 7.31 (m, 4H) 7.60 (m, 4H) 7.98 (d, J=6.0 Hz, 1H) 8.90 (s, 1H) 8.93 (s, 1H). LCMS (Method B): 3.09 [M+H]⁺=438.2.

General Procedure A for the Synthesis of the Ureas:

To a solution of the intermediate A (1 mmol) in dry DMF (0.2 mL) under N₂ was added an isocyanate (1 mmol) dropwise. The reaction mixture was stirred for 16 hours at rt, concentrated under reduced pressure and the residue was purified by preparative mass directed LC to afford the corresponding compound.

General Procedure B for the Synthesis of the Ureas:

To a solution of intermediate B (1 mmol) in dry THF under N₂ were added an arylamine (2 mmol) followed by DIEA (2 mmol). The reaction mixture was heated to 60° C. for 16 hours, concentrated under reduced pressure and the residue was purified by preparative HPLC to afford the corresponding compound.

General Procedure C for the Addition of Aniline to 2-Chloropyrimidine:

To a solution of aniline (1 mmol) and intermediate C (1 mmol) in 2-propanol (10 mL) was added a solution of concentrated HCl (2 drops). The reaction mixture was heated to 80° C. for 16 hours. The solvent was removed and the crude product was purified by column chromatography to afford the corresponding compound.

Compound 1

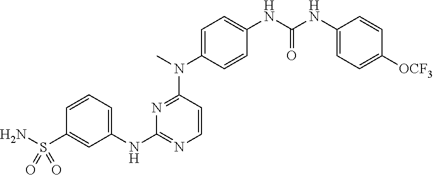 [SMILES: CN(c1ccc(NC(=O)Nc2ccc(OC(F)(F)F)cc2)cc1)c1ccnc(Nc2cccc(S(N)(=O)=O)c2)n1]

3-((4-(methyl(4-(3-(4-(trifluoromethoxy)phenyl)ureido)phenyl)amino)pyrimidin-2-yl)amino)benzenesulfonamide

Following general procedure A using intermediate A (100 mg, 0.27 mmol) and (4-trifluoromethoxyphenyl)isocyanate (55 mg, 0.27 mmol), 3-((4-(methyl(4-(3-(4-(trifluoromethoxy)phenyl)ureido)phenyl)amino)pyrimidin-2-yl)amino)benzenesulfonamide (17 mg, 12%) was obtained as a white solid. ¹H NMR (400 MHz, DMSO-d₆): δ ppm 3.50 (s, 3H), 5.96 (br s, 1H), 7.21 (m, 1H), 7.32 (m, 3H), 7.40 (s, 2H), 7.63 (m, 7H), 7.90 (d, J=7.20 Hz, 1H), 8.37 (br s, 1H), 9.33 (m, 2H), 10.66 (br s, 1H). LCMS (Method B): 2.42 min [MH]⁺=574.1.

Compound 2

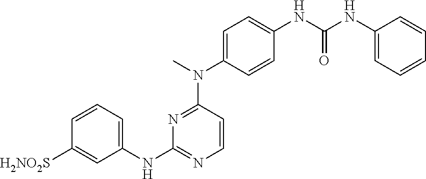 [SMILES: CN(c1ccc(NC(=O)Nc2ccccc2)cc1)c1ccnc(Nc2cccc(S(N)(=O)=O)c2)n1]

### 3-(3-(Methyl(4-(3-phenylureido)phenyl)amino)phenylamino)benzene-sulfonamide

Following general procedure A using intermediate A (50 mg, 0.135 mmol) and phenylisocyanate, 3-(3-(Methyl(4-(3-phenylureido)phenyl)amino)phenylamino) benzene sulfonamide (2.5 mg, 2%) was obtained as a white solid. ¹H NMR (600 MHz, MeOD-d₄): δ ppm 3.51 (s, 3H), 5.92 (s, 1H), 7.02 (m, 1H), 7.23 (d, J=8.7 Hz, 2H), 7.25-7.30 (m, 2H), 7.41-7.44 (m, 3H), 7.52 (m, 1H), 7.56 (d, J=8.7 Hz, 2H), 7.67 (d, J=8.5 Hz, 1H), 7.96 (s, 1H), 8.58 (s, 1H). LCMS (Method A): 4.10 min [MH]⁺=490.4.

Compound 3

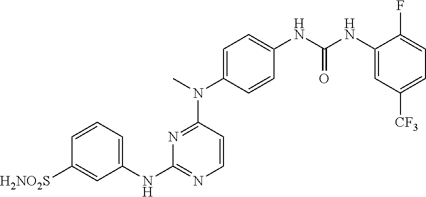 [SMILES: CN(c1ccc(NC(=O)Nc2cc(C(F)(F)F)ccc2F)cc1)c1ccnc(Nc2cccc(S(N)(=O)=O)c2)n1]

### 3-(3-((4-(3-(2-fluoro-5-(trifluoromethyl)phenyl)ureido)phenyl)(methyl)amino) phenylamino) benzenesulfonamide

Following general procedure A using intermediate A (50 mg, 0.135 mmol) and 2-fluoro-5-trifluoromethylphenylisocyanate (10.9 mg, 0.135 mmol), 3-(3-((4-(3-(2-fluoro-5-(trifluoromethyl)phenyl)ureido)phenyl)(methyl)amino)phenylamino) benzenesulfonamide (9 mg, 7%) as a white solid. ¹H NMR (600 MHz, MeOD-d₄): δ ppm 3.52 (s, 3H), 5.88 (m, 1H), 7.26 (d, J=9.0 Hz, 2H), 7.33 (d, J=9.0 Hz, 1H), 7.42 (m, 1H), 7.51 (m, 1H), 7.60 (d, J=9.0 Hz, 1H), 7.68 (m, 1H), 7.76 (br d, J=7.8 Hz, 1H), 8.17 (m, 1H), 8.57 (s, 1H), 8.60 (m, 1H). LCMS (Method A): 4.60 min [MH]⁺=576.3.

Compound 4

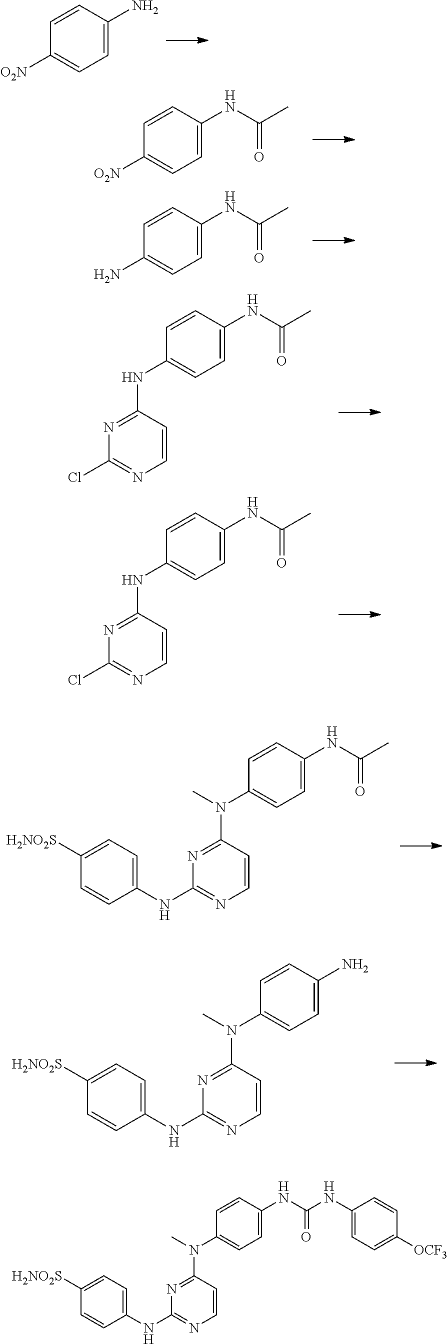

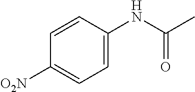

### Step 1: N-(4-Nitrophenyl)acetamide

To a solution of 4-nitroaniline (5.0 g, 36.2 mmol) in EtOAc (70 mL) at 0° C. was added acetic anhydride (3.77 mL, 39.8 mmol) dropwise. The reaction mixture was stirred at room temperature for 16 hours and concentrated under reduced pressure to afford the titled compound as a white solid (6.36 g, 97%). ¹H NMR (600 MHz, MeOD-d₄): δ ppm 2.15 (s, 3H), 7.80 (d, 2H), 8.19 (d, 2H).

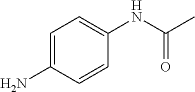

### Step 2: N-(4-Aminophenyl)acetamide

Sodium borohydride (2.87 g, 75.9 mmol) was dissolved in a mixture methanol/H₂O (180 mL/90 mL) and cooled to 0° C. Pd/C (0.443 mg, 10 mol %) was added by small portion with a stream of N₂, followed by N-(4-nitrophenyl)acetamide (4.55 g, 25.3 mmol) in MeOH (3 mL). The reaction mixture was stirred at room temperature for 72 hours, filtered through a pad of celite and rinsed three times with MeOH. The combined filtrates were reduced in vacuo, and partitioned between EtOAc/H₂O. The organic layer was separated and the aqueous layer was extracted twice with EtOAc. The combined organic layers were washed with brine, dried over MgSO₄ and concentrated under reduced pressure to afford the titled compound (4.39 g, 80%) as a light yellow solid. ¹H NMR (600 MHz, DMSO-d₆): δ ppm 1.95 (s, 3H), 4.80 (br s, 2H), 6.48 (d, 2H), 7.18 (d, 2H), 9.46 (s, 1H). LCMS (Method A): 0.82 min [MH]⁺=151.3.

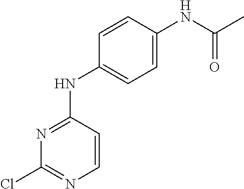 [SMILES: CC(=O)Nc1ccc(Nc2ccnc(Cl)n2)cc1]

### Step 3: N-(4-(2-Chloropyrimidin-4-ylamino)phenyl)acetamide

N-(4-aminophenyl)acetamide (3.91 g, 26.0 mmol) was dissolved in a mixture EtOH/THF (345 mL/115 mL) and the solution was cooled to 0° C. in an ice bath. 2,4-dichloropyrimidine (4.65 g, 31.2 mmol) was added portionwise, followed by NaHCO₃ (4.37 g, 52.0 mmol). The reaction mixture was stirred at room temperature for 16 hours, and concentrated under reduced pressure. EtOAc was added, the organic layer was separated and the aqueous layer was extracted twice with EtOAc. The combined organic layers were washed with brine, dried over MgSO₄ and concentrated in vacuo to afford the titled compound as a crude solid (6.83 g). ¹H NMR (600 MHz, DMSO-d₆): δ ppm 2.01 (s, 3H), 6.66 (d, J=8.7 Hz, 1H), 7.42-7.54 (m, 4H), 8.08 (d, J=9.0 Hz 1H), 9.92 (s, 2H). LCMS (Method A): 4.88 min [MH]⁺=263.2.

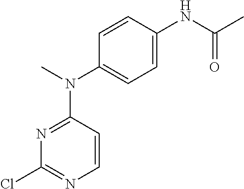 [SMILES: CC(=O)Nc1ccc(N(C)c2ccnc(Cl)n2)cc1]

### Step 4: N-(4-((2-chloropyrimidin-4-yl)(methyl)amino)phenyl)acetamide

N-(4-(2-chloropyrimidin-4-ylamino)phenyl)acetamide (6.83 g, 26.0 mmol) was dissolved in DMF (150 mL) under N₂ and K₂CO₃ (5.39 g, 39.0 mmol) was added, followed by MeI (1.78 mL, 28.6 mmol) dropwise. The reaction mixture was stirred at room temperature for 72 hours and partitioned between EtOAc and water (350 mL/350 mL). The organic layer was washed with brine, dried over MgSO₄ and concentrated in vacuo. The residue was purified by flash column chromatography (eluent: DCM/MeOH 98/2) to afford the titled compound (2.49 g, 34% over 2 steps) as a light yellow solid. ¹H NMR (600 MHz, DMSO-d₆): δ ppm 2.03 (s, 3H) 3.32 (s, 3H) 6.21 (m, 1H) 7.24 (d, 2H) 7.66 (d, 2H) 7.93 (m, 1H) 10.08 (s, 1H). LCMS (acidic 10 min): 4.70 min [MH]⁺=413.3

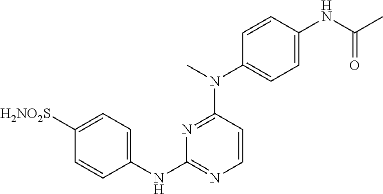 [SMILES: CC(=O)Nc1ccc(N(C)c2ccnc(Nc3ccc(S(N)(=O)=O)cc3)n2)cc1]

### Step 5: N-(4-(Methyl(2-(4-sulfamoylphenylamino)pyrimidin-4-yl)amino)phenyl) acetamide

N-(4-((2-chloropyrimidin-4-yl)(methyl)amino)phenyl)acetamide (1.34 g, 4.84 mmol) and sulfanilamide (0.84 g, 4.84 mmol) were dissolved in i-PrOH (45 mL). Concentrated HCl (10 drops) was added dropwise to the stirred solution. The reaction mixture was stirred for 40 hours at 80° C. and cooled to room temperature. The off-white precipitate formed was collected by filtration, washed with EtOAc and dried under vacuum to afford the titled compound as a white solid (1.54 g, 77%). ¹H NMR (600 MHz, DMSO-d₆): δ ppm 2.06 (s, 3H), 3.48 (s, 3H), 6.65 (s, 1H), 7.27-7.35 (m, 6H), 7.46 (m, 2H), 7.73 (d, J=8.7 Hz, 2H), 10.26 (br s, 1H). LCMS (Method A): 2.97 min [MH]⁺=413.3.

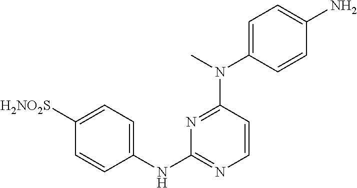 [SMILES: CN(c1ccc(N)cc1)c1ccnc(Nc2ccc(S(N)(=O)=O)cc2)n1]

### Step 6: 4-(4-((4-Aminophenyl)(methyl)amino)pyrimidin-2-ylamino)benzene-sulfonamide

N-(4-(methyl(2-(4-sulfamoylphenylamino)pyrimidin-4-yl)amino)phenyl) acetamide (1.54 g, 3.73 mmol) was dissolved in MeOH (40 mL). Acetyl chloride (2.65 mL, 37.3 mmol) was added dropwise and the reaction mixture was stirred at room temperature for 48 hours and concentrated in vacuo to afford the titled compound (1.40 g, 100%) as a white solid. ¹H NMR (600 MHz, DMSO-d₆): δ ppm 3.48 (s, 3H), 6.72 (d, J=8.7 Hz, 2H), 7.32 (m, 2H), 7.39 (m, 2H), 7.77 (m, 2H). LCMS (Method A): 0.72 min [MH]⁺=371.2.

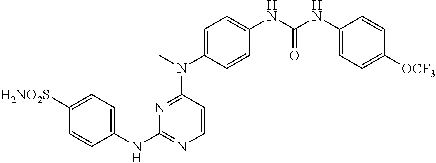 [SMILES: CN(c1ccc(NC(=O)Nc2ccc(OC(F)(F)F)cc2)cc1)c1ccnc(Nc2ccc(S(N)(=O)=O)cc2)n1]

### Step 7: 4-(4-(Methyl(4-(3-(4-(trifluoromethoxy)phenyl)ureido)phenyl)amino)pyrimidin-2-ylamino)benzenesulfonamide

Following general procedure A using 4-(4-((4-Aminophenyl)(methyl)amino)pyrimidin-2-ylamino)benzenesulfonamide (100 mg, 0.27 mmol) and (4-trifluoromethoxyphenyl) isocyanate (0.27 mmol), the titled compound (18 mg, 14%) was obtained as a white solid. ¹H NMR (600 MHz, MeOD-d₄): δ ppm 3.47 (s, 3H), 5.96 (br d, J=5.4 Hz, 1H), 7.20 (d, J=9.0 Hz, 1H), 7.24 (d, J=9.0 Hz, 1H), 7.53 (d, J=9.0 Hz, 2H), 7.57 (d, J=9.0 Hz, 2H), 7.73-7.77 (m, 4H), 7.84 (m, 1H). LCMS (Method A): 4.64 min [MH]⁺=574.4.

Compound 5

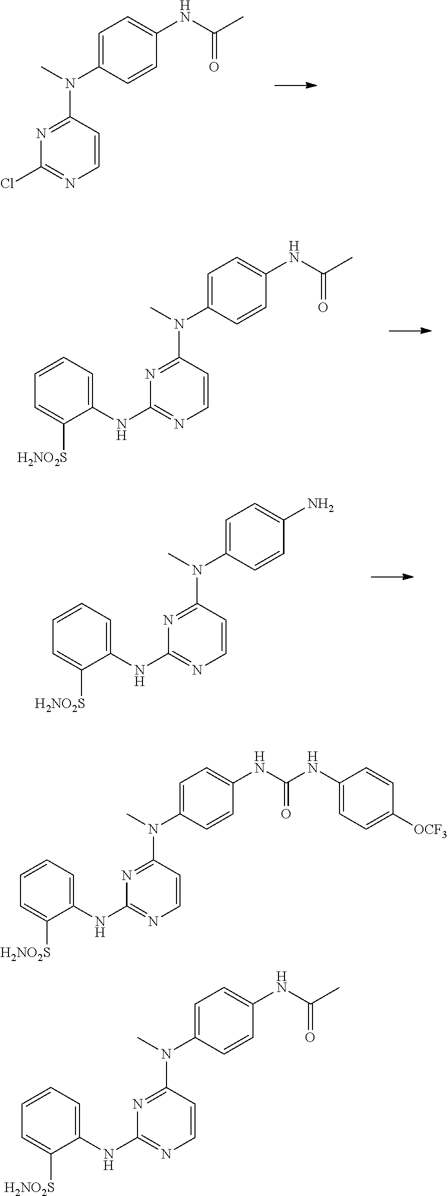 [SMILES: CC(=O)Nc1ccc(N(C)c2ccnc(Cl)n2)cc1 ; CC(=O)Nc1ccc(N(C)c2ccnc(Nc3ccccc3S(N)(=O)=O)n2)cc1 ; CN(c1ccc(N)cc1)c1ccnc(Nc2ccccc2S(N)(=O)=O)n1 ; CN(c1ccc(NC(=O)Nc2ccc(OC(F)(F)F)cc2)cc1)c1ccnc(Nc2ccccc2S(N)(=O)=O)n1 ; CC(=O)Nc1ccc(N(C)c2ccnc(Nc3ccccc3S(N)(=O)=O)n2)cc1]

### Step 1: N-(4-(Methyl(2-(2-sulfamoylphenylamino)pyrimidin-4-yl)amino)phenyl)-acetamide

N-(4-((2-chloropyrimidin-4-yl)(methyl)amino)phenyl)acetamide (1.15 g, 4.16 mmol) and 2-aminobenzenesulfonamide (716 mg, 4.16 mmol) were dissolved in i-PrOH (40 mL). Concentrated HCl (10 drops) was added dropwise to the stirred solution. The reaction mixture was stirred for 16 hours at 80° C. and cooled to room temperature. The pale-pink precipitate formed was collected by filtration, washed with EtOAc and dried under vacuum to afford the titled compound as a white solid (1.48 g, 86%). ¹H NMR (600 MHz, DMSO-d₆): δ ppm 2.05 (s, 3H), 3.35 (s, 3H), 7.28 (d, J=8.7 Hz, 2H), 7.30-7.45 (m, 3H), 7.50-7.65 (m, 2H), 7.70 (d, J=8.7 Hz, 2H), 7.92 (m, 1H), 10.28 (br s, 1H). LCMS (Method A): 3.80 min [MH]⁺=413.4.

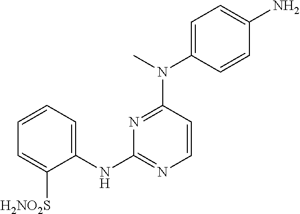 [SMILES: CN(c1ccc(N)cc1)c1ccnc(Nc2ccccc2S(N)(=O)=O)n1]

### Step 2: 2-(4-((4-aminophenyl)(methyl)amino)pyrimidin-2-ylamino)benzene-sulfonamide

N-(4-(methyl(2-(2-sulfamoylphenylamino)pyrimidin-4-yl)amino)phenyl)acetamide (1.47 g, 3.56 mmol) was dissolved in MeOH (40 mL). Acetyl chloride (5.07 mL, 71.3 mmol) was added dropwise and the reaction mixture was stirred at room temperature for 48 hours and concentrated in vacuo to afford the titled compound (1.21 g, 92%) as a white solid.

¹H NMR (600 MHz, MeOD-d₄): δ ppm 3.51 (s, 3H), 6.00 (br s, 1H), 7.48-8.06 (m, 14H).

LCMS (Method A): 1.08 min [MH]⁺=371.1.

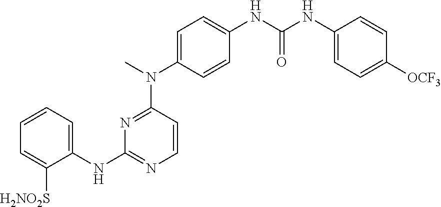 [SMILES: CN(c1ccc(NC(=O)Nc2ccc(OC(F)(F)F)cc2)cc1)c1ccnc(Nc2ccccc2S(N)(=O)=O)n1]

### Step 3: 2-(4-(Methyl(4-(3-(4-(trifluoromethoxy)phenyl)ureido)phenyl)amino)pyrimidin-2-ylamino)benzenesulfonamide

Following general procedure A using 2-(4-((4-aminophenyl)(methyl)amino)pyrimidin-2-ylamino)benzenesulfonamide (100 mg, 0.27 mmol) and (4-trifluoromethoxyphenyl)isocyanate (0.27 mmol), the titled compound (28 mg, 21%) was obtained as a white solid. ¹H NMR (600 MHz, MeOD-d₄): δ ppm 3.46 (s, 3H), 6.01 (br s, 1H), 6.77 (d, J=9.0 Hz, 1H), 7.00 (d, J=9.0 Hz, 1H), 7.20 (d, J=8.4 Hz, 2H), 7.25 (d, J=9.0 Hz, 2H), 7.27 (m, 1H), 7.53 (d, J=9.0 Hz, 2H), 7.59 (d, J=8.4 Hz, 2H), 7.79 (m, 1H), 7.92-7.97 (m, 2H). LCMS (Method A): 4.71 min [MH]⁺=574.4.

Compound 6

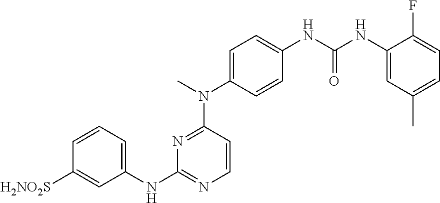 [SMILES: Cc1ccc(F)c(NC(=O)Nc2ccc(N(C)c3ccnc(Nc4cccc(S(N)(=O)=O)c4)n3)cc2)c1]

### 3-(3-((4-(3-(2-fluoro-5 methylphenyl)ureido)phenyl)(methyl)amino)phenylamino)benzenesulfonamide

Following general procedure A using intermediate A (100 mg, 0.27 mmol) and 2-fluoro-5-methylphenylisocyanate (41 mg, 0.27 mmol), 3-(3-((4-(3-(2-fluoro-5 methylphenyl)ureido)phenyl)(methyl)amino)phenylamino)benzenesulfonamide (9.8 mg, 7%) was obtained as a white solid. ¹H NMR (600 MHz, MeOD-d₄): δ ppm 3.52 (s, 3H), 5.88 (d, J=5.4 Hz, 1H), 6.82 (m, 1H), 6.99 (m, 1H), 7.24 (d, J=9.0 Hz, 2H), 7.42 (m, 1H), 7.52 (d, J=7.8 Hz, 1H), 7.60 (d, J=9.0 Hz, 2H), 7.66 (d, J=10.2 Hz, 1H), 7.76 (d, J=7.8 Hz, 1H), 7.90 (8.17 (m, 1H), 8.17 (s, 1H), 8.57 (br s, 1H). LCMS (Method A): 4.37 min [MH]⁺=522.2.

Compound 7

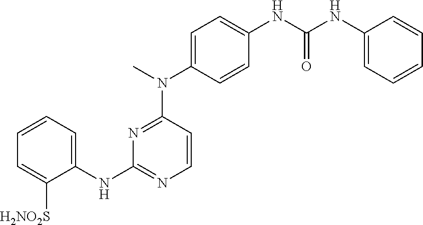 [SMILES: CN(c1ccc(NC(=O)Nc2ccccc2)cc1)c1ccnc(Nc2ccccc2S(N)(=O)=O)n1]

### 2-(4-(Methyl(4-(3-(4-(trifluoromethoxy)phenyl)ureido)phenyl)amino)pyrimidin-2-ylamino)benzenesulfonamide

Following general procedure A using 2-(4-((4-aminophenyl)(methyl)-amino)pyrimidin-2-ylamino)benzenesulfonamide (100 mg, 0.27 mmol) and phenylisocyanate (32 mg, 0.27 mmol), the titled compound (3.5 mg, 3%) was obtained as a white solid. ¹H NMR (600 MHz, MeOD-d₄): δ ppm 3.46 (s, 3H), 6.01 (br s, 1H), 6.77 (d, J=9.0 Hz, 1H), 7.00 (d, J=9.0 Hz, 1H), 7.20 (d, J=8.4 Hz, 2H), 7.25 (d, J=9.0 Hz, 2H), 7.27 (m, 1H), 7.53 (d, J=9.0 Hz, 2H), 7.59 (d, J=8.4 Hz, 2H), 7.79 (m, 1H), 7.92-7.97 (m, 2H). LCMS (acidic 10 min): 4.71 min [MH]⁺=574.4.

Compound 8

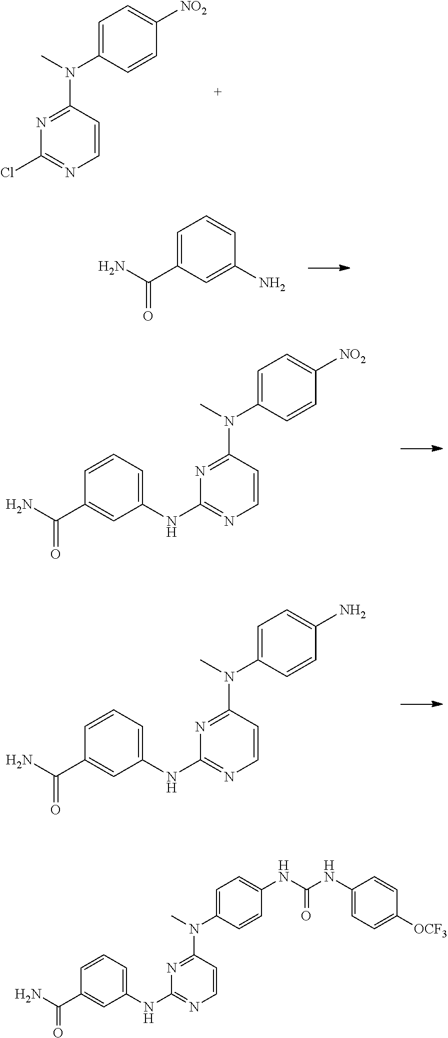 [SMILES: CN(c1ccc([N+](=O)[O-])cc1)c1ccnc(Cl)n1 ; NC(=O)c1cccc(N)c1 ; CN(c1ccc([N+](=O)[O-])cc1)c1ccnc(Nc2cccc(C(N)=O)c2)n1 ; CN(c1ccc(N)cc1)c1ccnc(Nc2cccc(C(N)=O)c2)n1 ; CN(c1ccc(NC(=O)Nc2ccc(OC(F)(F)F)cc2)cc1)c1ccnc(Nc2cccc(C(N)=O)c2)n1]

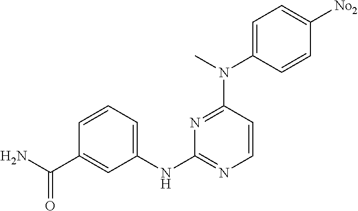

### Step 1: 3-(4-(N-Methyl-N-(4-nitrophenyl)amino)pyrimidin-2-ylamino)benzamide

2-Chloro-N-methyl-N-(4-nitrophenyl)pyrimidin-4-amine (from step 2 of preparation of intermediate A, 200 mg, 0.77 mmol) and 3-aminobenzamide (110 mg, 0.80 mmol) were dissolved in t-BuOH (5 mL) and conc HCl (2 drops) was added. The reaction mixture was heated to 160° C. for 2 hours. The solvent was removed and the crude was rinsed with petroleum ether:ethyl acetate (2:1) to give 3-(4-(N-Methyl-N-(4-nitrophenyl)amino)pyrimidin-2-ylamino)benzamide (180 mg, 64%) as a yellow solid which was used in next step without further purification. LCMS (Method B): 0.67 min [MH]⁺=365.2.

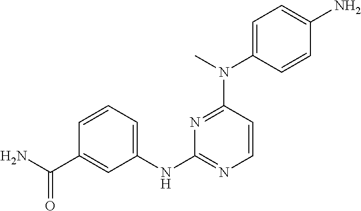 [SMILES: CN(c1ccc(N)cc1)c1ccnc(Nc2cccc(C(N)=O)c2)n1]

### Step 2: 3-(4-(N-methyl-N-(4-amidephenyl)amino) pyrimidin-2-ylamino) benzamide

3-(4-(N-Methyl-N-(4-nitrophenyl)amino)pyrimidin-2-ylamino)benzamide (180 mg, 0.5 mmol) was dissolved in ethanol (20 mL) and 10% wet Pd/C (20 mg) was added to the solution. The reaction mixture was stirred under hydrogen overnight. The catalyst was removed by filtration and the solvent was removed under reduced pressure to give 3-((4-((4-aminophenyl)(methyl)amino)pyrimidin-2-yl)amino)benzamide (150 mg, 88%) as a yellow solid which was used in next step without further purification. LCMS (Method B): 0.36 min [MH]⁺=335.1.

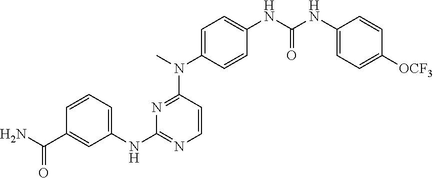 [SMILES: CN(c1ccc(NC(=O)Nc2ccc(OC(F)(F)F)cc2)cc1)c1ccnc(Nc2cccc(C(N)=O)c2)n1]

### Step 3: 3-((4-(methyl(4-(3-(4-(trifluoromethoxy)phenyl)ureido)phenyl)amino) pyrimidin-2-yl)amino) benzamide

To a solution of 3-(4-(N-methyl-N-(4-amidephenyl)amino)pyrimidin-2-ylamino) benzamide (150 mg, 0.45 mmol) in THF (20 mL) were added 1-isocyanato-4-(trifluoromethoxy)benzene (95 mg, 0.47 mmol) and DIEA (116 mg, 0.9 mmol). The reaction was stirred at room temperature overnight. The mixture was concentrated in vacuo and the crude was purified by column chromatography with eluent (DCM:methanol, 100 to 100:2) to give 3-((4-(methyl(4-(3-(4-(trifluoromethoxy) phenyl)ureido)phenyl)amino)pyrimidin-2-yl)amino) benzamide (152 mg, 63%) as a yellow solid. ¹H NMR (400 MHz, DMSO-d₆): δ ppm 3.43 (s, 3H), 5.79 (d, J=6.0 Hz, 1H), 7.38 (m, 7H), 7.59 (m, 4H), 7.88 (m, 3H), 8.33 (s, 1H), 8.99 (m, 2H), 9.28 (s, 1H), 9.47 (s, 1H). LCMS (Method B): 2.47 min [MH]⁺=538.3.

Compound 9

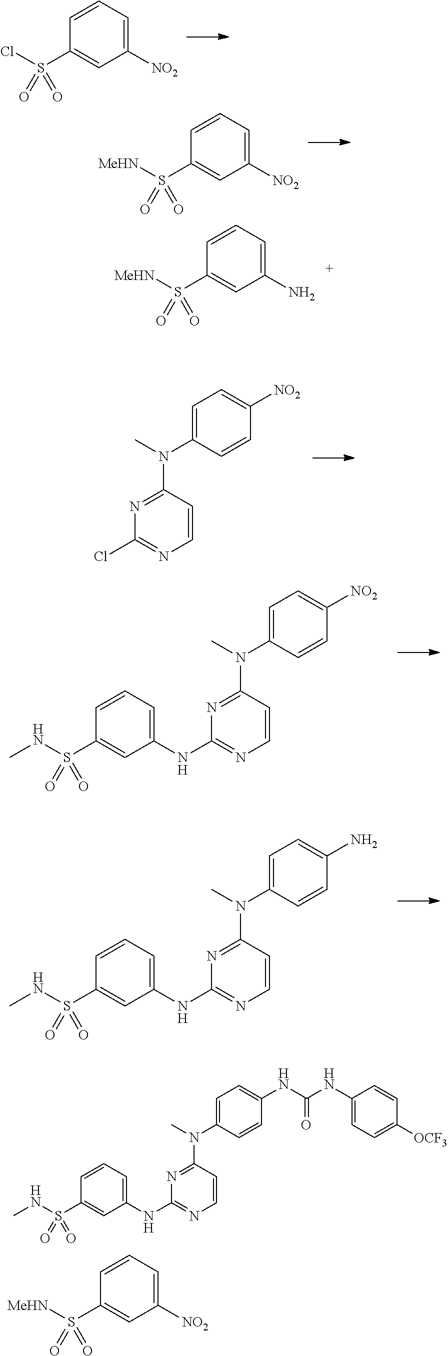

### Step 1: N-methyl-3-nitrobenzenesulfonamide

To a solution of 3-nitrobenzene-1-sulfonyl chloride (1.0 g, 4.52 mmol) in THF (15 mL) were added methylamine hydrochloride (366 mg, 5.42 mmol) and N,N-diisopropylethylamine (1.75 g, 13.6 mmol). The mixture was stirred at room temperature overnight. The reaction mixture was poured into water (20 mL). The precipitate formed was collected via filtering. The solid was dried on a rotary evaporator to give N-methyl-3-nitrobenzenesulfonamide (800 mg, 82%) as a white solid. LCMS (Method B): 0.75 min [MH]⁼217.1.

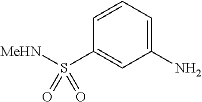 [SMILES: CNS(=O)(=O)c1cccc(N)c1]

### Step 2: 3-amino-N-methylbenzenesulfonamide

To a solution of N-methyl-3-nitrobenzenesulfonamide (800 mg, 3.70 mmol) in methanol (20 mL) was added palladium on carbon (10%, 80 mg). The mixture was stirred at room temperature overnight under hydrogen atmosphere. The reaction mixture was filtered to remove the solid and the filtrate was concentrated to dryness under reduced pressure to give 3-amino-N-methylbenzenesulfonamide (630 mg, 92%) as a light brown solid.

LCMS (Method B): 0.33 min [MH]⁺=187.2.

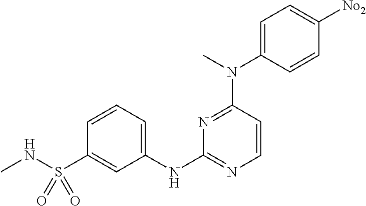

### Step 3: N-methyl-3-((4-(methyl(4-nitrophenyl)amino)pyrimidin-2-yl)amino)benzene sulfonamide

To a solution of 3-amino-N-methylbenzenesulfonamide (180 mg, 0.97 mmol) in tert-butanol (5 mL) were added 2-chloro-N-methyl-N-(4-nitrophenyl)pyrimidin-4-amine (from step 2 of the preparation of Compound 1, 244 mg, 0.92 mmol) and concentrated HCl aqueous solution (2 drops). The resulting mixture was stirred at 160° C. for 5 h. TLC and LCMS analyses indicated that the reaction was complete. The mixture was allowed to cool down to room temperature. The solid was collected via filtration and dried under reduced pressure to give N-methyl-3-((4-(methyl(4-nitrophenyl)amino)pyrimidin-2-yl)amino)benzenesulfonamide (300 mg, 79%) as a light yellow sold. LCMS (Method B): 2.15 min [MH]⁺=415.1.

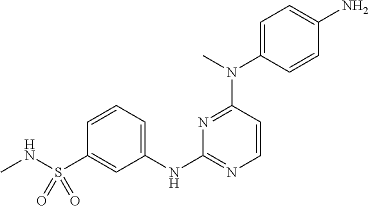 [SMILES: CNS(=O)(=O)c1cccc(Nc2nccc(N(C)c3ccc(N)cc3)n2)c1]

### Step 4: N-Methyl-3-((4-(methyl(4-(3-(4-(trifluoromethoxy)phenyl)ureido)-phenyl)amino) pyrimidin-2-yl)amino) benzenesulfonamide

To a solution of N-methyl-3-((4-(methyl (4-nitrophenyl)amino) pyrimidin-2-yl)amino) benzenesulfonamide (300 mg, 0.72 mmol) in methanol (25 mL) were added zinc powder (1.0 g) and saturated ammonium chloride aqueous solution (25 mL). The resulting mixture was stirred at room temperature overnight. The solid was filtered off and the solvent was removed under reduced pressure to give a residue which was partitioned between water and ethyl acetate. The organic phase was separated and washed with water, brine, dried over sodium sulfate and concentrated to dryness to give 3-((4-((4-aminophenyl)(methyl)amino)pyrimidin-2-yl)amino)-N-methylbenzene-sulfonamide (250 mg, 90%) as a dark brown solid. LCMS (Method B): 1.27 min [MH]⁺=385.2.

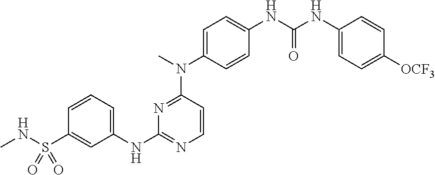 [SMILES: CNS(=O)(=O)c1cccc(Nc2nccc(N(C)c3ccc(NC(=O)Nc4ccc(OC(F)(F)F)cc4)cc3)n2)c1]

### Step 5: N-methyl-3-((4-(methyl(4-(3-(4-(trifluoromethoxy)phenyl)ureido)-phenyl)amino) pyrimidin-2-yl)amino) benzenesulfonamide

N-Methyl-3-((4-(methyl(4-(3-(4-(trifluoromethoxy)phenyl)ureido)phenyl)amino)-pyrimidin-2-yl)amino)benzenesulfonamide. To a solution of 3-((4-((4-aminophenyl)(methyl)amino) pyrimidin-2-yl)amino)-N-methylbenzenesulfonamide (250 mg, 0.65 mmol) in tetrahydrofuran (25 mL) were added 4-(trifluoromethoxy)phenyl isocyanate (158 mg, 0.78 mmol) and N,N-diisopropylethylamine (168 mg, 1.30 mmol). The mixture was stirred at room temperature overnight. The reaction mixture was concentrated to dryness under reduced pressure to give a residue which was purified by silica-gel chromatography (dichloromethane,/methanol, 30:1) to give N-methyl-3-((4-(methyl(4-(3-(4-(trifluoromethoxy)phenyl)ureido)phenyl)amino)pyrimidin-2-yl)amino)benzene sulfonamide (210 mg, 55%) as a white solid. ¹H NMR (400 MHz, DMSO-d₆): δ ppm 2.45 (d, J=5.0 Hz, 3H), 3.50 (s, 3H), 5.95 (br s, 1H), 7.32 (t, J=8.5 Hz, 4H), 7.49-7.45 (m, 2H), 7.68-7.54 (m, 5H), 7.77 (dd, J=8.0 and 1.2 Hz, 1H), 7.89 (d, J=6.8 Hz, 1H), 8.39 (s, 1H), 9.19 (s, 2H), 10.49 (s, 1H). LCMS (Method B): 2.57 min [MH]⁺=588.2.

Compound 10

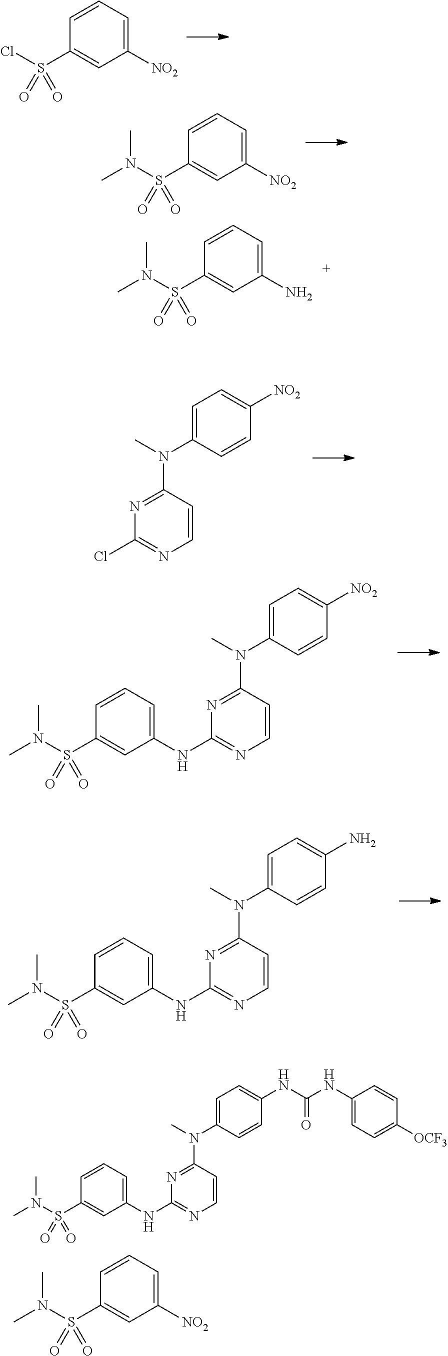

### Step 1: N,N-Dimethyl-3-nitrobenzenesulfonamide

In a bottom flask, Me₂NH₂.HCl (442 mg, 5.42 mmol) and DIEA (1.75 g, 13.6 mmol) were added in THF (15 mL) and the solution was cooled to 0° C. 3-nitrobenzene-1-sulfonyl chloride (1 g, 4.52 mmol) was added slowly and the reaction mixture was stirred at room temperature. The mixture was poured into water (50 mL), extracted with EtOAc (3×30 mL). The combined organic layers were washed with water (3×30 mL), dried over Na₂SO₄. The solvent was removed under reduced pressure to give N,N-dimethyl-3-nitrobenzenesulfonamide (850 mg, 83%) as a yellow solid which was used in next step directly. LCMS (Method B): 1.31 [M+H]⁺=231.1

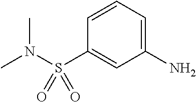

### Step 2: 3-amino-N,N-dimethylbenzenesulfonamide

N,N-Dimethyl-3-nitrobenzenesulfonamide (850 mg, 3.7 mmol) was dissolved in methanol (20 mL). The wet Pd/C (85 mg) was added to the mixture. The reaction mixture was stirred under hydrogen atmosphere overnight. The catalyst was removed by filtration and the solvent was removed under reduced pressure to give 3-amino-N,N-dimethylbenzenesulfonamide (600 mg, 80%) as a white solid. LCMS (Method B): 0.48 [M+H]⁺=201.1

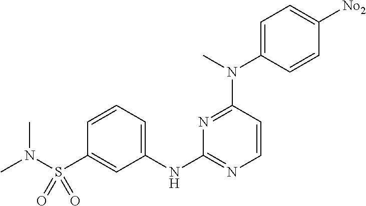

### Step 3: N,N-dimethyl-3-((4-(methyl(4-nitrophenyl)amino)pyrimidin-2-yl)amino)benzene sulfonamide

2-Chloro-N-methyl-N-(4-nitrophenyl)pyrimidin-4-amine (from step 2 of the preparation of Compound 1, 200 mg, 0.77 mmol) and 3-amino-N,N-dimethylbenzenesulfonamide (158 mg, 0.79 mmol) were dissolved in t-BuOH (5 mL), followed by addition of concentrated HCl (2 drops). The reaction mixture was heated to 160° C. for 2 hours. The solvent was removed and the crude was purified by re-crystallization from petroleum ether/ethylacetate, 1:1, to give the desired compound (220 mg, 68%) as a yellow solid. LCMS (Method B): 2.16 min [MH]⁺=429.2.

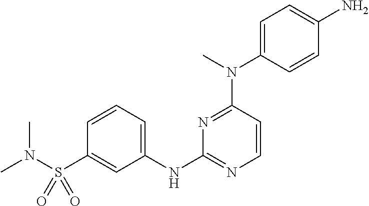 [SMILES: CN(c1ccc(N)cc1)c1ccnc(Nc2cccc(S(=O)(=O)N(C)C)c2)n1]

### Step 4: 3-((4-((4-aminophenyl)(methyl)amino)pyrimidin-2-yl)amino)-N,N-dimethyl benzene sulfonamide

N,N-Dimethyl-3-((4-(methyl(4-nitrophenyl)amino)pyrimidin-2-yl)amino) benzenesulfonamide (220 mg, 0.51 mmol) was dissolved in ethanol (30 mL), followed by addition of wet Pd/C (10%, 22 mg). The reaction mixture was stirred under hydrogen overnight. The catalyst was removed by filtration and the filtrate was concentrated under reduced pressure to give 3-((4-((4-aminophenyl)(methyl)amino)pyrimidin-2-yl)amino)-N,N-dimethylbenzene sulfonamide (150 mg, 75%) as a yellow solid which was used in next step directly. LCMS (Method B): 1.87 min [MH]⁺=399.2.

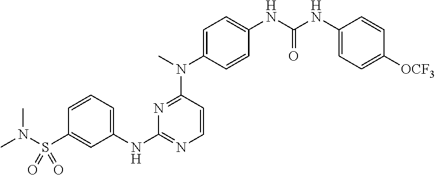 [SMILES: CN(c1ccc(NC(=O)Nc2ccc(OC(F)(F)F)cc2)cc1)c1ccnc(Nc2cccc(S(=O)(=O)N(C)C)c2)n1]

### Step 5: N,N-dimethyl-3-((4-(methyl(4-(3-(4-(trifluoromethoxy)phenyl)ureido)-phenyl)amino)pyrimidin-2-yl)amino)benzenesulfonamide

N,N-Dimethyl-3-((4-(methyl(4-(3-(4-(trifluoromethoxy)phenyl)ureido)phenyl)-amino) pyrimidin-2-yl)amino)benzenesulfonamide. To a solution of 3-((4-((4-aminophenyl)(methyl)amino)pyrimidin-2-yl)amino)-N,N-dimethylbenzene sulfonamide (150 mg, 0.38 mmol) in THF (20 mL) were added 1-isocyanato-4-(trifluoromethoxy)benzene (80 mg, 0.4 mmol) and DIEA (98 mg, 0.76 mmol). The reaction was stirred at rt overnight. The mixture was concentrated in vacuo and the crude was purified by column chromatography with eluent (DCM/methanol, 100 to 100/2) to give the impure product which was re-crystallized from petroleum ether/ethanol, 1:1 to give N,N-dimethyl-3-((4-(methyl(4-(3-(4-(trifluoromethoxy) phenyl)ureido)phenyl)amino)pyrimidin-2-yl)amino)benzenesulfonamide (42.6 mg, 19%) as a white solid. ¹H NMR (400 MHz, DMSO-d₆): δ ppm 2.63 (s, 6H) 3.45 (s, 3H), 5.81 (d, J=6.0 Hz, 1H), 7.32 (m, 5H), 7.50 (t, J=8.0 Hz, 1H), 7.59 (m, 4H), 7.93 (m, 2H), 8.53 (s, 1H), 8.89 (s, 1H), 8.94 (s, 1H), 9.60 (s, 1H). LCMS (Method B): 2.66 min [MH]⁺=602.3.

Compound 11

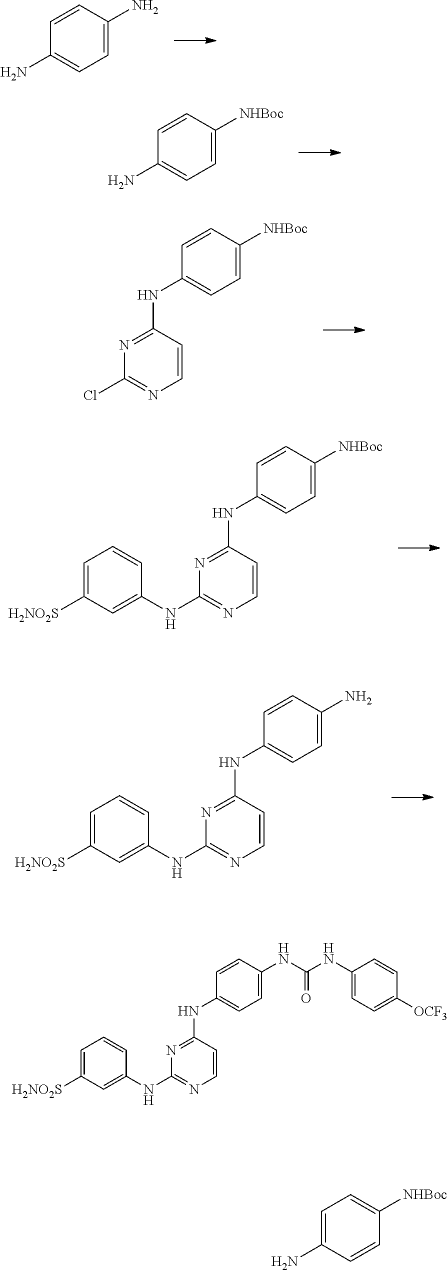

### Step 1: tert-butyl (4-aminophenyl)carbamate

Benzene-1,4-diamine (3.24 g, 30 mmol) was dissolved in THF (30 mL), DMF (10 mL) and water (5 mL), followed by addition of potassium carbonate (1.52 g, 11 mmol), and Boc₂0 (2.18 g, 10 mmol). The reaction mixture was stirred at room temperature overnight. The mixture was poured into water (50 mL), and the resulting mixture was extracted three times with ethylacetate (50 mL). The combined organic layers were washed three times with water (50 mL), dried over Na₂SO₄ to give tert-butyl (4-aminophenyl)carbamate (1.6 g, 77%) as a yellow solid. LCMS (Method B): 0.51 min [MH]⁺=209.1.

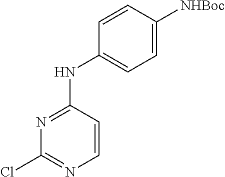 [SMILES: CC(C)(C)OC(=O)Nc1ccc(Nc2ccnc(Cl)n2)cc1]

### Step 2: tert-butyl (4-((2-chloropyrimidin-4-yl)amino)phenyl)carbamate

Tert-butyl 4-aminophenylcarbamate (800 mg, 3.84 mmol), 2,4-dichloropyrimidine (744 mg, 5.00 mmol) and NaHCO₃ (967 mg, 11.5 mmol) were dissolved in 2-propanol (20 mL). The reaction mixture was heated to 90° C. overnight. The hot reaction mixture was filtered to remove the solid. The filtrate was concentrated to give a residue. To the residue was added DCM (80 mL) and the suspension was stirred for 30 min. The precipitated solid was collected by filtration to give tert-butyl (4-((2-chloropyrimidin-4-yl)amino)phenyl)carbamate (1 g, 63%) as a white solid. LCMS (Method B): 2.63 min [MH]⁺=321.1.

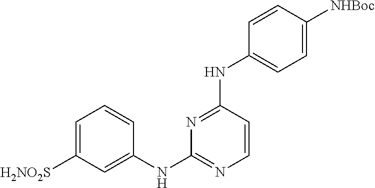 [SMILES: CC(C)(C)OC(=O)Nc1ccc(Nc2ccnc(Nc3cccc(S(N)(=O)=O)c3)n2)cc1]

### Step 3: tert-butyl (4-((2-((3-sulfamoylphenyl)amino)pyrimidin-4-yl)amino)phenyl) carbamate

Tert-butyl 4-(2-chloropyrimidin-4-ylamino)phenylcarbamate (200 mg, 0.62 mmol) and 3-amino-benzenesulfonamide (107 mg, 0.62 mmol) were dissolved in 1,4-dioxane (4 mL), followed by addition of TsOH (95 mg, 0.5 mmol). The reaction mixture was heated to 120° C. under microwave for 2 hours. TLC analysis showed the reaction was complete. The solvent was removed and the crude product was washed with petroleum ether:EtOAc (2:1) and dried to give tert-butyl (4-((2-((3-sulfamoylphenyl)amino)pyrimidin-4-yl)amino)phenyl)-carbamate (120 mg, 42.3%) as a yellow solid.

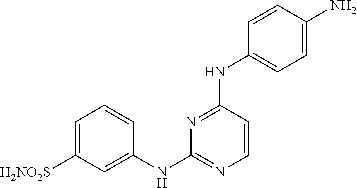 [SMILES: Nc1ccc(Nc2ccnc(Nc3cccc(S(N)(=O)=O)c3)n2)cc1]

### Step 4: 3-((4-((4-aminophenyl)amino)pyrimidin-2-yl)amino)benzenesulfonamide

Tert-butyl(4-((2-((3-sulfamoylphenyl)amino)pyrimidin-4-yl)amino)phenyl)carbamate (120 mg, 0.26 mmol) was dissolved in DCM (10 mL), followed by addition of TFA (1 mL). The reaction mixture was stirred at room temperature overnight. The solvent was removed to give the crude which was washed with aqueous NaHCO₃, dried to give 3-((4-((4-aminophenyl)amino)pyrimidin-2-yl)amino)benzenesulfonamide (80 mg, 86.3%) as a yellow solid. LCMS (Method B): 0.29 min [MH]⁺=357.1.

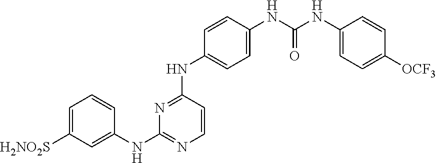 [SMILES: NS(=O)(=O)c1cccc(Nc2nccc(Nc3ccc(NC(=O)Nc4ccc(OC(F)(F)F)cc4)cc3)n2)c1]

### Step 5: 3-((4-((4-(3-(4-(trifluoromethoxy)phenyl)ureido)phenyl)amino)pyrimidin-2-yl)amino)benzenesulfonamide

3-((4-((4-(3-(4-(Trifluoromethoxy)phenyl)ureido)phenyl)amino)pyrimidin-2-yl)amino) benzenesulfonamide. To a solution of 3-((4-((4-aminophenyl)amino)pyrimidin-2-yl)amino)benzenesulfonamide (80 mg, 0.22 mmol) in THF (20 mL) was added 1-isocyanato-4-(trifluoromethoxy)benzene (48 mg, 0.24 mmol) and DIEA (57 mg, 0.44 mmol). The reaction was stirred at room temperature overnight. The mixture was concentrated in vacuo and the crude was purified by column chromatography (DCM:methanol, 100:0 to 100:5) to give 3-((4-((4-(3-(4-(trifluoromethoxy)phenyl) ureido)phenyl)amino)pyrimidin-2-yl)amino)benzenesulfonamide (17.8 mg, 14.5%) as a yellow solid. ¹H NMR (400 MHz, DMSO-d₆): δ ppm 6.38 (d, J=6.8 Hz, 1H), 7.58 (m, 13H), 7.98 (m, 3H), 8.84 (s, 1H), 8.98 (s, 1H), 10.24 (s, 1H). LCMS (Method B): 2.46 min [MH]⁺=560.1 [MNa]⁺=582.1

Compound 12

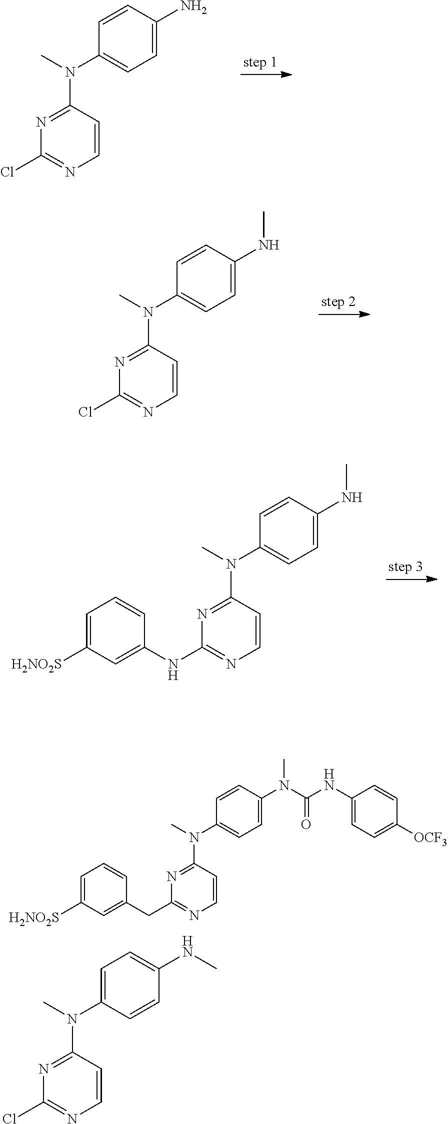

### Step 1: N1-(2-chloropyrimidin-4-yl)-N1,N4-dimethylbenzene-1,4-diamine

N1-(2-Chloropyrimidin-4-yl)-N1-methylbenzene-1,4-diamine (from step 2 of the preparation intermediate C, 700 mg, 2.98 mmol), Paraformaldehyde (98.4 mg, 3.28 mmol) were dissolved in 1,2-dichloroethane (16 mL) and methanol (8 mL), followed by addition of acetic acid (3 drops). The reaction mixture was heated to 40° C. overnight, and then NaBH₃CN (281 mg, 4.47 mmol) was added. The solvent was removed and the crude was purified by column chromatography (petroleum ether: ethylacetate, 3:1) to give N1-(2-chloropyrimidin-4-yl)-N1,N4-dimethylbenzene-1,4-diamine (250 mg, 30%) as a white solid. LCMS (Method B): 2.22 min [MH]⁺=249.1.

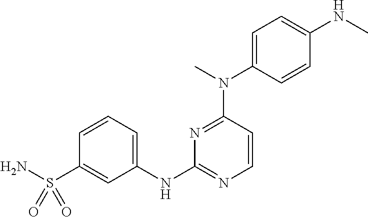 [SMILES: CNc1ccc(N(C)c2ccnc(Nc3cccc(S(N)(=O)=O)c3)n2)cc1]

### Step 2: 3-((4-(methyl(4-(methylamino)phenyl)amino)pyrimidin-2-yl)amino)benzene sulfonamide

N1-(2-Chloropyrimidin-4-yl)-N1,N4-dimethylbenzene-1,4-diamine (50 mg, 0.20 mmol), 3-amino-benzenesulfonamide (35 mg, 0.20 mmol) were dissolved in 1,4-dioxane (3 mL), followed by addition of TsOH (30 mg, 0.16 mmol). The reaction mixture was heated to 120° C. under microwave for 2 hours. The solvent was removed and the crude was purified by column chromatography (DCM/methanol, 100:0 to 100:2) then by prep HPLC to give 3-((4-(methyl(4-(methylamino)phenyl)amino)pyrimidin-2-yl)amino) benzenesulfonamide (16 mg, 21%) as a yellow solid. ¹H NMR (400 MHz, DMSO-d₆): δ ppm 2.72 (s, 3H), 3.48 (s, 3H), 5.89 (s, 1H), 6.67 (d, J=8.0 Hz, 2H), 7.13 (m, 2H), 7.73 (m, 6H), 7.85 (d, J=6.8 Hz, 1H), 8.37 (s, 1H), 10.73 (s, 1H). LCMS (Method B): 0.86 min [MH]⁺=385.1

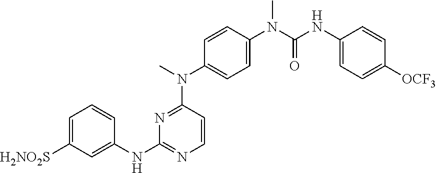 [SMILES: CN(C(=O)Nc1ccc(OC(F)(F)F)cc1)c1ccc(N(C)c2ccnc(Nc3cccc(S(N)(=O)=O)c3)n2)cc1]

### Step 3: 3-((4-(methyl(4-(1-methyl-3-(4-(trifluoromethoxy)phenyl)ureido)phenyl)amino)pyrimidin-2-yl)amino) benzene sulfonamide

3-((4-(Methyl(4-(1-methyl-3-(4-(trifluoromethoxy)phenyl)ureido)phenyl)amino) pyrimidin-2-yl)amino)benzenesulfonamide. To a solution of 3-((4-(methyl(4-(methylamino)phenyl)amino)pyrimidin-2-yl)amino)benzenesulfonamide (from step 2 above, 150 mg, 0.39 mmol) in THF (10 mL) was added 1-isocyanato-4-(trifluoromethoxy)benzene (87 mg, 0.43 mmol) and DIEA (101 mg, 0.78 mmol). The reaction was stirred at room temperature overnight. The mixture was concentrated in vacuo and the crude was purified by column chromatography (DCM:methanol, 100 to 100:5), to give a yellow solid which was purified by preparative HPLC to give 3-((4-(methyl(4-(1-methyl-3-(4-(trifluoromethoxy)phenyl)ureido)phenyl)amino)pyrimidin-2-yl)amino)benzene-sulfonamide (40 mg, 17%) as a white solid. ¹H NMR (400 MHz, DMSO-d₆): δ ppm 3.34 (s, 3H), 3.53 (s, 3H), 6.09 (d, J=6.4 Hz, 1H), 7.28 (d, J=8.8 Hz, 2H), 7.45 (m, 4H), 7.59 (m, 6H), 7.75 (m, 1H), 7.96 (d, J=7.2 Hz, 1H), 8.39 (s, 1H), 8.58 (s, 1H), 10.51 (s, 1H). LCMS (Method B): 2.40 min [MH]⁺=588.2 [MNa]⁺=610.1.

Compound 13

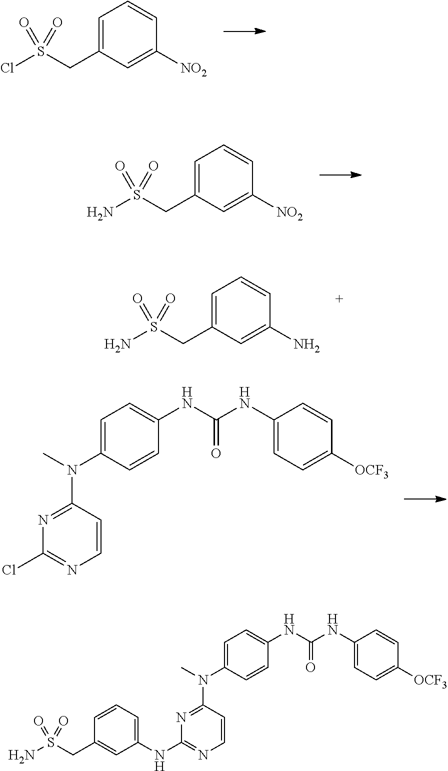 [SMILES: O=[N+]([O-])c1cccc(CS(=O)(=O)Cl)c1 ; NS(=O)(=O)Cc1cccc([N+](=O)[O-])c1 ; Nc1cccc(CS(N)(=O)=O)c1 ; CN(c1ccc(NC(=O)Nc2ccc(OC(F)(F)F)cc2)cc1)c1ccnc(Cl)n1 ; CN(c1ccc(NC(=O)Nc2ccc(OC(F)(F)F)cc2)cc1)c1ccnc(Nc2cccc(CS(N)(=O)=O)c2)n1]

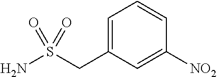 [SMILES: NS(=O)(=O)Cc1cccc([N+](=O)[O-])c1]

### Step 1: (3-nitrophenyl)methanesulfonamide

To a mixture of (3-nitrophenyl)methanesulfonyl chloride (700 mg, 2.97 mmol) in acetonitrile (3 mL) was added concentrated ammonia saturated with ammonium carbonate. The resultant mixture was stirred at rt for 2 h. The mixture was concentrated and the residue was diluted with cold water leading to the formation of precipitate which was filtered off and washed with water to give (3-nitrophenyl)methanesulfonamide (642 mg, 100%) as a white solid. LCMS (Method B): 0.47 min [MNa]⁺=239.0.

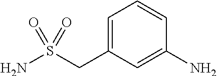 [SMILES: Nc1cccc(CS(N)(=O)=O)c1]

### Step 2: (3-aminophenyl)methanesulfonamide

To a mixture of (3-nitrophenyl)methanesulfonamide (150 mg, 0.69 mmol) in methanol (4 mL) and NH₄Cl solution (4 mL) was added zinc (453 mg, 6.9 mmol). The mixture was stirred at 65° C. for 3 h. The mixture was adjusted to pH=7 with sodium hydrogen carbonate solution. The solid was filtered off. The filtrate was extracted with ethylacetate (3×). The combined organic layers were dried over sodium sulfate and concentrated to give (3-aminophenyl)methanesulfonamide (100 mg, 78%) as a yellow oil. LCMS (Method B): 0.28 min [MH]⁺=187.0.

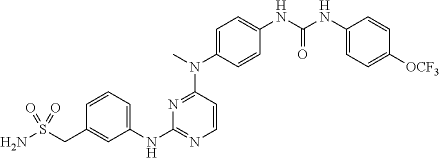 [SMILES: CN(c1ccc(NC(=O)Nc2ccc(OC(F)(F)F)cc2)cc1)c1ccnc(Nc2cccc(CS(N)(=O)=O)c2)n1]

### Step 3: (3-((4-(Methyl(4-(3-(4-(trifluoromethoxy)phenyl)ureido)phenyl)amino) pyrimidin-2-yl)amino) phenyl)methanesulfonamide

Following general procedure C using intermediate C (117 mg, 0.27 mmol) and 3-aminobenzenemethane sulfonamide (50 mg, 0.27 mmol), (3-((4-(Methyl(4-(3-(4-(trifluoromethoxy)phenyl)ureido)phenyl)amino)pyrimidin-2-yl)amino)phenyl) methanesulfonamide (100 mg, 45%) was obtained as a white solid. ¹H NMR (400 MHz, MeOD-d₄): δ ppm 3.59 (s, 3H), 4.41 (s, 2H), 5.97 (d, J=7.4 Hz, 1H), 7.36-7.15 (m, 6H), 7.47 (d, J=7.9 Hz, 1H), 7.57 (m, 2H), 7.72-7.61 (m, 3H), 7.84 (s, 1H). LCMS (Method B): 2.48 min [MH]⁺=588.2.

Compound 14

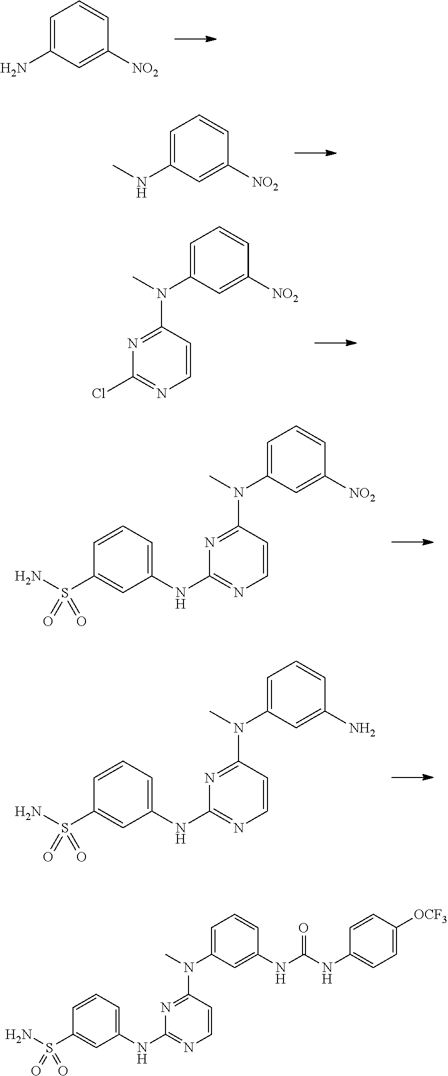 [SMILES: Nc1cccc([N+](=O)[O-])c1 ; CNc1cccc([N+](=O)[O-])c1 ; CN(c1cccc([N+](=O)[O-])c1)c1ccnc(Cl)n1 ; CN(c1cccc([N+](=O)[O-])c1)c1ccnc(Nc2cccc(S(N)(=O)=O)c2)n1 ; CN(c1cccc(N)c1)c1ccnc(Nc2cccc(S(N)(=O)=O)c2)n1 ; CN(c1cccc(NC(=O)Nc2ccc(OC(F)(F)F)cc2)c1)c1ccnc(Nc2cccc(S(N)(=O)=O)c2)n1]

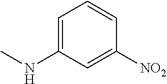

### Step 1: N-methyl-3-nitroaniline

To a solution of 3-nitrobenzenamine (5 g, 36.2 mmol) in acetone (30 mL) were added iodomethane (5.65 g, 39.8 mmol) and potassium carbonate (10 g, 72.4 mmol). The mixture was stirred at 50° C. overnight. The mixture was concentrated to dryness under reduced pressure. The residue was purified by column chromatography (petroleum ether:ethylacetate, 10:1) to give N-methyl-3-nitroaniline (1.6 g, 29%) as a red solid.

LCMS (Method B): 3.01 min [MH]⁺=153.1.

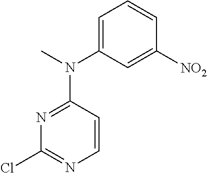 [SMILES: CN(c1cccc([N+](=O)[O-])c1)c1ccnc(Cl)n1]

### Step 2: 2-chloro-N-methyl-N-(3-nitrophenyl)pyrimidin-4-amine

To a solution of N-methyl-3-nitrobenzenamine (1.6 g, 10.5 mmol) in N,N-Dimethyl formamide (10 mL) were added 2,4-dichloropyridine (1.72 g, 11.6 mmol) and potassium carbonate (2.18 g, 15.8 mmol). The mixture was stirred at 130° C. for 3 hours. The reaction mixture was then partitioned between ethyl acetate and water, the organic layer was separated and washed with water, brine, dried over sodium sulfate and concentrated to give a residue which was purified by column chromatography (petroleum ether:ethylacetate, 5:1) to give 2-chloro-N-methyl-N-(3-nitrophenyl)pyrimidin-4-amine (1.0 g, 36%) as a light yellow solid. LCMS (Method B): 3.01 min [MH]⁺=265.1.

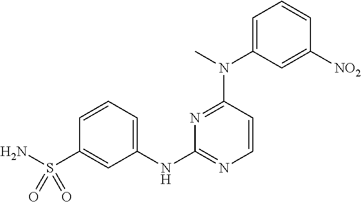 [SMILES: CN(c1cccc([N+](=O)[O-])c1)c1ccnc(Nc2cccc(S(N)(=O)=O)c2)n1]

### Step 3: 3-((4-(methyl(3-nitrophenyl)amino)pyrimidin-2-yl)amino)benzene-sulfonamide

To a solution of 2-chloro-N-methyl-N-(3-nitrophenyl)pyrimidin-4-amine (1 g, 3.78 mmol) in 1,4-dioxane (20 mL) were added 3-aminobenzenesulfonamide (683 mg, 3.97 mmol) and p-toluenesulfonic acid monohydrate (575 mg, 3.02 mmol). The mixture was stirred at 120° C. for 3 hours. The solvent was removed under reduced pressure and ammonia solution (30 mL) was added to form a precipitate. The solid was collected via filtration and dried under reduced pressure to give 3-((4-(methyl(3-nitrophenyl)amino)pyrimidin-2-yl)amino)benzenesulfonamide (1.4 g, 93% yield) a brown solid. LCMS (Method B): 1.24 min [MH]⁺=401.1.

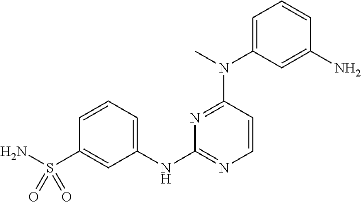 [SMILES: CN(c1cccc(N)c1)c1ccnc(Nc2cccc(S(N)(=O)=O)c2)n1]

### Step 4: 3-((4-((3-aminophenyl)(methyl)amino)pyrimidin-2-yl)amino)benzene sulfonamide

To a solution of 3-((4-(methyl(3-nitrophenyl)amino)pyrimidin-2-yl)amino)benzene sulfonamide (700 mg, 1.75 mmol) in methanol (30 mL) were added zinc powder (3.0 g) and saturated ammonium chloride aqueous solution (30 mL). The mixture was stirred at room temperature overnight. The solid was filtered off and the solvent was removed under reduced pressure to give a residue which was partitioned between water and ethylacetate. The organic phase was separated and washed with water, brine, dried over sodium sulfate and concentrated under reduced pressure to give 3-((4-((3-aminophenyl)(methyl)amino)pyrimidin-2-yl)amino)benzene sulfonamide (600 mg, 92%) as a light yellow solid. LCMS (Method B): 0.49 min [MH]⁺=371.1.

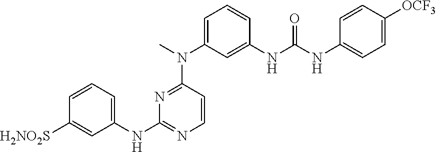 [SMILES: CN(c1cccc(NC(=O)Nc2ccc(OC(F)(F)F)cc2)c1)c1ccnc(Nc2cccc(S(N)(=O)=O)c2)n1]

### Step 5: 3-((4-(methyl(3-(3-(4-(trifluoromethoxy)phenyl) ureido)phenyl)amino)pyrimidin-2-yl)amino) benzenesulfonamide

3-((4-(Methyl(3-(3-(4-(trifluoromethoxy)phenyl)ureido)phenyl)amino)pyrimidin-2-yl)amino) benzenesulfonamide. To a solution of 3-((4-((3-aminophenyl)(methyl)amino) pyrimidin-2-yl)amino)benzene sulfonamide (200 mg, 0.54 mmol) in tetrahydrofuran (20 mL) were added 4-(trifluoromethoxy)phenyl isocyanate (121 mg, 0.54 mmol) and N,N-diisopropylethylamine (140 mg, 1.08 mmol). The mixture was stirred at room temperature for 3 hours. The reaction mixture was concentrated under reduced pressure to give a residue which was purified by silica-gel chromatography (dichloromethane/methanol, 35:1) to give 3-((4-(methyl(3-(3-(4-(trifluoromethoxy)phenyl) ureido)phenyl)amino)pyrimidin-2-yl)amino) benzenesulfonamide (190 mg, 65%) as a light brown solid. ¹H NMR (400 MHz, DMSO-d₆): δ ppm 3.47 (s, 3H), 5.89 (d, J=6.0 Hz, 1H), 6.97 (d, J=8.0 Hz, 1H), 7.32-7.24 (m, 4H), 7.44-7.33 (m, 4H), 7.55 (s, 1H), 7.55 (d, J=8.8 Hz, 2H), 7.81 (d, J=8.0 Hz, 1H), 7.91 (d, J=6.0 Hz, 1H), 8.56 (s, 1H), 9.07 (s, 1H), 9.10 (s, 1H), 9.57 (s, 1H). LCMS (Method B): 2.50 min [MH]⁺=574.2, [MNa]⁺=596.2.

Compound 15

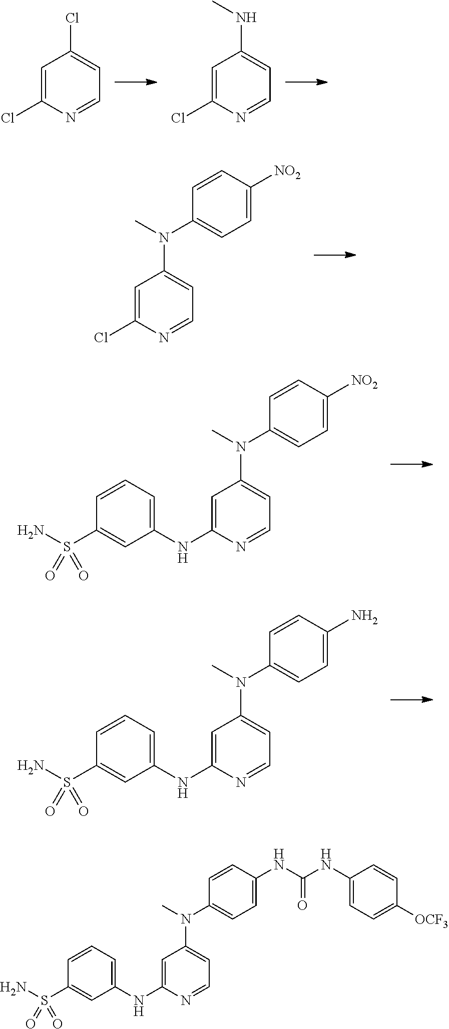

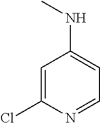

### Step 1: 2-chloro-N-methylpyridin-4-amine

To a sealed tube were added 2, 4-dichloropyridine (2 g, 13.5 mmol) and 2 M methylamine in methanol. The mixture was heated to 85° C. overnight. The solvent was removed and the crude was purified by column chromatography (petroleum ether/ethylacetate, 3:1) to give 2-chloro-N-methylpyridin-4-amine (1.58 g, 82%) as a white solid. LCMS (Method B): 1.54 min [MH]⁺=143.0.

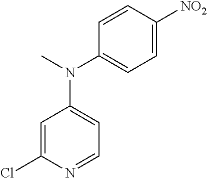 [SMILES: CN(c1ccc([N+](=O)[O-])cc1)c1ccnc(Cl)c1]

### Step 2: 2-chloro-N-methyl-N-(4-nitrophenyl)pyridin-4-amine

2-Chloro-N-methylpyridin-4-amine (200 mg, 1.4 mmol), 4-Fluoro-nitrobenzene (217 mg, 1.54 mmol) and K₂CO₃ (368 mg, 2.8 mmol) were added to DMSO (5 mL). The reaction mixture was heated to 120° C. until all starting materials were disappeared. The reaction mixture was poured into water (20 mL). The solid was collected by filtration, dried to give 2-chloro-N-methyl-N-(4-nitrophenyl)pyridin-4-amine (280 mg, 75.9%) as a yellow solid. LCMS (Method B): 2.37 min [MH]⁺=264.0.

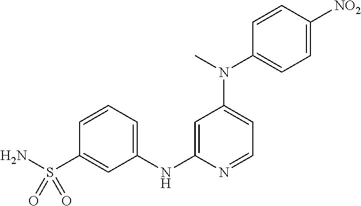 [SMILES: CN(c1ccc([N+](=O)[O-])cc1)c1ccnc(Nc2cccc(S(N)(=O)=O)c2)c1]

### Step 3: 3-((4-(methyl(4-nitrophenyl)amino)pyridin-2-yl)amino)benzene-sulfonamide

2-Chloro-N-methyl-N-(4-nitrophenyl)pyridin-4-amine (400 mg, 1.52 mmol), 3-aminobenzamide (261 mg, 1.52 mmol), Cs₂C03 (991 mg, 3.04 mmol), xantphos (174 mg, 0.3 mmol) were added in 1,4-dioxane (15 mL), followed by addition of Pd₂(dba)₃ (139 mg, 0.152 mmol). The reaction mixture was heated to 120° C. overnight under nitrogen. The mixture was concentrated in vacuo and the crude was purified by column chromatography (DCM/methanol, 100:0 to 98:2) to give 3-((4-(methyl(4-nitrophenyl)amino)pyridin-2-yl)amino)benzenesulfonamide (303 mg, 50%) as a yellow solid. LCMS (Method B): 0.75 min [MH]⁺=400.1.

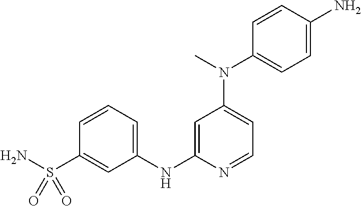 [SMILES: CN(c1ccc(N)cc1)c1ccnc(Nc2cccc(S(N)(=O)=O)c2)c1]

### Step 4: 3-((4-((4-aminophenyl)(methyl)amino)pyridin-2-yl)amino) benzenesulfonamide

3-((4-((4-Aminophenyl)(methyl)amino)pyridin-2-yl)amino)benzenesulfonamide (300 mg, 0.75 mmol) was dissolved in the mixed solvent of DMF (30 mL) and saturated NH₄Cl (30 mL), followed by addition of zinc powder (487 mg, 7.5 mmol). The reaction mixture was stirred at room temperature overnight. The solid was removed by filtration, washed three times with methanol (30 mL). The filtrates were collected and concentrated to give a residue. The crude was washed three times with water (30 mL). The yellow solid was collected and dried to give 3-((4-((4-aminophenyl)(methyl)amino)pyridin-2-yl)amino) benzenesulfonamide (200 mg, 72%). LCMS (Method B): 0.35 min [MH]⁺=370.1.

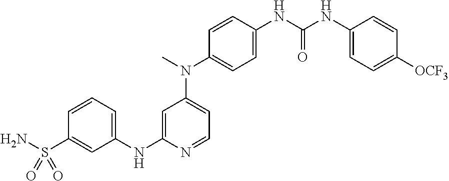 [SMILES: CN(c1ccc(NC(=O)Nc2ccc(OC(F)(F)F)cc2)cc1)c1ccnc(Nc2cccc(S(N)(=O)=O)c2)c1]

### Step 5: 3-((4-(methyl(4-(3-(4-(trifluoromethoxy)phenyl)ureido)phenyl)amino)pyridin-2-yl)amino) benzenesulfonamide

3-((4-(Methyl(4-(3-(4-(trifluoromethoxy)phenyl)ureido)phenyl)amino)pyridin-2-yl)amino) benzenesulfonamide. To a solution of 3-((4-((4-aminophenyl)(methyl)amino)pyridin-2-yl)amino) benzenesulfonamide (200 mg, 0.54 mmol) in THF (10 mL) were added 1-isocyanato-4-(trifluoromethoxy)benzene (115 mg, 0.57 mmol) and DIEA (140 mg, 1.08 mmol). The reaction was stirred at room temperature overnight. The mixture was concentrated in vacuo and the crude was purified by column chromatography (DCM/methanol, 100:0 to 97.5:2.5) to give 3-((4-(methyl(4-(3-(4-(trifluoromethoxy) phenyl)ureido)phenyl)amino)pyridin-2-yl)amino) benzenesulfonamide (120 mg, 39%) as a yellow solid. ¹H NMR (400 MHz, DMSO-d₆): δ ppm 3.24 (s, 3H), 6.03 (d, J=2.0 Hz, 1H), 6.20 (dd, J=6.0 and 2.1 Hz, 1H), 7.39-7.18 (m, 8H), 7.57 (t, J=9.5 Hz, 4H), 7.86-7.77 (m, 2H), 8.21 (s, 1H), 9.04 (br s, 2H), 9.12 (s, 1H). LCMS (Method B): 2.43 min [MH]⁺=573.2, [MNa]⁺=595.2.

Compound 16

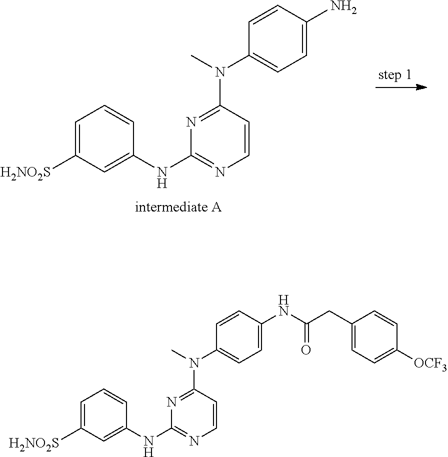 [SMILES: CN(c1ccc(N)cc1)c1ccnc(Nc2cccc(S(N)(=O)=O)c2)n1 ; CN(c1ccc(NC(=O)Cc2ccc(OC(F)(F)F)cc2)cc1)c1ccnc(Nc2cccc(S(N)(=O)=O)c2)n1]

### Step 1; N-(4-(methyl(2-((3-sulfamoylphenyl)amino)pyrimidin-4-yl)amino) phenyl)-2-(4-(trifluoro-methoxy)phenyl)acetamide

2-(4-(Trifluoromethoxy)phenyl)acetic acid (63 mg, 0.28 mmol), intermediate A (100 mg, 0.27 mmol) and DIEA (70 mg, 0.54 mmol) were added in DMF (3 mL). HATU (123 mg, 0.324 mmol) was then added. The reaction mixture was stirred at room temperature overnight. The crude was purified by column chromatography with eluent (DCM/methanol, 100:0 to 97:3) to give a white solid which was purified by preparative HPLC to give N-(4-(methyl(2-((3-sulfamoylphenyl)amino)pyrimidin-4-yl)amino)phenyl)-2-(4-(trifluoro-methoxy)phenyl)acetamide (15 mg, 10%) as a white solid. ¹H NMR (400 MHz, CDCl₃): 53.55 (s, 3H), 3.70 (s, 2H), 5.82 (s, 1H), 6.18 (s, 1H), 7.17 (m, 4H), 7.38 (d, J=8.5 Hz, 2H), 7.50 (br s, 2H), 7.68 (m, 3H), 7.84 (br s, 1H), 8.41 (s, 1H). LCMS (Method B): 2.42 min [MH]⁺=573.2.

Compound 17

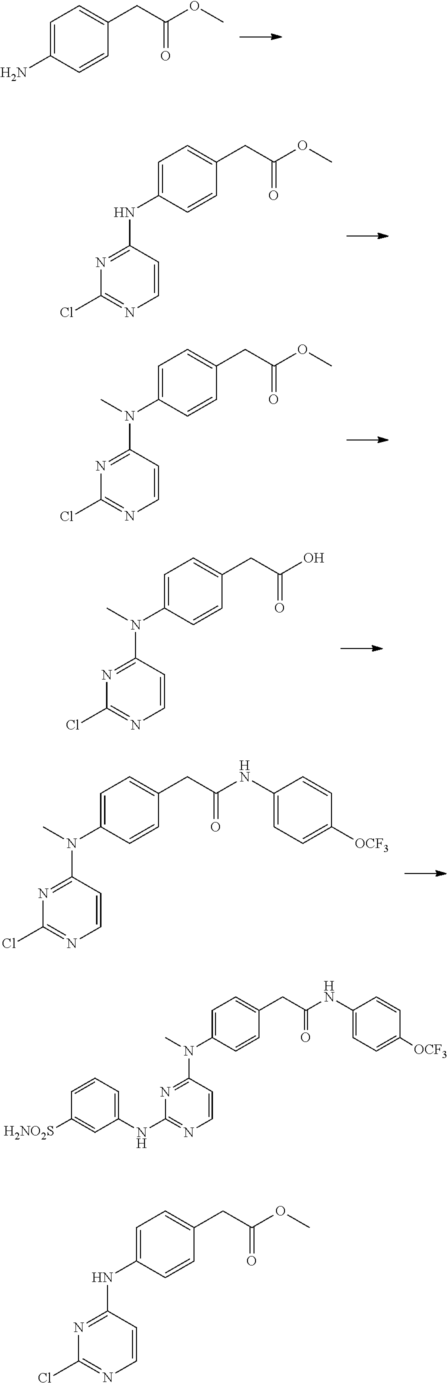

### Step 1: Methyl 2-(4-((2-chloropyrimidin-4-yl)amino)phenyl)acetate

DIEA (2.2 g, 16.8 mmol) was added slowly to a solution of 2,4-dichloropyrimine (500 mg, 3.36 mmol) and methyl 2-(4-aminophenyl)acetate (664 mg, 4 mmol) in ethanol (20 mL) at 10° C. Thereafter, the mixture was heated at reflux for 48 hours. The mixture was concentrated and purified by column chromatography (petroleum ether/ethylacetate, 3:1) to give methyl 2-(4-((2-chloropyrimidin-4-yl)amino)phenyl)acetate (740 mg, 79%) as a red oil. LCMS (Method B): 2.37 min [MH]⁺=278.0.

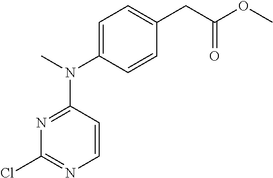 [SMILES: COC(=O)Cc1ccc(N(C)c2ccnc(Cl)n2)cc1]

### Step 2: Methyl 2-(4-((2-chloropyrimidin-4-yl)(methyl)amino)phenyl)acetate

Methyl 2-(4-((2-chloropyrimidin-4-yl)amino)phenyl)acetate (490 mg, 1.76 mmol) was dissolved in DMF (6 mL) and cesium carbonate (1.7 g, 5.3 mmol). After 15 min, methyl iodide (376 mg, 2.65 mmol) was added and the mixture was stirred at room temperature for 16 hours. Water was added to quench the reaction and the resulting mixture was extracted with ethylacetate. The combined organic layer was dried over sodium sulfate, concentrated and purified by column chromatography (petroleum ether/ethylacetate, 7:1) to give methyl 2-(4-((2-chloropyrimidin-4-yl)(methyl)amino)phenyl)acetate (260 mg, 61%) as yellow oil. ¹H NMR (400 MHz, DMSO-d₆): δ ppm 3.38 (s, 3H), 3.65 (s, 3H), 3.77 (s, 2H), 6.30 (d, J=6.0 Hz, 1H), 7.35 (m, 2H), 7.42 (d, J=8.4 Hz, 2H), 8.00 (d, J=6.0 Hz, 1H). LCMS (Method B): 2.48 min [MH]⁺=292.1.

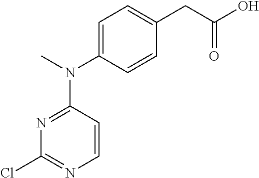 [SMILES: CN(c1ccc(CC(=O)O)cc1)c1ccnc(Cl)n1]

### Step 3: 2-(4-((2-chloropyrimidin-4-yl)(methyl)amino)phenyl)acetic acid

To a solution of methyl 2-(4-((2-chloropyrimidin-4-yl)(methyl)amino)phenyl)acetate (320 mg, 1.1 mmol) in THF (16 mL) and methanol (8 mL) was added 1 M sodium hydroxide (4.8 mL). The reaction mixture was stirred at room temperature for 2 hours. The mixture was adjusted to pH=2 with 1M HCl solution and extracted with ethylacetate several times. The combined organic layers were dried over sodium sulfate, concentrated to give 2-(4-((2-chloropyrimidin-4-yl)(methyl)amino)phenyl)acetic acid (304 mg, quantitative) as a white solid. LCMS (Method B): 2.32 min [MH]⁺=278.2.

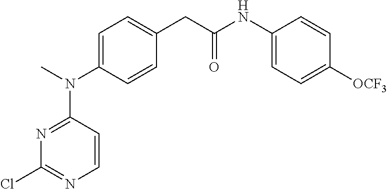 [SMILES: CN(c1ccc(CC(=O)Nc2ccc(OC(F)(F)F)cc2)cc1)c1ccnc(Cl)n1]

### Step 4: 2-(4-((2-chloropyrimidin-4-yl)(methyl)amino)phenyl)-N-(4-(trifluoromethoxy) phenyl)acetamide

A mixture of 2-(4-((2-chloropyrimidin-4-yl)(methyl)amino)phenyl)acetic acid (304 mg, 1.1 mmol) in dichloromethane (8 mL) was stirred at room temperature. 4-(trifluoromethoxy) aniline (195 mg, 1.1 mmol) and TEA (222 mg, 2.2 mmol) were added, followed by addition of EDC.HCl (211 mg, 1.1 mmol), and HOBt (148 mg, 1.1 mmol). The mixture was stirred at room temperature for 16 hours. The mixture was concentrated and purified by column chromatography (petroleum ether/ethylacetate, 3:1 to 1:1) to give 2-(4-((2-chloropyrimidin-4-yl)(methyl)amino)phenyl)-N-(4-(trifluoro-methoxy) phenyl) acetamide (380 mg, 79%) as a white solid. LCMS (Method B): 2.97 min [MH]⁺=437.1.

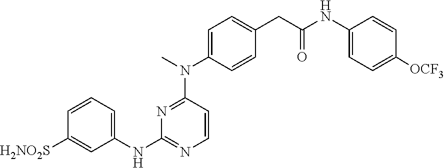 [SMILES: CN(c1ccc(CC(=O)Nc2ccc(OC(F)(F)F)cc2)cc1)c1ccnc(Nc2cccc(S(N)(=O)=O)c2)n1]

### Step 5: 2-(4-(methyl(2-((3-sulfamoylphenyl)amino)pyrimidin-4-yl)amino)phenyl)-N-(4-(trifluoromethoxy)phenyl)acetamide

2-(4-(Methyl(2-((3-sulfamoylphenyl)amino)pyrimidin-4-yl)amino)phenyl)-N-(4-(trifluoro-methoxy)phenyl)acetamide. 2-(4-((2-chloropyrimidin-4-yl)(methyl)amino)phenyl)-N-(4-(trifluoromethoxy)phenyl) acetamide (100 mg, 0.23 mmol) and 3-aminophenylsulfamide (39 mg, 0.23 mmol) were dissolved in DMF (3 mL). To this mixture was added p-TsOH.H₂O (85 mg, 0.23 mmol). The reaction mixture was stirred at 60° C. for 16 hours. The mixture was then adjusted to pH=9 with a sodium carbonate solution. The resulting mixture was extracted with ethylacetate several times. The combined extract was dried over sodium sulfate, concentrated and purified by column chromatography (petroleum ether/ethylacetate, 30:1) to give 2-(4-(methyl(2-((3-sulfamoylphenyl)amino)pyrimidin-4-yl)amino)phenyl)-N-(4-(trifluoromethoxy)phenyl) acetamide (100 mg, 76%) as a white solid. ¹H NMR (400 MHz, DMSO-d₄): δ ppm 3.45 (s, 3H), 3.72 (s, 2H), 5.83 (d, J=6.0 Hz, 1H), 7.27 (s, 2H), 7.33 (m, 5H), 7.39 (t, J=7.9 Hz, 1H), 7.46 (d, J=8.3 Hz, 2H), 7.74 (d, J=8.8 Hz, 2H), 7.79 (d, J=8.3 Hz, 1H), 7.89 (d, J=6.0 Hz, 1H), 8.54 (s, 1H), 9.55 (s, 1H), 10.45 (s, 1H). LCMS (Method B): 2.32 min [MH]⁺=573.2, [MNa]⁺=595.2.

Compound 18

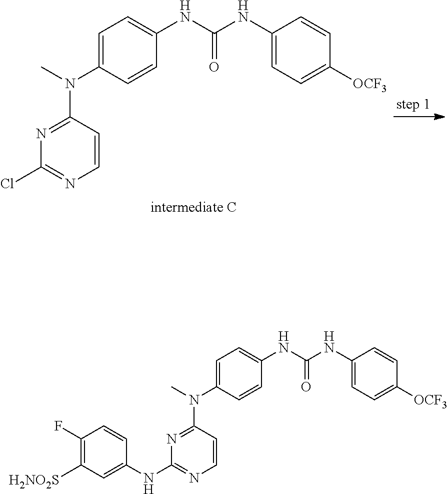 [SMILES: CN(c1ccc(NC(=O)Nc2ccc(OC(F)(F)F)cc2)cc1)c1ccnc(Cl)n1 ; CN(c1ccc(NC(=O)Nc2ccc(OC(F)(F)F)cc2)cc1)c1ccnc(Nc2ccc(F)c(S(N)(=O)=O)c2)n1]

### Step 1: 2-Fluoro-5-((4-(methyl(4-(3-(4-(trifluoromethoxy)phenyl)ureido) phenyl)amino) pyrimidin-2-yl)amino)benzenesulfonamide

Following general procedure C with intermediate B (60 mg, 0.14 mmol) and 3-amino-4-fluorosulfonamide (26 mg, 0.14 mmol), 2-fluoro-5-((4-(methyl(4-(3-(4-(trifluoromethoxy) phenyl)ureido)phenyl)amino)pyrimidin-2-yl)amino)benzene-sulfonamide (50 mg, 60%) was obtained as a white solid. ¹H NMR (400 MHz, DMSO-d₄): δ 3.60 (s, 3H), 5.93 (s, 1H), 7.34 (m, 5H), 7.69 (s, 2H), 7.61 (m, 4H), 7.96-7.74 (m, 2H), 8.36 (s, 1H), 9.32 (s, 2H), 10.49 (s, 1H). LCMS (Method B): 2.50 min [MH]⁺=592.2, [MNa]⁺=614.1.

Compound 19

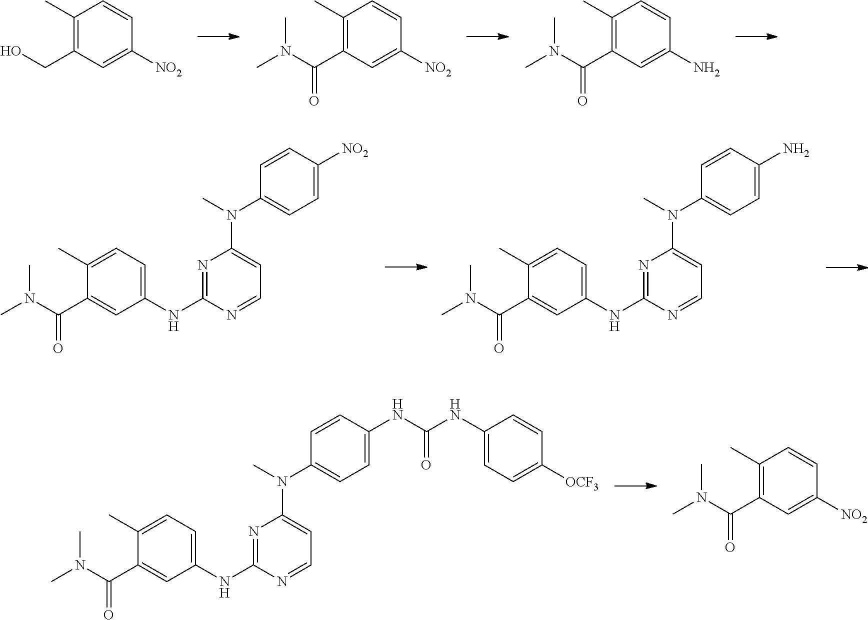

### Step 1: N,N,2-trimethyl-5-nitrobenzamide

2-Methyl-5-nitrobenzoic acid (1 g, 5.5 mmol) was dissolved in SOCl₂ (15 mL), followed by addition of DMF (1 drop). The reaction mixture was refluxed for 4 hours. The solvent was removed under reduced pressure. DCM (10 mL) was added and then the reaction mixture was concentrated under reduced pressure. The DCM addition/concentration cycle was repeated three times to give a white solid. Me₂NH.HCl (490 mg, 6.08 mmol) and TEA (1.67 g, 16.6 mmol) were dissolved in DCM (20 mL). 2-methyl-5-nitrobenzoyl chloride (1.09 g, 5.5 mmol) in DCM (5 mL) was added to the mixture slowly at 0° C. The reaction mixture was stirred at room temperature for 16 hours, washed with water (3×30 mL) and the organics was dried (Na₂SO₄) and concentrated to give N,N,2-trimethyl-5-nitrobenzamide (600 mg, 52.2%) as a white solid. LCMS (Method B): 1.06 min [MH]⁺=208.4.

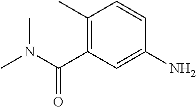

### Step 2: 5-amino-N,N,2-trimethylbenzamide

N,N,2-Trimethyl-5-nitrobenzamide (300 mg, 1.44 mmol) was dissolved in methanol (20 mL), followed by addition of wet Pd/C (10%, 30 mg). The reaction mixture was stirred overnight under hydrogen. The catalyst was filtered off by filtration and the filtrate was concentrated under reduced pressure to give 5-amino-N,N,2-trimethylbenzamide (180 mg, 70%) as a yellow solid which was used in next step directly. LCMS (Method B): 0.30 min [MH]⁺=179.1.

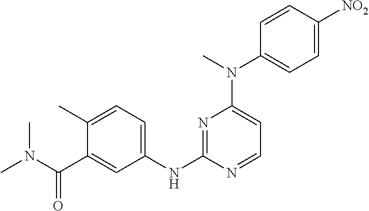 [SMILES: Cc1ccc(Nc2nccc(N(C)c3ccc([N+](=O)[O-])cc3)n2)cc1C(=O)N(C)C]

### Step 3: N,N,2-trimethyl-5-((4-(methyl(4-nitrophenyl)amino)pyrimidin-2-yl)amino) benzamide

2-Chloro-N-methyl-N-(4-nitrophenyl)pyrimidin-4-amine (from step 2 of preparation of intermediate A, 223 mg, 0.84 mmol) and 5-amino-N,N,2-trimethylbenzamide (150 mg, 0.84 mmol) were dissolved in 1,4-dioxane (4 mL), followed by addition of concentrated HCl (2 drops). The reaction mixture was heated to 120° C. for 3 hours. The solvent was removed and the crude product was washed with petroleum ether/DCM, 1:1 to give N,N,2-trimethyl-5-((4-(methyl(4-nitrophenyl)amino)pyrimidin-2-yl)amino)benzamide (250 mg, 73%) as a yellow solid which was used in next step without further purification.

LCMS (Method B): 1.96 min [MH]⁺=407.2.

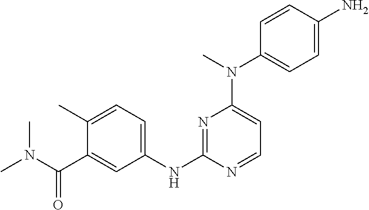 [SMILES: Cc1ccc(Nc2nccc(N(C)c3ccc(N)cc3)n2)cc1C(=O)N(C)C]

### Step 4: 5-((4-((4-aminophenyl)(methyl)amino)pyrimidin-2-yl)amino)-N,N,2-trimethylbenzamide

To a solution of N,N,2-trimethyl-5-((4-(methyl(4-nitrophenyl)amino)pyrimidin-2-yl)amino)benzamide (250 mg, 0.62 mmol) in methanol (10 mL) were added zinc (400 mg, 6.2 mmol) and saturated aqueous solution of NH₄Cl (10 mL). The reaction mixture was stirred at room temperature overnight. The solid was removed by filtration, washed three times with methanol (20 mL). The filtrates were collected and the organic solvent was removed to give a residue which was extracted with DCM (3×50 mL). The combined organic layers were washed with water and dried over Na₂SO₄. The solvent was removed under reduced pressure to give 5-((4-((4-aminophenyl)(methyl)amino) pyrimidin-2-yl)amino)-N,N,2-trimethylbenzamide (150 mg, 64%) as a yellow solid. LCMS (Method B): 1.12 min [MH]⁺=377.2.

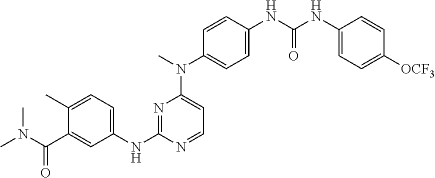 [SMILES: Cc1ccc(Nc2nccc(N(C)c3ccc(NC(=O)Nc4ccc(OC(F)(F)F)cc4)cc3)n2)cc1C(=O)N(C)C]

### Step 5: N,N,2-trimethyl-5-((4-(methyl(4-(3-(4-(trifluoromethoxy)phenyl)ureido) phenyl)amino) pyrimidin-2-yl)amino)benzamide

To a solution of 5-((4-((4-aminophenyl)(methyl)amino)pyrimidin-2-yl)amino)-N,N,2-trimethylbenzamide (150 mg, 0.40 mmol) in THF (15 mL) were added 1-isocyanato-4-(trifluoromethoxy)benzene (80 mg, 0.40 mmol) and DIEA (155 mg, 1.2 mmol). The reaction mixture was stirred at room temperature overnight. The mixture was concentrated in vacuo and the crude product was purified by column chromatography (DCM:methanol, 100 to 100:2) to give N,N,2-trimethyl-5-((4-(methyl(4-(3-(4-(trifluoromethoxy)phenyl)ureido) phenyl)amino) pyrimidin-2-yl)amino)benzamide (82 mg, 35%) as a yellow solid. ¹H NMR (400 MHz, DMSO-d₄): δ 2.16 (m, 3H), 2.77 (m, 3H), 3.01 (m, 3H), 3.44 (m, 3H), 6.00-6.37 (m, 1H), 7.89-7.21 (m, 12H), 9.32 (s, 1H), 9.50 (s, 1H), 10.56-10.78 (m, 1H). LCMS (Method B): 2.54 min [MH]⁺=580.3.

Compound 20

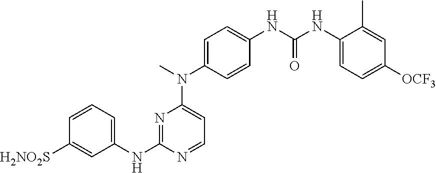 [SMILES: Cc1cc(OC(F)(F)F)ccc1NC(=O)Nc1ccc(N(C)c2ccnc(Nc3cccc(S(N)(=O)=O)c3)n2)cc1]

### 3-((4-(Methyl(4-(3-(2-methyl-4-(trifluoromethoxy)phenyl)ureido)phenyl)-amino)pyrimidin-2-yl)amino) benzenesulfonamide

Following general procedure A using intermediate A (60 mg, 0.12 mmol) and 2-methyl-4-trifluoromethoxyaniline (24 mg, 0.13 mmol), 3-((4-(Methyl(4-(3-(2-methyl-4-(trifluoro-methoxy)phenyl)ureido)phenyl)amino)pyrimidin-2-yl)amino)benzenesulfonamide (4 mg, 4%) was obtained as a white solid after purification by column chromatography (DCM/MeOH, 100:0 to 98:2). ¹H NMR (400 MHz, MeOD-d₄): δ ppm 2.25 (s, 3H), 3.32 (s, 3H), 5.86 (d, J=6.8 Hz, 1H), 7.03 (m, 2H), 7.12 (d, J=8.4 Hz, 2H), 7.65 (m, 6H), 7.83 (m, 2H), 8.41 (s, 1H). LCMS (Method B): 2.48 min [MH]⁺=588.2, [MNa]⁺=610.2.

Compound 21

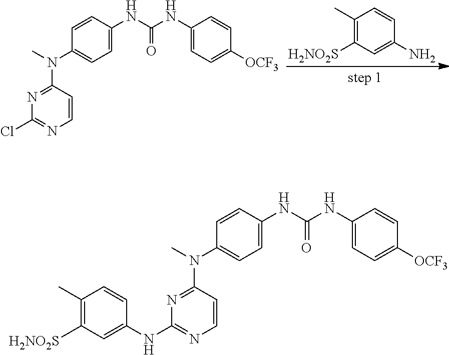 [SMILES: CN(c1ccc(NC(=O)Nc2ccc(OC(F)(F)F)cc2)cc1)c1ccnc(Cl)n1 ; Cc1ccc(N)cc1S(N)(=O)=O ; Cc1ccc(Nc2nccc(N(C)c3ccc(NC(=O)Nc4ccc(OC(F)(F)F)cc4)cc3)n2)cc1S(N)(=O)=O]

### Step 1; 2-Methyl-5-((4-(methyl(4-(3-(4-(trifluoromethoxy)phenyl)ureido)phenyl)amino) pyrimidin-2-yl)amino) benzenesulfonamide

Following general procedure C using intermediate A (100 mg, 0.43 mmol) and 3-amino-4-methylbenzenesulfonamide (43 mg, 0.23 mmol) 2-Methyl-5-((4-(methyl(4-(3-(4-(trifluoromethoxy)phenyl)ureido)phenyl)amino)pyrimidin-2-yl)amino)benzene sulfonamide (100 mg, 75%) was obtained as a white solid. ¹H NMR (400 MHz, DMSO-d₆): δ ppm 2.56 (s, 3H), 3.50 (s, 3H), 5.99 (m, 1H), 7.38 (m, 7H), 7.65 (m, 5H), 7.87 (d, J=7.2 Hz, 1H), 8.36 (s, 1H), 9.24 (d, J=3.6 Hz, 2H), 10.50 (s, 1H). LCMS (Method B): 2.52 min [MH]⁺=588.2.

Compound 22

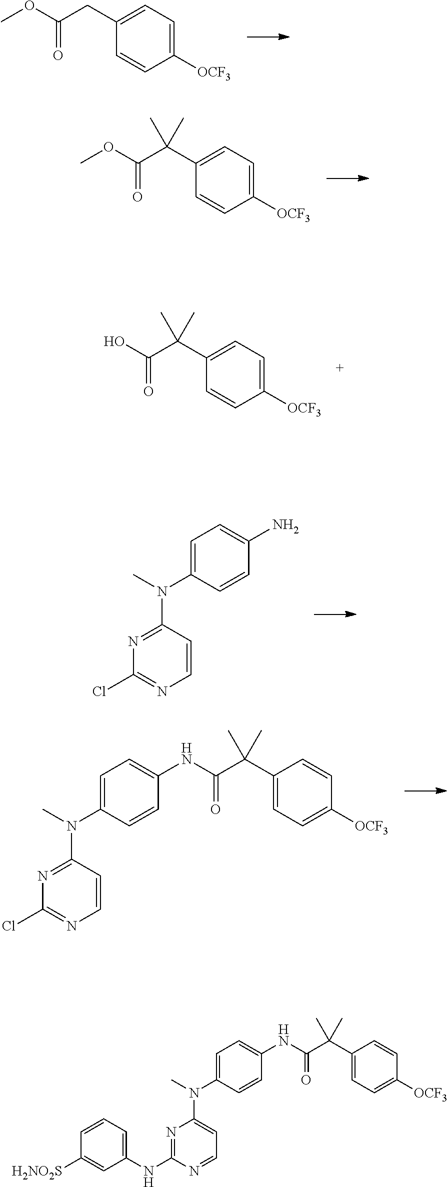

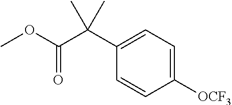 [SMILES: COC(=O)C(C)(C)c1ccc(OC(F)(F)F)cc1]

### Step 1: Methyl 2-methyl-2-(4-(trifluoromethoxy)phenyl)propanoate

To a mixture of NaH (60%, 362 mg, 9.1 mmol) in DMF (8 mL) was added methyl 2-(4-(trifluoromethoxy)phenyl)acetate (530 mg, 2.26 mmol). The mixture was stirred at 0° C. for 30 min, and then methyl iodide (1.29 g, 9.1 mmol) was added. The reaction was stirred at rt overnight. Another batch was repeated beginning with 106 mg of methyl 2-(4-(trifluoromethoxy)phenyl)acetate. The two batches were combined and poured into water. The resulting mixture was extracted with ethylacetate. The organic layer was dried over sodium sulfate, concentrated and purified by column chromatography (petroleum ether/ethylacetate, 30:1) to give methyl 2-methyl-2-(4-(trifluoromethoxy)phenyl)propanoate as a yellow oil (440 mg, 60%). LCMS (Method B): 2.85 min [MH]⁺=263.1.

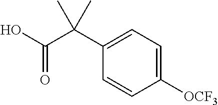 [SMILES: CC(C)(C(=O)O)c1ccc(OC(F)(F)F)cc1]

### Step 2: 2-methyl-2-(4-(trifluoromethoxy) phenyl)propanoic acid

Sodium hydroxide solution (1 M, 1.5 mL) was added to the solution of methyl 2-methyl-2-(4-(trifluoromethoxy)phenyl)propanoate (50 mg, 0.19 mmol) in methanol (1.0 mL) and THF (2.0 mL). The reaction mixture was stirred at rt overnight and then HCl solution (1 M) was added to make the pH=1-2. The resulting mixture was extracted with ethylacetate. The organic layer was dried over sodium sulfate and concentrated to give 2-methyl-2-(4-(trifluoromethoxy) phenyl)propanoic acid (42 mg, 89%) as a brown oil.

LCMS (Method B): 2.80 min [MH]⁺=271.0.

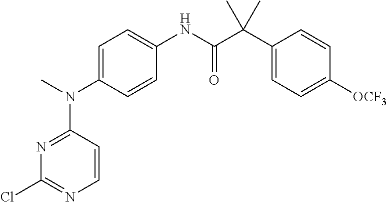 [SMILES: CN(c1ccc(NC(=O)C(C)(C)c2ccc(OC(F)(F)F)cc2)cc1)c1ccnc(Cl)n1]

### Step 3: 2-methyl-N-(4-(methyl(2-((3-sulfamoylphenyl)amino)pyrimidin-4-yl)amino) phenyl)-2-(4-(trifluoromethoxy)phenyl)propanamide

2-methyl-2-(4-(trifluoromethoxy)phenyl)propanoic acid (270 mg, 1.09 mmol) was dissolved in SOCl₂ (4 mL) under nitrogen, followed by addition of DMF (1 drop). The resulting mixture was stirred at 80° C. for 3 hours. The excess SOCl₂ was evaporated and the resulting residue was dissolved in DCM (6 mL). The solution was slowly added to a solution of N1-(2-chloropyrimidin-4-yl)-N1-methylbenzene-1,4-diamine (from step 2 of the preparation of Intermediate C, 255 mg, 1.09 mmol) and TEA (275 mg, 2.72 mmol) in DCM (6 mL) at 0° C. The reaction was stirred at room temperature overnight. The mixture was then partitioned between a 1 M HCl solution and ethylacetate. The organic layer was separated, dried over sodium sulfate and concentrated to give a residue which was purified by column chromatography (DCM/MeOH, 20:1) to generate desired intermediate (50 mg, 10%) as a yellow solid. LCMS (Method B): 3.14 min [MH]+=465.1.

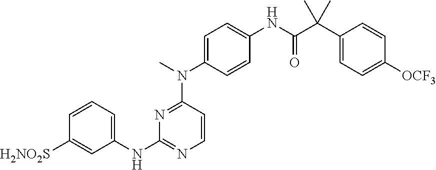 [SMILES: CN(c1ccc(NC(=O)C(C)(C)c2ccc(OC(F)(F)F)cc2)cc1)c1ccnc(Nc2cccc(S(N)(=O)=O)c2)n1]

### Step 4

### 2-methyl-N-(4-(methyl(2-((3-sulfamoylphenyl)amino)pyrimidin-4-yl)amino)phenyl)-2-(4-(trifluoromethoxy)phenyl)propanamide

Following general procedure C using 2-methyl-N-(4-(methyl(2-((3-sulfamoylphenyl)amino)pyrimidin-4-yl)amino)phenyl)-2-(4-(trifluoromethoxy)phenyl)propanamide (step 3) (50 mg, 0.107 mmol) and 3-aminobenzenesulfonamide (18 mg, 0.107 mmol), 2-methyl-N-(4-(methyl(2-((3-sulfamoylphenyl)amino)pyrimidin-4-yl)amino)phenyl)-2-(4-(trifluoro-methoxy)phenyl)propanamide (30 mg, 47%) was obtained as a white solid. ¹H NMR (400 MHz, DMSO-d₆): δ ppm 1.60 (s, 6H), 3.47 (s, 3H), 5.94 (br s, 1H), 7.38 (m, 6H), 7.44 (m, 6H), 7.52 (m, 4H), 7.79 (m, 3H), 7.88 (d, J=6.8 Hz, 1H), 8.39 (s, 1H), 9.41 (s, 1H), 10.07 (s, 1H), 10.40 (s, 1H). LCMS (Method B): 2.52 min [MH]⁺=601.2.

Compound 23

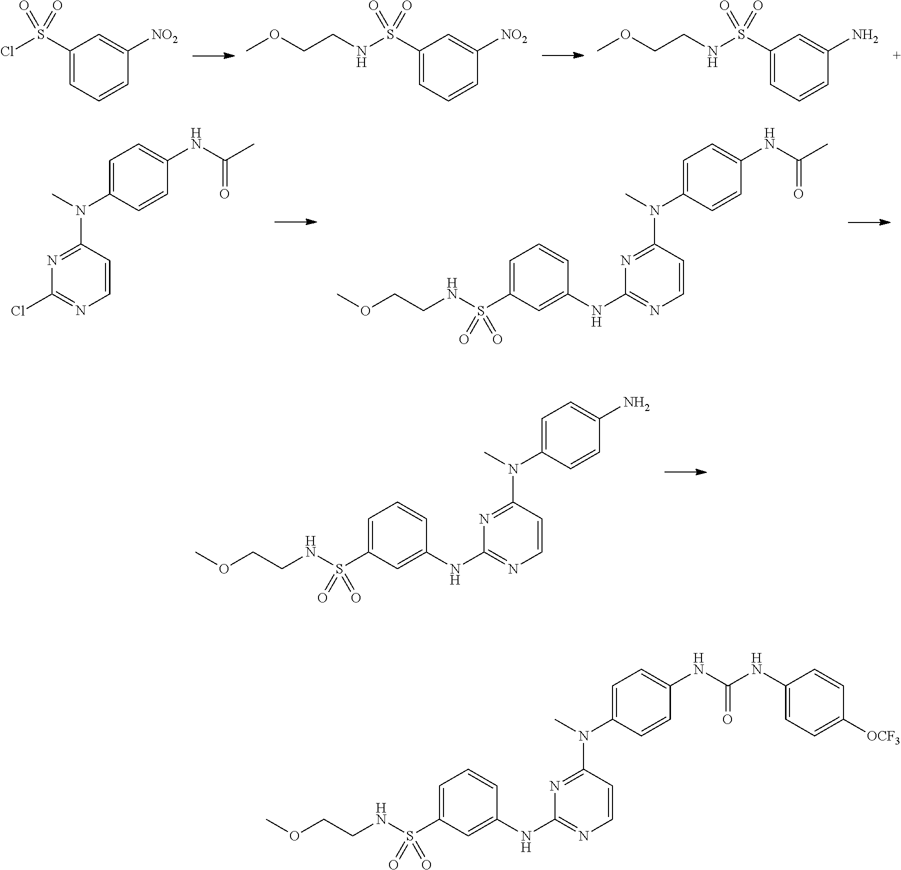

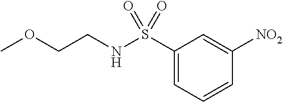 [SMILES: COCCNS(=O)(=O)c1cccc([N+](=O)[O-])c1]

### Step 1: N-(2-Methoxyethyl)-3-nitrobenzenesulfonamide

To a precooled solution of 3-nitrobenzene-1-sulfonyl chloride (5.0 g, 22.6 mmol) in DCM (100 mL) at 0° C. was added trietylamine (12.6 mL, 90.4 mmol) followed by the dropwise addition of 2-methoxyethylamine (3.9 mL, 45.1 mmol). The reaction mixture was stirred at room temperature for 2 hours, and diluted with water (50 mL). The organic layer was separated and the aqueous layer was extracted twice with DCM. The combined organic layers were washed with brine, dried over MgSO₄ and concentrated under reduced pressure to afford the titled compound (5.05 g, 86%) as a light yellow oil. ¹H NMR (600 MHz, CDCl₃): δ ppm 3.18 (t, J=7.5 Hz, 2H), 3.24 (s, 3H), 3.41 (t, J=7.5 Hz, 2H), 7.72 (dd, J=7.8 and 7.8 Hz, 1H), 8.17 (d, J=7.8 Hz, 1H), 8.40 (d, J=7.80 Hz, 1H), 8.69 (s, 1H). LCMS (Method A): 4.73 min [MH]⁺=259.5.

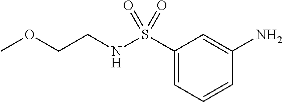

### Step 2: 3-Amino-N-(2-methoxyethyl)benzenesulfonamide

N-(2-methoxyethyl)-3-nitrobenzene sulfonamide (2.0 g, 7.68 mmol) was dissolved in MeOH (100 mL) and hydrogenated with an H-cube apparatus (Full H₂ mode, 40° C., 1 mL/min over 2 runs) using a Pt/C cartridge. The solvent was concentrated under reduced pressure to afford the titled compound (1.8 g, 100%) as a light yellow oil. Used without any further purification. LCMS (Method A): 3.87 min [MH]⁺=231.2.

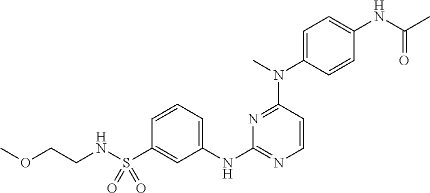 [SMILES: COCCNS(=O)(=O)c1cccc(Nc2nccc(N(C)c3ccc(NC(C)=O)cc3)n2)c1]

### Step 3: N-(4-((2-(3-(N-(2-Methoxyethyl)sulfamoyl)phenylamino)pyrimidin-4-yl)(methyl)amino)phenyl)acetamide

N-(4-((2-chloropyrimidin-4-yl)(methyl)amino)phenyl) acetamide (product of step 3 for the synthesis of Compound 5, 100 mg, 0.361 mmol) and 3-amino-N-(2-methoxyethyl)benzenesulfonamide (from step 2 above, 166 mg, 0.723 mmol) were dissolved in i-PrOH (5 mL) and concentrated HCl (5 drops) was added dropwise to the stirring solution. The reaction mixture was stirred at 80° C. for 3 hours and then cooled to room temperature. The off-white precipitate was collected by filtration, washed with EtOAc and dried under vacuum to afford the titled compound (90 mg, 53%) as a yellow solid. LCMS (Method A): 4.25 min [MH]⁺=471.8

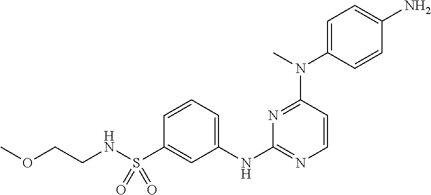 [SMILES: COCCNS(=O)(=O)c1cccc(Nc2nccc(N(C)c3ccc(N)cc3)n2)c1]

### Step 4: 3-(4-((4-Aminophenyl)(methyl)amino)pyrimidin-2-ylamino)-N-(2-methoxyethyl) benzenesulfonamide

N-(4-((2-(3-(N-(2-methoxyethyl)sulfamoyl)phenylamino) pyrimidin-4-yl)(methyl)amino) phenyl)acetamide (from step 3 above, 30 mg, 0.064 mmol) was dissolved in MeOH (1 mL). Acetyl chloride (0.45 mL, 0.64 mmol) was added dropwise and the reaction mixture was stirred at room temperature for 16 hours and concentrated in vacuo to afford the titled compound (25 mg, 89%) as a white solid. LCMS (Method A): 3.88 min [MH]⁺=429.4.

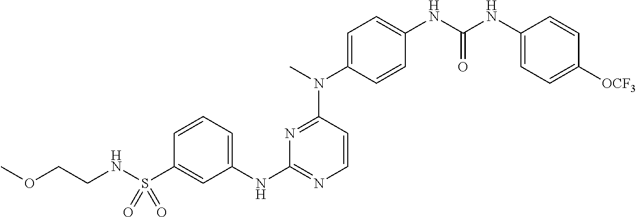 [SMILES: COCCNS(=O)(=O)c1cccc(Nc2nccc(N(C)c3ccc(NC(=O)Nc4ccc(OC(F)(F)F)cc4)cc3)n2)c1]

### Step 5: N-(2-Methoxyethyl)-3-(4-(methyl(4-(3-(4-(trifluoromethoxy)phenyl)-ureido) phenyl)amino)pyrimidin-2-ylamino)benzenesulfonamide

Following general procedure A with 3-(4-((4-Aminophenyl)(methyl)amino)pyrimidin-2-ylamino)-N-(2-methoxyethyl)benzenesulfonamide, the titled compound was obtained as a light yellow solid (4 mg, 10%). ¹H NMR (600 MHz, MeOD-d₄): δ ppm 3.05 (t, J=8.0 Hz, 2H), 3.24 (s, 3H), 3.37 (t, J=8.0 Hz, 2H), 3.50 (s, 3H), 5.86 (m, 1H), 7.20 (d, J=9.0 Hz, 1H), 7.23 (d, J=8.4 Hz, 1H), 7.42 (d, J=9.0 Hz, 2H), 7.53 (d, J=9.0 Hz, 2H), 7.56 (d, J=8.4 Hz, 2H), 7.70 (m, 1H), 7.78 (m, 1H), 8.56 (br s, 1H). LCMS (Method A): 4.75 min [MH]⁺=632.5.

Compound 24

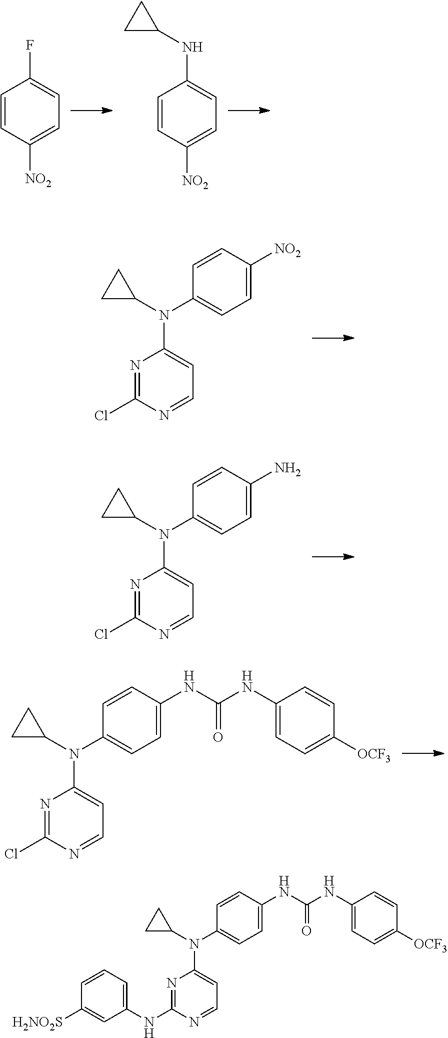 [SMILES: O=[N+]([O-])c1ccc(F)cc1 ; O=[N+]([O-])c1ccc(NC2CC2)cc1 ; O=[N+]([O-])c1ccc(N(c2ccnc(Cl)n2)C2CC2)cc1 ; Nc1ccc(N(c2ccnc(Cl)n2)C2CC2)cc1 ; O=C(Nc1ccc(OC(F)(F)F)cc1)Nc1ccc(N(c2ccnc(Cl)n2)C2CC2)cc1 ; NS(=O)(=O)c1cccc(Nc2nccc(N(c3ccc(NC(=O)Nc4ccc(OC(F)(F)F)cc4)cc3)C3CC3)n2)c1]

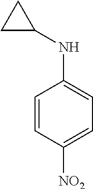 [SMILES: O=[N+]([O-])c1ccc(NC2CC2)cc1]

### Step 1: N-cyclopropyl-4-nitroaniline

To a solution of 4-fluoro-1-nitrobenzene (5.0 g, 35.4 mmol) in DMSO (15 mL) was added cyclopropanamine (4.0 g, 70.9 mmol) and potassium carbonate (9.8 g, 70.9 mmol). The reaction mixture was stirred at 70° C. for 16 h. The mixture was cooled and poured into water. The solid was filtered and dried to give N-cyclopropyl-4-nitroaniline (6.1 g, 97%) as a yellow solid. LCMS (Method B): 2.42 [MH]⁺=179.1.

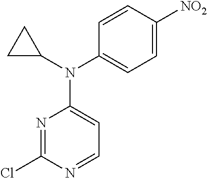 [SMILES: O=[N+]([O-])c1ccc(N(c2ccnc(Cl)n2)C2CC2)cc1]

### Step 2: 2-chloro-N-cyclopropyl-N-(4-nitrophenyl)pyrimidin-4-amine

The mixture of 2-chloro-N-cyclopropyl-N-(4-nitrophenyl)pyrimidin-4-amine (500 mg, 2.81 mmol), 2,4-dichloropyrimine (836 mg, 5.61 mmol), cesium carbonate (1.82 g, 5.61 mmol) in DMSO (10 mL) was stirred at 90° C. for 16 h. The reaction mixture was partitioned between ethylacetate and water. The aqueous phase was extracted with ethyl acetate. The combined organic phases were washed with brine, dried over sodium sulfate, concentrated and purified by column chromatography (petroleum ether/ethylacetate, 4:1) to give 2-chloro-N-cyclopropyl-N-(4-nitrophenyl)pyrimidin-4-amine (500 mg, 61%) as a yellow solid. LCMS (Method B): 2.57 [MH]⁺=291.1.

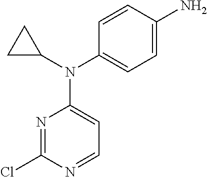 [SMILES: Nc1ccc(N(c2ccnc(Cl)n2)C2CC2)cc1]

### Step 3: N-1-(2-chloropyrimidin-4-yl)-N1-cyclopropylbenzene-1,4-diamine

To the mixture of 2-chloro-N-cyclopropyl-N-(4-nitrophenyl)pyrimidin-4-amine (500 mg, 1.72 mmol) in methanol (15 mL), zinc powder (1.12 g, 17.2 mmol) and NH₄Cl solution (10 mL) was added. The reaction mixture was stirred at 60° C. for 3 h. Methanol was removed and the mixture was partitioned between ethyl acetate and 1 M sodium hydroxide solution. The solid was filtered off. The organic layer phase was washed with brine, and dried. The solvent was removed to give N-1-(2-chloropyrimidin-4-yl)-N1-cyclopropylbenzene-1,4-diamine (440 mg, 98%) as light yellow solid. LCMS (Method B): 1.06 [MH]⁺=261.1.

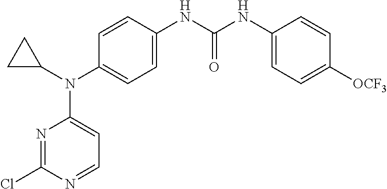 [SMILES: O=C(Nc1ccc(OC(F)(F)F)cc1)Nc1ccc(N(c2ccnc(Cl)n2)C2CC2)cc1]

### Step 4:1-(4-((2-chloropyrimidin-4-yl)(cyclopropyl)amino)phenyl)-3-(4-(trifluoromethoxy) phenyl)urea

General procedure A was followed with N-1-(2-chloropyrimidin-4-yl)-N1-cyclopropyl-benzene-1,4-diamine (440 mg, 1.69 mmol) and 1-isocyanato-4-(trifluoromethoxy)benzene (384 mg, 1.69 mmol) to afford and 4-1-(4-((2-chloropyrimidin-4-yl)(cyclopropyl)amino)phenyl)-3-(4-(trifluoromethoxy)-phenyl)urea (500 mg, 61%). LCMS (Method B): 3.09 [MH]⁺=464.1.

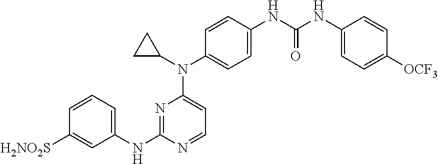 [SMILES: NS(=O)(=O)c1cccc(Nc2nccc(N(c3ccc(NC(=O)Nc4ccc(OC(F)(F)F)cc4)cc3)C3CC3)n2)c1]

### Step 5: 3-((4-(Cyclopropyl(4-(3-(4-(trifluoromethoxy)phenyl)ureido)-phenyl)amino)-pyrimidin-2-yl)amino)benzenesulfonamide

Following general procedure C using 1-(4-((2-chloropyrimidin-4-yl)(cyclopropyl)amino)phenyl)-3-(4-(trifluoromethoxy)phenyl)urea (93 mg, 0.20 mmol) and 3-aminophenylsulfamide (35 mg, 0.20 mmol), 3-((4-(Cyclopropyl(4-(3-(4-(trifluoro-methoxy)phenyl)ureido)phenyl)amino)pyrimidin-2-yl)amino)benzenesulfonamide (40 mg, 33%) was obtained as a yellow solid after purification by prep TLC (DCM/MeOH, 10:1). ¹H NMR (400 MHz, DMSO-d₆): δ ppm 0.56 (m, 2H), 0.97 (m, 2H), 3.22 (m, 1H), 6.17 (br s, 1H), 7.18 (d, J=8.4 Hz, 2H), 7.32 (m, 6H), 7.61 (m, 4H), 7.91 (d, J=8.0 Hz, 1H), 8.02 (d, J=6.0 Hz, 1H), 8.17 (s, 1H), 8.99 (s, 1H), 9.07 (s, 1H), 9.59 (s, 1H).

LCMS (Method B): 2.52 min [MH]⁺=600.2, [MNa]⁺=622.2.

Compound 25

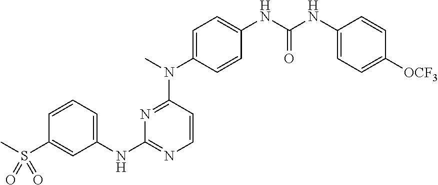 [SMILES: CN(c1ccc(NC(=O)Nc2ccc(OC(F)(F)F)cc2)cc1)c1ccnc(Nc2cccc(S(C)(=O)=O)c2)n1]

### 1-(4-(Methyl(2-((3-(methylsulfonyl)phenyl)amino)pyrimidin-4-yl)amino)phenyl)-3-(4-(trifluoromethoxy)phenyl)urea

Following general procedure C using intermediate C (100 mg, 0.23 mmol) and 3-methylsulfonyl aniline (47 mg, 0.23 mmol), 1-(4-(Methyl(2-((3-(methylsulfonyl) phenyl)amino)pyrimidin-4-yl)amino)phenyl)-3-(4-(trifluoromethoxy)phenyl)urea (80 mg, 61%) was obtained as a white solid after purification by column chromatography (DCM/MeOH, 100:1 to 60:1). ¹H NMR (400 MHz, DMSO-d₆): δ ppm 3.17 (s, 3H), 3.45 (s, 3H), 5.82 (d, J=6 Hz, 1H), 7.32 (m, 4H), 7.52 (m, 2H), 7.60 (m, 4H), 7.91 (m, 2H), 8.70 (s, 1H), 8.94 (s, 1H), 8.98 (s, 1H), 9.67 (s, 1H). LCMS (Method B): 2.57 min [MH]⁺=573.2, [MNa]⁺=595.2.

Compound 26

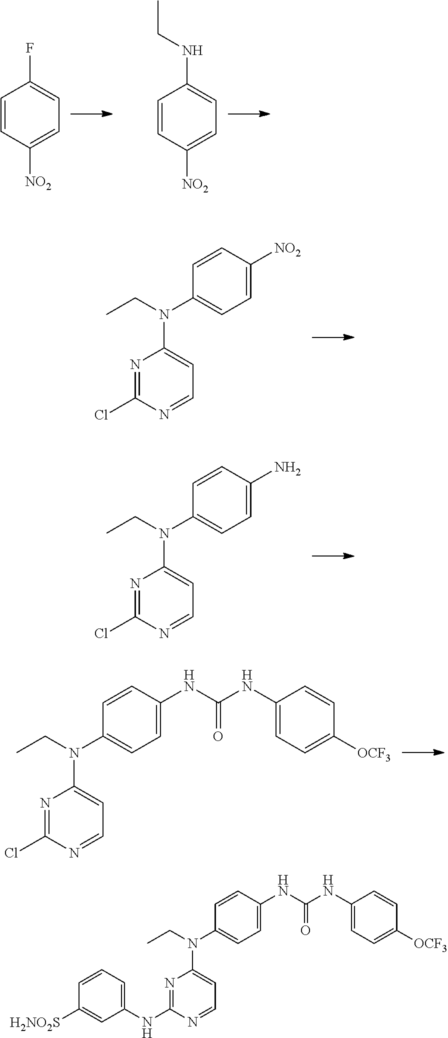 [SMILES: O=[N+]([O-])c1ccc(F)cc1 ; CCNc1ccc([N+](=O)[O-])cc1 ; CCN(c1ccc([N+](=O)[O-])cc1)c1ccnc(Cl)n1 ; CCN(c1ccc(N)cc1)c1ccnc(Cl)n1 ; CCN(c1ccc(NC(=O)Nc2ccc(OC(F)(F)F)cc2)cc1)c1ccnc(Cl)n1 ; CCN(c1ccc(NC(=O)Nc2ccc(OC(F)(F)F)cc2)cc1)c1ccnc(Nc2cccc(S(N)(=O)=O)c2)n1]

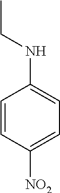

### Step 1: N-ethyl-4-nitrobenzenamine

In a bottom flask, 1-fluoro-4-nitrobenzene (3 g, 21.3 mmol), EtNH₂.HCl 3.46 g, 42.5 mmol), K₂CO₃ (6.46 g, 46.3 mmol) were dissolved in DMSO (20 mL). The reaction mixture was stirred at 85° C. overnight. The reaction mixture was poured into water (200 mL) and the mixture was stirred for 0.5 hour. The solid was collected by filtration, dried to give N-ethyl-4-nitrobenzenamine (2.9 g, 82%) as a yellow solid. LCMS (acidic 5 min): 2.25 [M+H]⁺=167.1.

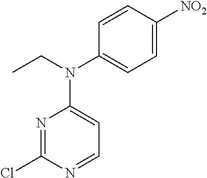 [SMILES: CCN(c1ccc([N+](=O)[O-])cc1)c1ccnc(Cl)n1]

### Step 2: 2-chloro-N-ethyl-N-(4-nitrophenyl)pyrimidin-4-amine

N-ethyl-4-nitrobenzenamine (1 g, 6.02 mmol), 2,4-dichloropyrimidine (896 mg, 6.02 mmol) and Cs₂CO₃ (3.9 g, 12.04 mmol) were dissolved in DMSO (15 mL). The reaction mixture was heated at 85° C. overnight. The reaction mixture was poured into water (70 mL), and the resulting mixture was extracted three times with EtOAc. The combined organic layer was washed three times with water and dried over Na₂SO₄. The solvent was removed to give 2-chloro-N-ethyl-N-(4-nitrophenyl)pyrimidin-4-amine (1 g, 60%) as a yellow solid which was used directly in next step. LCMS (acidic 5 min): 2.60 [M+H]⁺=279.0.

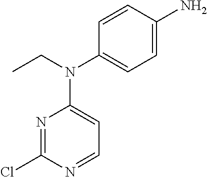 [SMILES: CCN(c1ccc(N)cc1)c1ccnc(Cl)n1]

### Step 3: N1-(2-chloropyrimidin-4-yl)-N1-ethylbenzene-1,4-diamine

2-chloro-N-ethyl-N-(4-nitrophenyl)pyrimidin-4-amine (1 g, 3.59 mmol) was dissolved in methanol (15 mL) and saturated NH₄Cl solution (15 mL). Zinc (powder, 2.13 g, 35.9 mmol) was added. The reaction mixture was stirred at room temperature overnight. The organic layer was removed under reduced pressure. The mixture was extracted three times with EtOAc (50 mL), and the combined organic layers were washed with aqueous NaCl (30 mL) and dried over Na₂SO₄. The solvent was removed under reduced pressure and the crude residue was purified by column chromatography (DCM/methanol, 100 to 100:1) to give N1-(2-chloropyrimidin-4-yl)-N1-ethylbenzene-1,4-diamine (200 mg, 23%) as a yellow solid. LCMS (acidic 5 min): 2.08 [M+H]⁺=249.0.

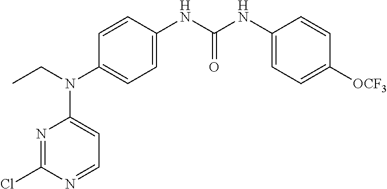 [SMILES: CCN(c1ccc(NC(=O)Nc2ccc(OC(F)(F)F)cc2)cc1)c1ccnc(Cl)n1]

### Step 4: 1-(4-((2-chloropyrimidin-4-yl)(ethyl)amino)phenyl)-3-(4-(trifluoro-methoxy)phenyl)urea

1-(4-((2-chloropyrimidin-4-yl)(ethyl)amino)phenyl)-3-(4-(trifluoromethoxy)-phenyl)urea was obtained using general procedure A with N1-(2-chloropyrimidin-4-yl)-N1-ethylbenzene-1,4-diamine and 4-trifluorophenyl-isocyanate. LCMS (acidic 5 min): 3.23 min [MH]⁺=452.1

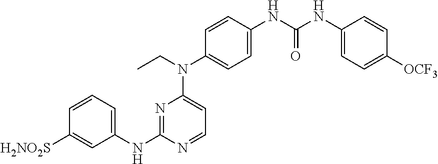 [SMILES: CCN(c1ccc(NC(=O)Nc2ccc(OC(F)(F)F)cc2)cc1)c1ccnc(Nc2cccc(S(N)(=O)=O)c2)n1]

### Step 5: 3-((4-(ethyl(4-(3-(4-(trifluoromethoxy)phenyl)ureido)phenyl)-amino)pyrimidin-2-yl)amino)benzenesulfonamide

Following general procedure C with 1-(4-((2-chloropyrimidin-4-yl)(ethyl)amino)phenyl)-3-(4-(trifluoromethoxy)phenyl)urea (220 mg, 0.487 mmol) and 3-aminobenzene sulfonamide (84 mg, 0.487 mmol, 3-((4-(ethyl(4-(3-(4-(trifluoromethoxy) phenyl)ureido)phenyl)amino)pyrimidin-2-yl)amino)benzenesulfonamide (68 mg, 24%) was obtained as a white solid after purification by column chromatography (DCM/MeOH, 100:0 to 97.5/2.5). ¹H NMR (400 MHz, DMSO-d₆): δ ppm 1.19 (t, J=7.2 Hz, 3H), 4.00 (t, J=7.2 Hz, 2H), 5.65 (d, J=6.0 Hz, 1H), 7.43 (m, 8H), 7.60 (m, 4H), 7.86 (m, 2H), 8.51 (s, 1H), 8.91 (s, 1H), 8.95 (s, 1H), 9.50 (s, 1H). LCMS (acidic 5 min): 2.50 min [MH]⁺=588.2.

Compound 27

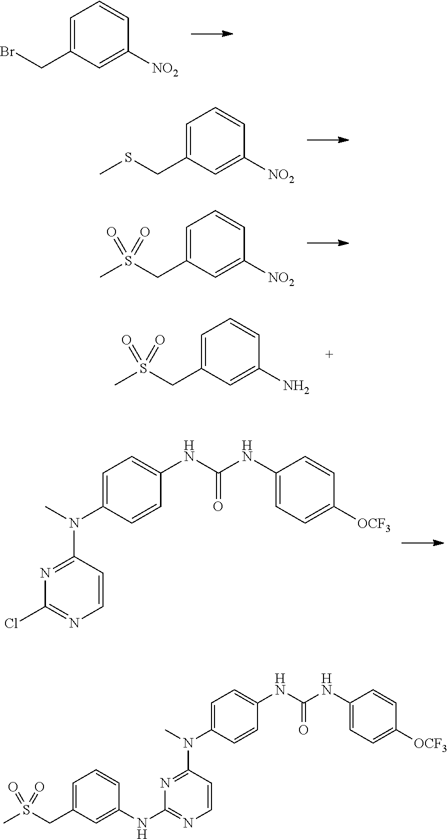

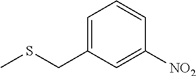

### Step 1: Methyl(3-nitrobenzyl)sulfane

A solution of 3-Nitrobenzyl bromide (3.72 g, 17.2 mmol) in ethanol (50 mL) was cooled to 0° C. Sodium thiomethoxide (1.45 g, 20.7 mmol) was added and the reaction mixture was stirred at room temperature for 3 hours. The reaction mixture was concentrated under reduced pressure, and partitioned between water and ethylacetate. The organic layer was separated and dried over sodium sulfate to give methyl(3-nitrobenzyl)sulfane (contaminated with about 26% of 1-(ethoxymethyl)-3-nitrobenzene, 3.22 g, 26%) as yellow oil. LCMS (Method B): 2.48 min [MNa]⁺=206.0.

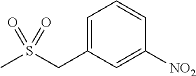

### Step 2: 1-((methylsulfonyl)methyl)-3-nitrobenzene

Methyl(3-nitrobenzyl)sulfane (70%, 3.22 g, 5.27 mmol of pure compound) was dissolved in DMF (50 mL). Oxone (9.72 g, 15.8 mmol) was added and the reaction was stirred at rt for 3 days. The resulting mixture was poured into water, and then ethylacetate was added. The organic layer was separated and washed with water and brine, dried over sodium sulfate and concentrated to give a residue which was purified by column chromatography (petroleum ether/ethylacetate, 5:1 to 1:1) to give 1-((methylsulfonyl)methyl)-3-nitrobenzene (940 mg, 29%) as a white solid. LCMS (Method B): 0.51 min [MNa]⁺=238.0.

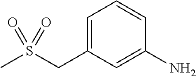

### Step 3: 3-((methylsulfonyl)methyl)aniline

1-((Methylsulfonyl)methyl)-3-nitrobenzene (50 mg, 0.23 mmol) was dissolved in ethylacetate (3 mL) and Pd/C (10%, 7 mg) was added. The reaction mixture was stirred under hydrogen at room temperature overnight. The Pd/C was filtered off and the filtrate was concentrated to give 3-((methylsulfonyl)methyl)aniline (46 mg, quantitative) as a grey solid used directly in the next step. LCMS (Method B): 0.26 min [MH]⁺=186.1.

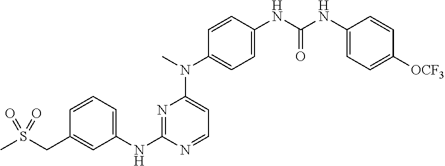 [SMILES: CN(c1ccc(NC(=O)Nc2ccc(OC(F)(F)F)cc2)cc1)c1ccnc(Nc2cccc(CS(C)(=O)=O)c2)n1]

### Step 4: 1-(4-(Methyl(2-((3-((methylsulfonyl)methyl)phenyl)amino)pyrimidin-4-yl)amino) phenyl)-3-(4-(trifluoromethoxy)phenyl)urea

Following general procedure C using intermediate C (108.7 mg, 0.248 mmol) and (methylsulfonyl)methyl)aniline (46 mg, 0.248 mmol), 1-(4-(Methyl(2-((3-((methylsulfonyl)methyl)phenyl)amino)pyrimidin-4-yl)amino)phenyl)-3-(4-(trifluoro-methoxy)phenyl)urea (57.7 mg, 40%) was obtained as a white solid after purified by column chromatography (DCM/MeOH, 20:1). ¹H NMR (400 MHz, DMSO-d₆): δ ppm 2.92 (s, 3H), 3.42 (s, 3H), 4.38 (s, 2H), 5.81 (d, J=6 Hz, 1H), 6.96 (d, J=8.0 Hz, 1H), 7.31 (m, 5H), 7.59 (m, 4H), 7.62 (d, J=8 Hz, 1H), 7.89 (m, 2H), 8.89 (s, 1H), 8.95 (s, 1H), 9.29 (m, 1H). LCMS (Method B): 2.55 min [MH]⁺=587.2.

Expression Constructs

cDNAs encoding mouse (residues 1-464) or human (residues 1-471) MLKL were synthesized to eliminate several restriction sites by silent substitutions (DNA2.0, CA). MLKL-encoding cDNAs were ligated into the doxycycline-inducible, puromycin selectable vector, pF TRE3G PGK puro, as described in Moujalled D M, et al. (2014), Cell Death Dis 5:e1086; Moujalled D M, et al. (2013) Cell Death Dis 4:e465; and Murphy J M, et al. (2013), Immunity 39(3):443-453. Sequences were verified by Sanger sequencing (Micromon DNA Sequencing Facility, VIC, Australia or by DNA2.0).

Lentiviral particles were produced by transfecting HEK293T cells seeded in 10 cm dishes with 1.2 μg of vector DNA together with two helper plasmids (0.8 μg of pVSVg and 2 μg of pCMV ΔR8.2) as described in Vince J E, et al. (2007), Cell 131(4):682-693. Viral supernatants were used to infect target cells with transfected cells selected for and maintained in 5 μg/ml puromycin.

Reagents and Antibodies

Recombinant hTNF-Fc was produced in-house as described in Bossen C, et al. (2006), The Journal of biological chemistry 281(20): 13964-13971. Puromycin, Doxycycline and Necrostatin-1 were purchased from Sigma-Aldrich. The Smac mimetic, Compound A, has been described previously in Vince J E, et al. (2007), Cell 131(4):682-693. Q-VD-OPh-OPH was purchased from R&D systems. The monoclonal rat anti-mouse MLKL antibody (clone 3H1) was raised in-house by the Walter and Eliza Hall Institute Monoclonal Facility (now available from Millipore, cat. MABC604). Anti-β-actin antibody was purchased from Sigma Aldrich; Anti-VDAC1 (AB10527) was purchased from Millipore; anti-GAPDH from Cell Signaling Technologies; and anti-FLAG (M2) from Sigma. Primary antibodies were used to Western blot membranes bearing transferred proteins and detected using HRP-conjugated secondary antibodies purchased from GE Healthcare and Jackson Immunoresearch and the ECL detection method (Millipore).

Cell Lines and Cell Death Assays

Mouse dermal fibroblasts (MDFs) were isolated from three MlkI^{-/-} mice and three congenic wild type mice and then immortalized by SV40 large T antigen to generate three biologically independent cell lines, as described in Murphy J M, et al. (2013), Immunity 39(3):443-453. Immortalized MDFs were similarly prepared from three Ripk3^{-/-} mice and congenic wild type mice. MDFs and HEK293T were maintained in Dulbecco's modified Eagle's medium (DMEM) supplemented with 8-10% v/v fetal calf serum (FCS), and 5 μg/mL puromycin for lines stably transduced with inducible expression constructs for MLKL. U937 cells were cultured in RPMI1640 supplemented with 8% v/v FCS. Cell death assays were carried out in 24 well plates, seeding 1×10⁵ cells per well. Cells attached over 4 h in the presence of 10 ng/mL doxycycline or 20 ng/mL were then treated with assorted combinations of Necrostatin (50 μM) and Q-VD-OPh (5 μM) 30 min prior to addition of TNF (100 ng/mL) and Smac mimetic (500 nM). After 24 h for MDF cells or 48 h for U937 cells, cells were harvested and PI positive cells (1 μg/mL) quantified using a BD FACSCalibur flow cytometer.

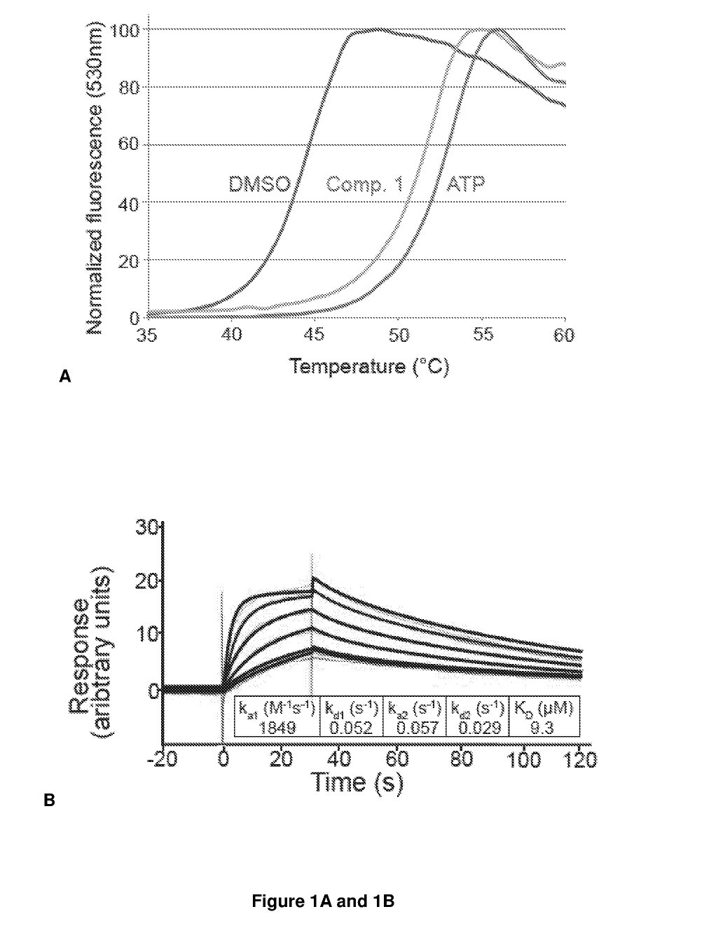

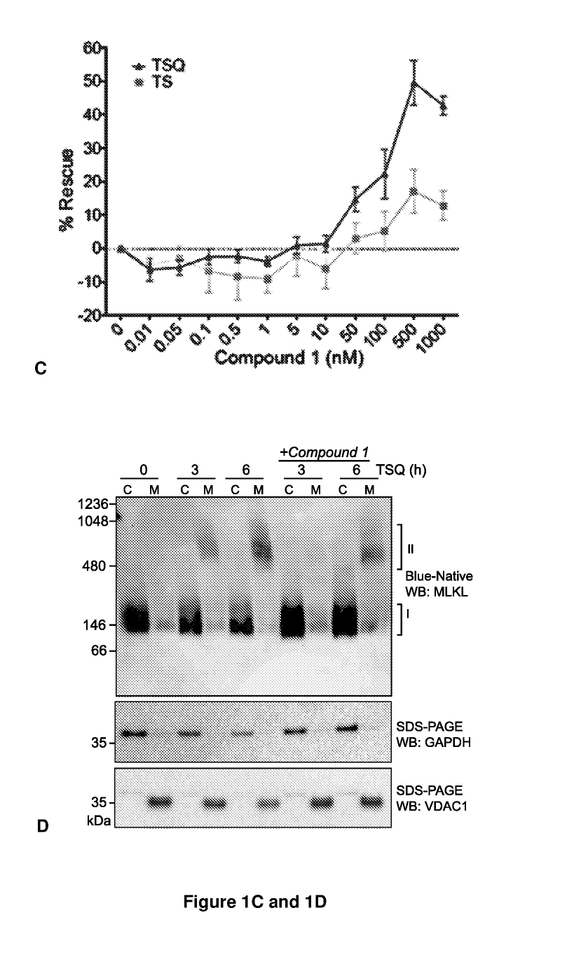

FIG. 1(C) demonstrates that compound 1 rescued ˜50% of wt MDFs from TSQ-induced necroptosis with an IC₅₀ of ˜500 nM.

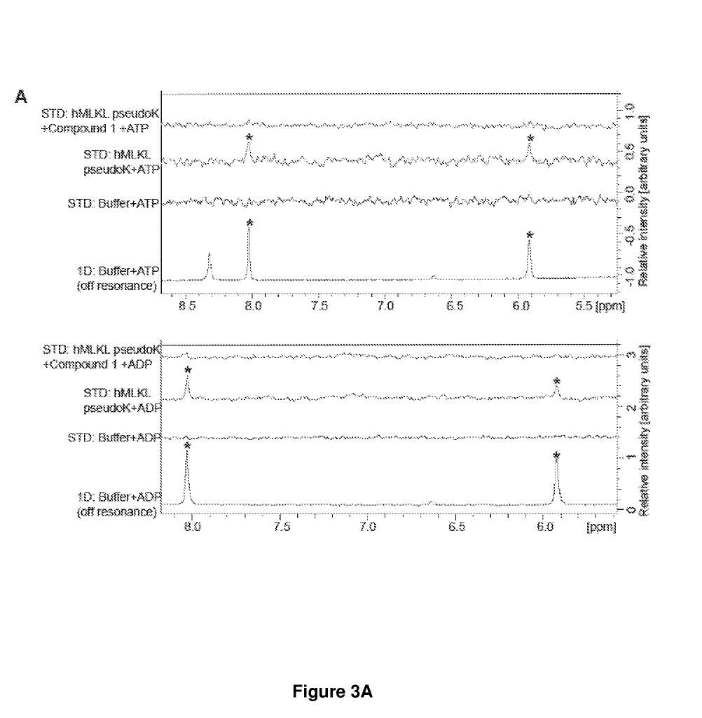

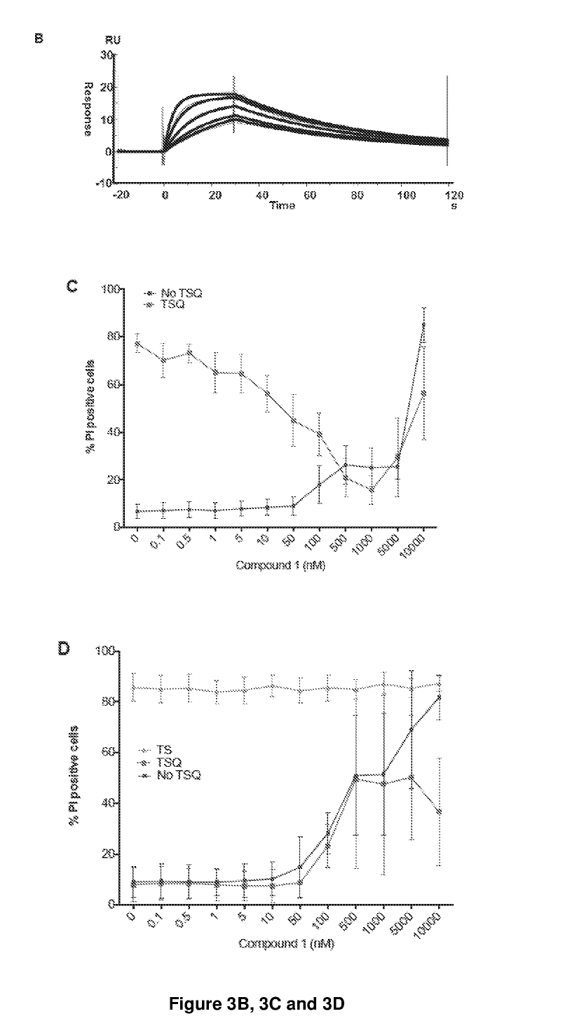

FIG. 3(C) demonstrates that compound 1 rescued ˜50% of wt U937 cells from TSQ-induced necroptosis with an IC₅₀ of 50-100 nM.

The ability of test compounds 1-4 to rescue wt U937 cells from TSQ-induced necroptosis is demonstrated in Table 1.

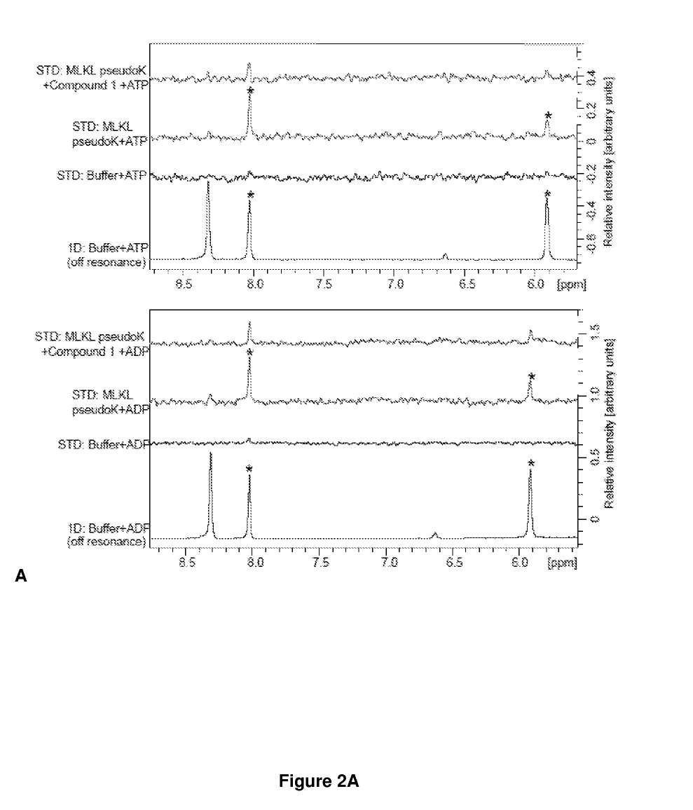

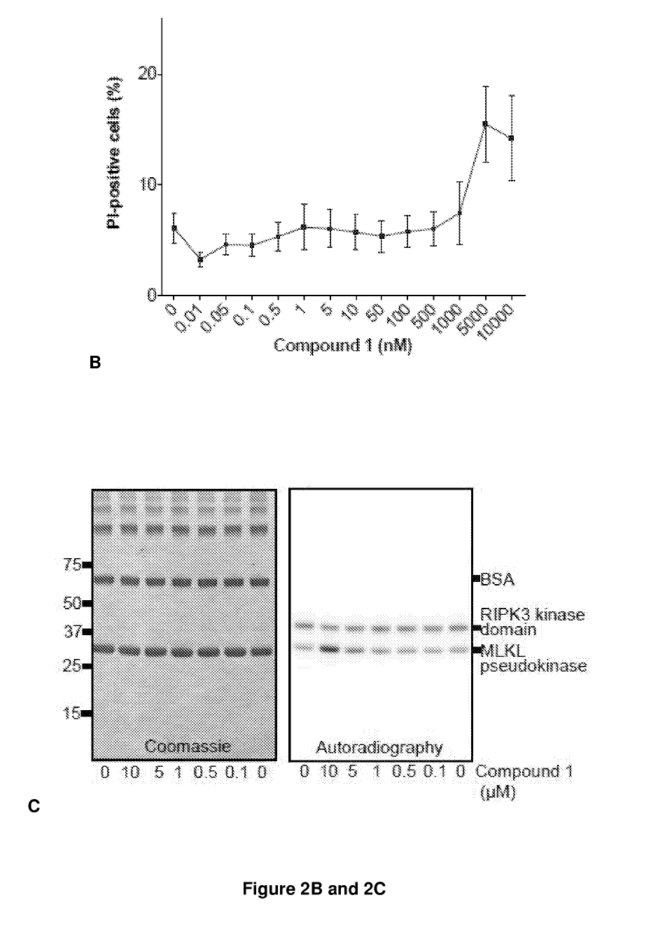

FIG. 2(B) demonstrates that the toxicity of compound 1 induces death of wild-type MDFs at concentrations ≧5 μM. Mean±SEM of triplicate experiments shown.

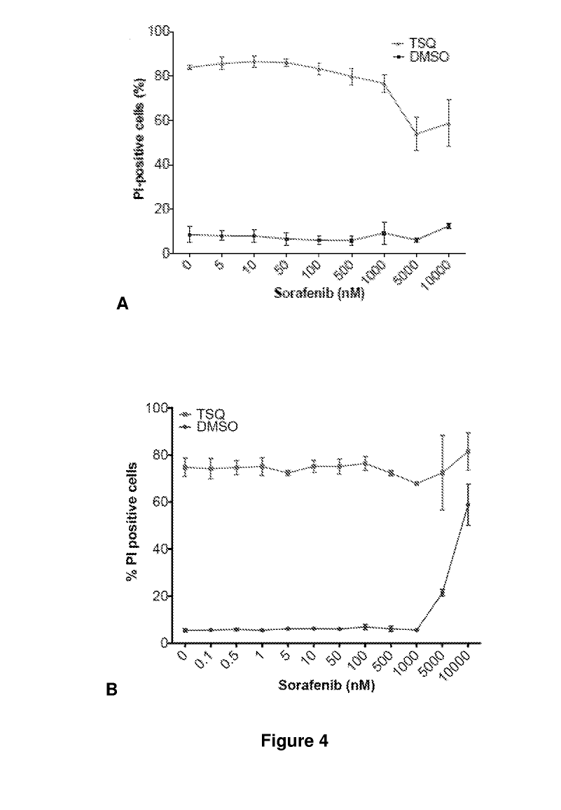

FIG. 3(D) demonstrates the toxicity of compound 1 in U937 cells gene-edited to delete MLKL. In the absence of MLKL, TSQ stimulated cells behaved equivalently to unstimulated cells. TS treatment illustrates that the cells have retained the capacity to undergo apoptotic death and this was unaffected by Compound 1 treatment. Data shown are mean±SD for 2 independent experiments. FIG. 4(A) demonstrates that sorafenib, a protein kinase inhibitor with a similar protein kinase target profile to compound 1, did not inhibit TSQ-induced necroptosis in wild type MDFs. Mean±SEM of triplicate experiments shown.

FIG. 4(B) demonstrates that sorafenib, a protein kinase inhibitor with a similar protein kinase target profile to compound 1, did not inhibit TSQ-induced necroptosis in wild type U937 cells. Mean±SEM of duplicate experiments shown.

Statistical Analyses

Error bars represent Mean+/−SD or SEM (specified in figure legends) of specified number of independent and or biological repeats, not technical replicates.

Fractionation and Blue Native PAGE

MDFs were seeded in 6 well plates (5×10⁵ per well) and allowed to attach overnight. Cells were stimulated with TSQ for up to 6 hours in the presence of 1 μM test compound or DMSO control. Cells were harvested by scraping, washed once in PBS and permeabilized in buffer (20 mM Hepes (pH 7.5), 100 mM KCl, 2.5 mM MgCl₂, and 100 mM sucrose) containing 0.025% digitonin (BIOSYNTH, Staad, Switzerland) and supplemented with EDTA-free Complete protease inhibitor cocktail (Roche), 2 μM N-ethyl maleimide, and phosphatase inhibitors (5 mM β-glycerophosphate, 1 mM Na Molybdate, 1 mM Na Pyrophosphate and 100 μM Na Fluoride). Permeabilization was confirmed by trypan blue uptake, and cytosolic and crude membrane fractions were separated by centrifugation at 11,000×g for 5 min. Digitonin was added to the cytoplasmic fraction to a final concentration of 1% and the crude membrane fraction was further solubilized in permeabilization buffer+1% digitonin and incubated on ice for 20 min. Crude membrane suspension was centrifuged at 11,000×g for 5 min and the supernatant loaded alongside cytoplasmic fraction on 4-16% Bis-Tris Native PAGE gel (LifeTechnologies). Following transfer of proteins to PVDF, PVDF was destained and the MLKL epitope was revealed by soaking the PVDF membrane in 6 M Guanidine Hydrochloride, 10 mM Tris-HCl pH 7.5 and 5 mM 2-Mercaptoethanol for 2 hours at room temperature. MLKL containing complexes were detected by anti-MLKL (3H1) Western blotting as described above.

FIG. 1(D) demonstrates that compound 1 retarded translocation to the membrane fraction in anti-MLKL blots of Blue-Native PAGE. Cytoplasmic and membrane fraction purity and protein abundance are illustrated by control blots for GADPH and VDAC1.

Recombinant Protein Expression and Purification

Recombinant mouse (residues 179-464) and human (190-471) MLKL pseudokinase domain bearing a conventional N-terminal His₆ tag as encoded by the pFastBac HTb vector or a modified 2×His₆ tag, MSHHHHHHGSAGSAKKKGSAGSAHHHHHHGSA, introduced into the pFastBac1 vector were expressed and purified from Sf21 insect cells according to established procedures as described in Murphy J M, et al. (2013), Immunity 39(3):443-453 and Murphy J M, et al. (2014), The Biochemical journal 457(2):323-334. Briefly, these proteins were purified from Sf21 lysates by Ni²⁺-affinity chromatography (Roche HisTag resin). The conventional His₆ tag was then cleaved by incubation with TEV protease for 1 hour at 25° C., before extensive dialysis, further Ni²⁺-chromatography to eliminate undigested protein and TEV protease followed by Superdex-200 gel filtration chromatography (GE Healthcare). Protein was eluted in 200 mM NaCl, 20 mM HEPES pH 7.5 for thermal stability shift assays or 100 mM NaCl, 20 mM HEPES pH 7.5 for NMR studies. Uncleaved 2×His₆-tagged pseudokinase Ni²⁺-eluate was concentrated by centrifugal ultrafiltration and subjected to Superdex-200 gel filtration chromatography (GE Healthcare) with elution in 200 mM NaCl, 20 mM Tris pH 8, 10% glycerol, 0.5 mM TCEP prior to use in Surface Plasmon Resonance experiments. Recombinant mRIPK3 kinase domain was expressed and purified from Sf21 cells as described previously in Murphy J M, et al. (2013), Immunity 39(3):443-453 and Cook W D, et al. (2014) Cell Death Diff. (in press).

Recombinant mouse MLKL(1-169) was prepared from E. coli (BL21 Codon Plus) using an established strategy as described in Hercus T R, et al. (2013), PloS one 8(8):e74376 and Murphy J M, et al. (2010), Growth factors 28(2):104-110. Briefly, a cDNA encoding mMLKL(1-169) was ligated inframe into the Kanamycin-selectable vector, pETNusH HTb, to enable expression as a fusion protein bearing an N-terminal, TEV protease cleavable NusA-His₆ tag. Bacteria were cultured in Super Broth containing 50 μg/mL Kanamycin at 37° C. until an OD₅₉₅ ˜0.6-0.8 was reached, before the temperature was lowered to 18° C. and, 20 min later, expression induced by addition of 1 mM IPTG. Cells were cultured for a further 16 h at 18° C., harvested by centrifugation, resuspended in 0.2M NaCl, 5 mM imidazole, 20 mM HEPES pH 7.5, 5 mM 2-mercaptoethanol supplemented with 1 mM PMSF, lysed by sonication and debris eliminated by centrifugation. The supernatant was clarified by syringe-driven 0.45 μM filtration and applied to a NiMAC cartridge (Novagen, Madison, Wis.) via peristaltic pump. Following washes with 7-10 column volumes of lysis buffer and lysis buffer containing 35 mM imidazole pH 7.5, NusA-His₆-mMLKL(1-169) was eluted in 0.2M NaCl, 250 mM imidazole, 20 mM HEPES pH 7.5, 5 mM 2-mercaptoethanol and incubated for 2 h at 20° C. with 0.5 mg TEV protease to cleave mMLKL(1-169) from the fusion tag. With the exception of the TEV protease cleavage step, all other purification steps were performed at 4° C. The cleavage reaction was then dialysed extensively against 0.2M NaCl, 20 mM HEPES pH 7.5 to eliminate imidazole before the dialysate was recovered and reapplied to a recharged NiMAC cartridge and washed with lysis buffer. The flow-through was concentrated by centrifugal ultrafiltration and applied to a Superdex-200, 24 mL gel filtration column (GE Healthcare) and eluted in 100 mM KCl, 10 mM Tris-HCl pH 8.0 for AUC studies.

Thermal Shift Assays to Screen for Small Molecule Interactors

Thermal shift assays were performed as described previously in Murphy J M, et al. (2013), Immunity 39(3):443-453, Murphy J M, et al. (2014), The Biochemical journal 457(2):323-334 and Murphy J M, et al. (2014), The Biochemical journal 457(3):369-377 using a Corbett Real Time PCR machine after diluting proteins to 2.6 μM in 150 mM NaCl, 20 mM Tris pH 8.0, 1 mM DTT in a total reaction volume of 25 μL. SYPRO Orange (Molecular Probes, Calif.) was used to detect protein thermal unfolding via fluorescence detected at 530 nm. ATP was added at 0.2 mM and was used as a positive control for ligand binding. Test compounds were added at 40 μM final concentration. A positive ΔTₘ value indicates that ligand binds the protein and confers protection from denaturation. Shown data are representative of three independent experiments.

FIG. 1(A) demonstrates the thermal shift assay for compound 1, confirming that compound 1 is a MLKL interactor.

Surface Plasmon Resonance (SPR) Binding Experiments

The kinetics of Compound 1 binding to mouse MLKL pseudokinase domain were determined by SPR on a Biacore T200 instrument (GE Healthcare). Double His-tagged MLKL and an unrelated negative control reference protein were immobilized on an NTA Capture chip charged with Ni²⁺ according to manufacturer's instructions. In some instances, double His-tagged proteins were captured via Ni²⁺/NTA chelation on a series S sensor chip containing carboxymethylated dextran surface pre-immobilized with nitrilotriacetic acid (NTA). The surface was then activated and enhanced with NHS/EDC mixture and His captured proteins covalently coupled to the surface as succinamide esters. Ethanolamine was injected later to block any unreacted esters. Unbound Ni²⁺ and non-covalently bound proteins were eluted by EDTA injections.

Typical immobilization levels were 2000-3000 Response Units (RU). Flow cell 1 was left blank as a reference surface. Immobilization experiments were carried out at 25° C. in a running buffer containing 20 mM HEPES (pH 8.0), 200 mM NaCl and 0.005% (v/v) surfactant P20.

Binding experiments were carried out in Running Buffer+2% v/v DMSO. Six Compound 1 concentrations ranging from 3.125 μM to 200 μM (in Running Buffer+2% v/v DMSO) were flowed over immobilized proteins at a flow rate of 100 μL/min, with an association phase of 30 s and dissociation phase of 90 s. Data were reduced, solvent corrected, and double referenced by Biacore T200 Evaluation Software. Data were fit globally to a two state kinetic interaction model and the K_{d} determined from the (k_{d}/kₐ) ratio. A 1:1 binding stoichiometry was inferred from the steady state binding curves and the maximum observed Response Unit (RU) levels.

FIG. 1(B) demonstrates that compound 1 binds the mouse MLKL(179-464) pseudokinase domain with a K_{d} value of 9.3 μM.

FIG. 3(B) demonstrates that compound 1 binds the human MLKL(190-471), pseudokinase domain with a K_{d} value of 4.4 μM.

Compounds 2-4 were also assayed by SPR to determine binding affinity. The binding affinity of compounds 2-4 is demonstrated in Table 1.

Saturation Transfer Difference (STD) NMR Spectroscopy

Nucleotides were dissolved in NMR buffer (20 mM HEPES, pH 7.5, 200 mM NaCl, 90% D₂O, 10% H₂O) at a final concentration of 200 μM in each sample of STD experiments. Three different samples were prepared for each nucleotide: (1) ATP or ADP with protein buffer added as equivalent volume of protein containing samples (2) ATP or ADP with MLKL pseudokinase domain at a final concentration of 2 μM (3) ATP or ADP with MLKL pseudokinase domain (2 μM) with test compound at 200 μM. NMR spectra were recorded at 283 K on a Bruker AVANCE Ultrashield 600 MHz spectrometer fitted with a Cryoprobe™. ¹H chemical shifts were referenced to the ¹H₂O signal at 4.70 ppm. Saturation of the protein resonances was achieved by a 4 s train of Gaussian pulses centred at −0.5 ppm. For the reference spectra, a similar saturation pulse was applied at a frequency centred 20,000 Hz off-resonance. A 15 ms T₂-spin-lock period was employed before acquisition to allow the residual protein signal to decay. NMR data were processed in TOPSPIN version 3.2 (Bruker BioSpin).

FIG. 2(A) demonstrates the STD NMR assay for compound 1. The STD NMR spectra showing nucleotide binding to mouse MLKL. The data show that compound 1 can compete with (i) ATP and (ii) ADP for binding to mouse MLKL pseudokinase domain. The low field region of the off resonance spectrum shows peaks detected for 200 μM ATP (i) or ADP (ii) in the absence of protein. Peaks marked with asterisks were observed in STD-NMR experiments performed on ATP (i) or ADP (ii) in the presence of 2 μM mouse MLKL(179-464), confirming nucleotide binding. These peaks were diminished in the presence of 200 μM compound 1, confirming that ATP and ADP are displaced from mouse MLKL(179-464) in the presence of compound 1.

FIG. 3(A) demonstrates the STD NMR assay for compound 1. The STD NMR spectra showing nucleotide binding to human MLKL. The data show that compound 1 can compete with (i) ATP and (ii) ADP for binding to human MLKL pseudokinase domain. The low field region of the off resonance spectrum shows peaks detected for 200 μM ATP (i) or ADP (ii) in the absence of protein. Peaks marked with asterisks were observed in STD-NMR experiments performed on ATP (i) or ADP (ii) in the presence of 2 μM human MLKL(190-471), confirming nucleotide binding. These peaks were diminished in the presence of 200 μM compound 1, confirming that ATP and ADP are displaced from human MLKL(190-471) in the presence of compound 1.

In Vitro Kinase Assays

In vitro kinase assays were performed as described previously in Murphy J M, et al. (2013), Immunity 39(3):443-453 and Cook W D, et al. (2014) Cell Death Diff. in press, but for the addition of either a DMSO control or up to 12.5 μM of test compound in 0.5% v/v final DMSO.

FIG. 2(C) demonstrates that compound 1 has no impact on recombinant RIPK3 kinase activity relative to a DMSO control (“0” lanes). Compound 1 concentrations ≧10 μM reproducibly led to enhanced phosphorylation of mouse MLKL(179-464). Experiment shown is representative of three independent assays. Left panel, dried Coomassie stained 4-12% Bis-Tris gel; right panel, autoradiograph of the same gel.

1.2 Results of Assays

The compounds described herein were assayed as described above. The results of the assays are set out in the table below.

TABLE 1
Table showing the binding affinity of test compounds as determined by SPR
and the ability of test compounds to rescue U937 cells from TSQ-induced necroptosis.

| | Affinity (K_{d}) | Inhibition of |
|---|---|---|
| | as determined | necroptosis |
| Compound | by SPR (μM) | (IC₅₀) (nM) |
| 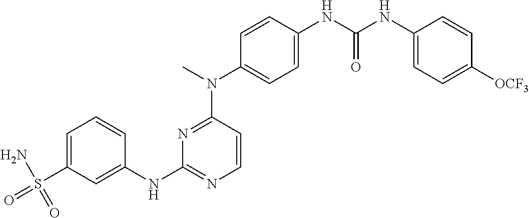 [SMILES: CN(c1ccc(NC(=O)Nc2ccc(OC(F)(F)F)cc2)cc1)c1ccnc(Nc2cccc(S(N)(=O)=O)c2)n1] 1 | 4.4 | 50-100 |
| 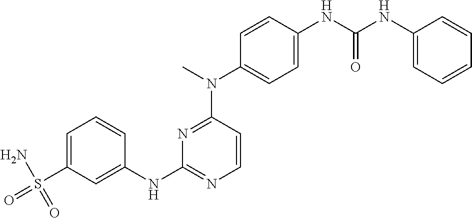 [SMILES: CN(c1ccc(NC(=O)Nc2ccccc2)cc1)c1ccnc(Nc2cccc(S(N)(=O)=O)c2)n1] 2 | 1.3 | 400-800 |
| 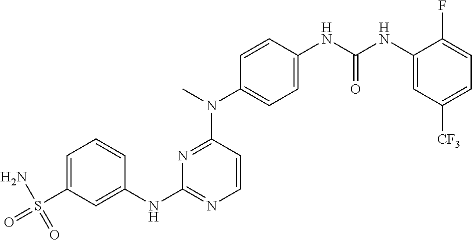 [SMILES: CN(c1ccc(NC(=O)Nc2cc(C(F)(F)F)ccc2F)cc1)c1ccnc(Nc2cccc(S(N)(=O)=O)c2)n1] 3 | 7 | 1-5 |
| 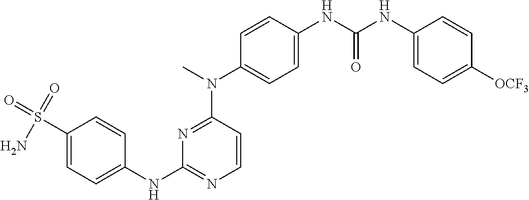 [SMILES: CN(c1ccc(NC(=O)Nc2ccc(OC(F)(F)F)cc2)cc1)c1ccnc(Nc2ccc(S(N)(=O)=O)cc2)n1] 4 | 4.7 | >1 μM |

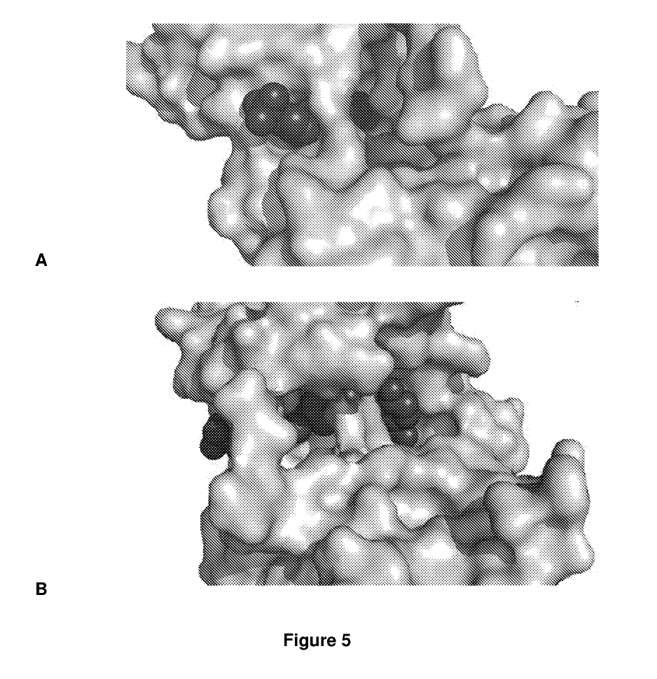

The binding of Compound 3, an analogue derived from compound 1, to the ATP-binding site of the MLKL protein is depicted in FIGS. 5 (A) and (B).

The effect of compound 1 binding to the MLKL pseudokinase domain on phosphorylation of MLKL by its upstream activator, RIPK3, was examined using in vitro kinase assays. It was determined that neither the catalytic activity of recombinant RIPK3 nor RIPK3-mediated phosphorylation of MLKL were inhibited by compound 1. On the contrary, in the presence of >5 μM compound 1, RIPK3-mediated phosphorylation of MLKL was enhanced. FIG. 2(D) demonstrates that compound 1 has no impact on recombinant RIPK3 kinase activity relative to a DMSO control (“0” lanes). Compound 1 concentrations 210 μM reproducibly led to enhanced phosphorylation of the MLKL(179-464). Experiment shown is representative of three independent assays. Left panel, dried Coomassie stained 4-12% Bis-Tris gel; right panel, autoradiograph of the same gel.

Screening Compounds for Inhibition of TSQ Induced Necroptosis, 96 Well Plate Format.

Cell Line ID:

U937 human histiocytic leukemia cell line.

Cell Concentration (Cells/Well):

35,000 per well in 120 μL of media, counted and plated immediately prior to addition of inhibitor and death stimuli. Final well volume of 150 μL after addition of compounds and death stimuli

Cell Growth Medium:

HTRPMI (WEHI Media kitchen, contains L-Glutamine and penicillin, streptomycin)—supplemented with 7.4% v/v FCS (Gibco, Precision Plus. Lot #1221437)

Incubation Time (Hours):

48 hours following addition of compounds and death stimuli

DMSO Final Concentration (% v/v): 0.2%

Compound Concentrations—Log Titrations:
10000 nM, 5000 nM, 1000 nM, 500 nM, 100 nM, 50 nM, 10 nM, 5 nM, nM, 0.5 nM, 0.1 nM

Compounds that are in the Death Stimulation Cocktail and their Final Concentrations:
hTNF-Fc (100 ng/ml)—produced by Silke lab, WEHI
Compound A (500 nM)—Smac mimetic, Tetralogic
Q-VD-OPh (10 μM)—MP Biomedicals

Analysis:
Cells treated with PI staining (1 μg/ml) and analysed by flow cytometry

Interpretation of Results:

Assay Involving the TSQ Cocktail (T:

TNF; S: Smac mimetic; Q: Q-VD-OPh): TSQ treatment ensures that cells specifically undergo necroptotic cell death. TNF activates the TNF receptor, Smac mimetic direct the signal away from proinflammatory signaling and toward the RIP1/RIP3-mediated cell death pathways, and Q-VD-OPh ensures that the apoptotic response is blocked leaving only the programmed necrosis response. The compounds' activity (solution in DMSO) tested in this TSQ-induced assay is evaluated by measuring their ability to block cell death as measured by flow cytometry after PI staining.

Counter Screen:

In parallel, all compounds are tested for their ability to affect cell viability. The same U937 cells are treated with compound in DMSO without the TSQ cocktail. This counter screen enables evaluation of off-target effects. In this case gain, cell viability is measured by flow cytometry after PI staining.

The results of the screening of the compounds described above are shown below in Table 2.

TABLE 2
Table showing the results of cell based assays and binding data
for compounds described above and comparative compounds.

| | | | | | | Off target effect | | | |
|---|---|---|---|---|---|---|---|---|---|
| | | Cell based assay | | | | (% cell death) | | | |
| Compound | | IC50 | Average | STDEV | SEM | at 500 nM | at 1 uM | at 5 uM | at 10 uM |
| 1 | = | 25.4495 | 69.4372 | 28.7130 | 6.5872 | <20 | <20 | 60 | 79 |
| 2 | | 122.7938 | | | | 24 | 23 | 32 | 87 |
| 3 | = | 3.3057 | 15.2998 | 12.4203 | 7.1709 | 40 | 72 | 86 | 84 |
| 4 | = | 1462.7054 | >5000 | | | | | | |
| 5 | ≧ | 10000 | >10000 | | | | | | |
| 6 | = | 18.9882 | 19.8981 | 1.2868 | | <20 | 40 | 86 | 89 |
| 7 | | | >10000 | | | | | | |
| 8 | = | 85.2349 | 89.0902 | 5.4523 | | | | | |
| 9 | = | 73.1013 | 42.0198 | 43.9559 | | <20 | <20 | 43 | 88 |
| 10 | = | 72.3531 | 45.6091 | 37.8217 | | <20 | <20 | 38 | 69 |
| 11 | = | 359.1179 | 359.1179 | 910.4298 | 779.6727 | <20 | <20 | <20 | 20 |
| 12 | = | 327.0835 | | | | 36 | 41 | 36 | 56 |
| 13 | = | 166.7859 | 148.7696 | 25.4790 | | 24 | 20 | 14 | 84 |
| 14 | = | 442.5674 | 341.5290 | 142.8899 | | <20 | <20 | <20 | 70 |
| 15 | = | 328.1019 | 304.4505 | 33.4480 | | <20 | <20 | <20 | 43 |
| 16 | = | 97.4625 | 87.3150 | 14.3506 | | <20 | <20 | <20 | 30 |
| 17 | = | 979.1332 | 1053.5487 | 105.2393 | | 22 | 21 | <20 | 39 |
| 18 | = | 91.7126 | 59.3997 | 45.6974 | | 27 | 29 | 94 | 93 |
| 19 | = | 114.2581 | 85.4834 | 40.6936 | | 28 | 30 | 26 | 70 |
| 20 | = | 142.6707 | 108.9614 | 47.6721 | | 24 | 29 | 33 | 80 |
| 21 | = | 72.6536 | 58.4430 | 20.0967 | | <20 | <20 | 51 | 79 |
| 22 | = | | 203.8038 | 35.9194 | | <20 | <20 | 35 | 80 |
| 23 | = | 39.0369 | 43.9489 | 6.9466 | | 21 | 21 | 22 | 64 |
| 24 | = | 143.6995 | 176.2074 | 45.9731 | | 20 | 21 | 66 | 80 |
| 25 | = | 21.6569 | 32.9203 | 15.9287 | | <20 | <20 | 43 | 86 |
| 26 | = | 126.4992 | 93.5984 | 46.5288 | | <20 | <20 | 43 | 69 |
| 27 | = | 191.3879 | 114.1738 | 109.1972 | | 24 | 20 | 33 | 84 |

Poly(I:C) Experiment

Transformed iBMDMs were split in 24 well plates (60.000 cells/well) and after 48 hrs treated with different concentrations of compound 1. After 0.5 hrs poly:IC (50 ug/ml) and zVAD (25 uM) were added. Cells were harvested after 48 hrs. Cell death was analysed via propidium iodide staining and flow cytometry.

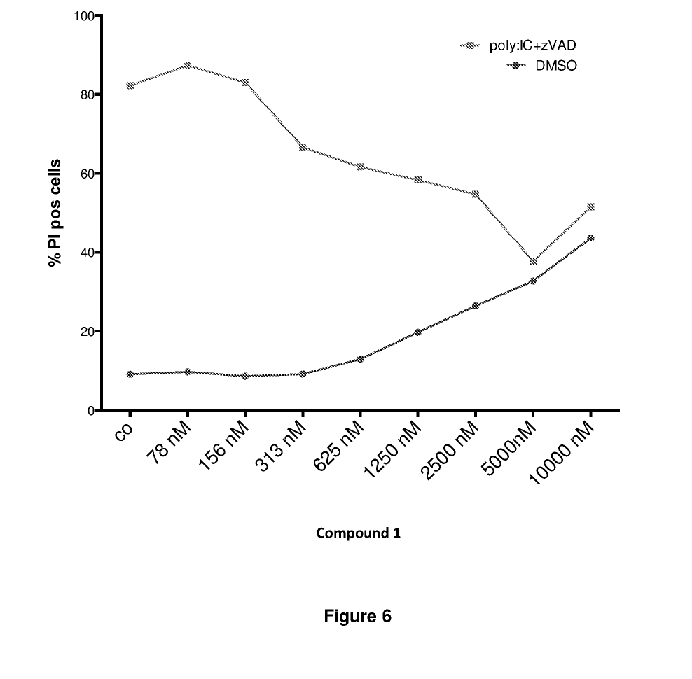

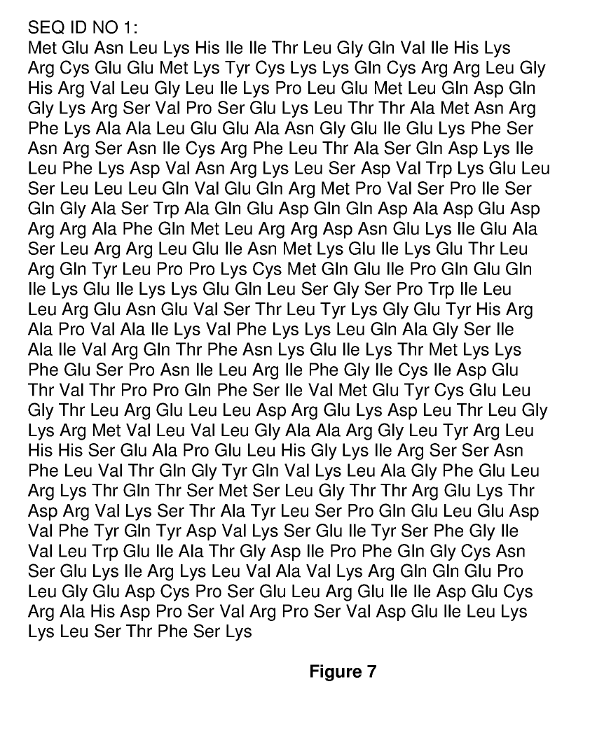

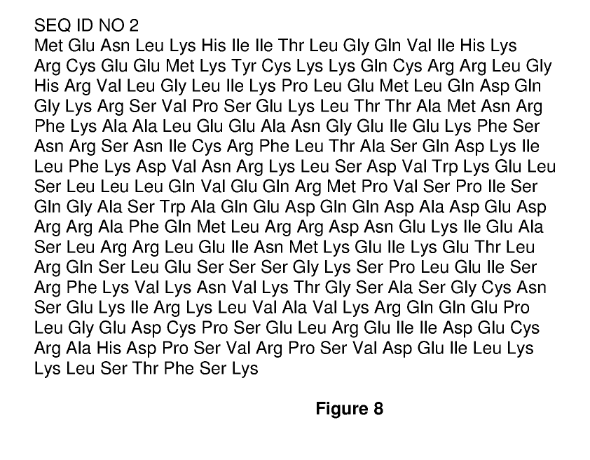

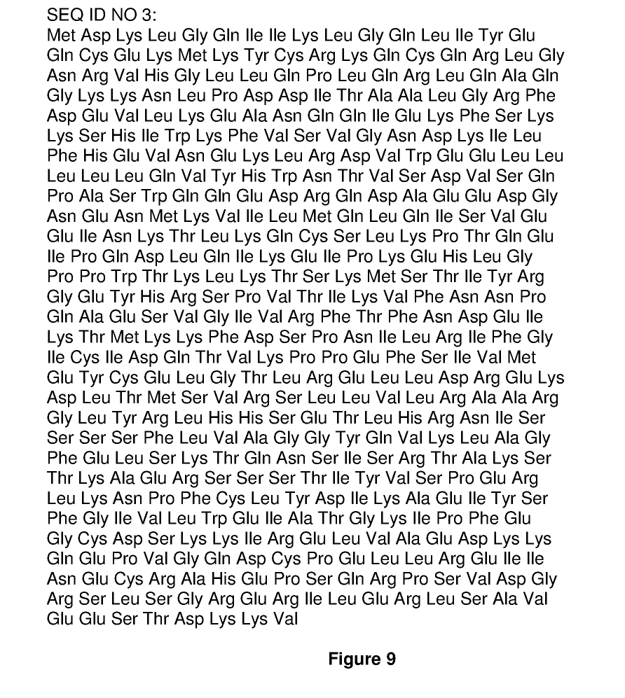

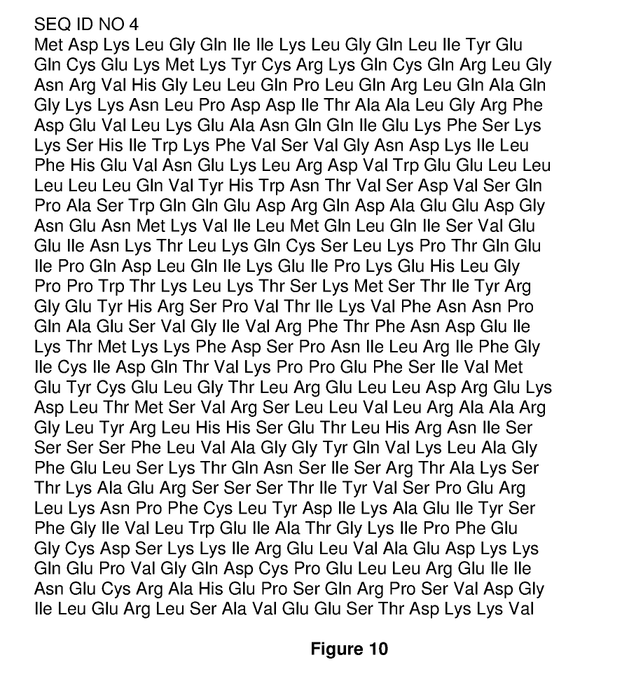

The results of FIG. 6 demonstrate that compound 1 inhibits Poly(I:C) and zVAD induced necroptosis. Poly(I:C) and zVAD induced cell death are known to occur independent of RIPK1 indicating that compound 1 inhibits necroptosis by targeting MLKL.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1-42. (canceled)

43. A method for inhibiting necroptosis in a subject in need thereof, the method comprising administering a therapeutically effective amount of a compound according to Formula (I): 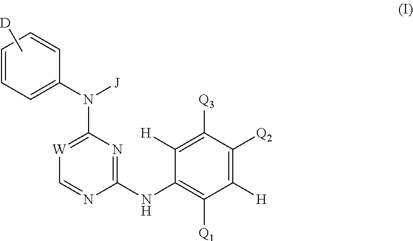
or a salt, solvate, or physiologically functional derivative thereof,
wherein:
W is N or C—R, wherein R is hydrogen, halogen, or cyano;
J is hydrogen, C₁-C₄ alkyl, C₁-C₄ haloalkyl, aralkyl, cyanoalkyl, —(CH₂)ₚC═CH(CH₂)ₜH, —(CH₂)ₚC≡C(CH₂)ₜH, or C₃-C₇ cycloalkyl;
p is 1, 2, or 3;
t is 0 or 1;
D is —N(H)(X);
X is the group defined by —(X₁)—(X₂)_{q}—(X₃) wherein
X₁ is C(O) or C(S) and q is 1, or
X₁ is —C(O) or —S(O)₂ and q is 0,
X₂ is N(H) or O, and
X₃ is alkyl, cycloalkyl, heterocyclyl, alkoxy, aryloxy, aralkoxy, aryl, aralkyl, or heteroaryl, or
alkyl, cycloalkyl, heterocyclyl, alkoxy, aryloxy, aralkoxy, aryl, aralkyl, or heteroaryl substituted with at least one group defined by —(X₄)_{z}—(X₅),
X₄ is C(H)₂ where z is 0, 1, 2, 3, or 4, and
X₅ is hydrogen, C₁-C₆ alkyl, C₁-C₆ haloalkyl, cycloalkyl, heterocyclyl, aryl, heteroaryl, alkoxy, haloalkoxy, hydroxy, aryloxy, aralkoxy, halo, —CN, —NR′R′, N(H)C(O)R″, N(H)C(O)OR″, N(H)C(O)NR′R′, N(H)S(O)₂R″, OR″, OC(O)RR″, C(O)R″, SR″, —S(O)R′″, —S(O)₂R′″, or —S(O)₂NR′R′, where,
R′ is hydrogen, alkyl, cycloalkyl, heterocyclyl, —OR¹, —SR¹, —S(O)₂R¹, —S(O)R¹, or C(O)R¹;
R″ is hydrogen, alkyl, cycloalkyl, heterocyclyl, —OR¹, —NR³R⁴, —S(O)₂R¹, —S(O)R¹ or C(O)R¹; and
R′″ is hydrogen, alkyl, cycloalkyl, heterocyclyl, —OR¹ or —NR³R⁴;
Q₁ is hydrogen, halogen, C₁-C₂ haloalkyl, C₁-C₂ alkyl, C₁-C₂ alkoxy, or C₁-C₂ haloalkoxy;
Q₂ is A¹ or A²;
Q₃ is A¹ when Q₂ is A² and Q₃ is A² when Q₂ is A¹;
wherein
A¹ is hydrogen, halogen, C₁-C₃ alkyl, C₁-C₃ haloalkyl, —OR¹, and
A² is the group defined by —(Z)ₘ—(Z¹)—(Z²), wherein
Z is CH₂ and m is 0, 1, 2, or 3, or
Z is NR² and m is 0 or 1, or
Z is oxygen and m is 0 or 1, or
Z is CH₂NR² and m is 0 or 1;
Z¹ is S(O)₂, S(O), or C(O); and
Z² is C₁-C₄ alkyl, cycloalkyl, heterocyclyl, NR³R⁴, aryl, arylamino, aralkyl, aralkoxy, or heteroaryl;
R¹ is hydrogen, alkyl, heterocyclyl, and —NR³R⁴;
R², R³, and R⁴ are each independently selected from hydrogen, hydroxy, alkoxy, aryloxy, aralkoxy, amino, alkylamino, arylamino, aralkylamino, C₁-C₄ alkyl, C₃-C₇ cycloalkyl, heterocyclyl, —S(O)₂R⁵, and —C(O)R⁵; and
R⁵ is C₁-C₄ alkyl, or C₃-C₇ cycloalkyl.

44. The method of claim 43, wherein the compound of Formula I is a compound according to Formula (II): 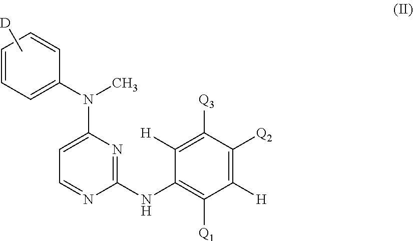
or a salt, solvate, or physiologically functional derivative thereof:
wherein:
D is —N(H)(X);
X is the group defined by —(X₁)—(X₂)_{q}—(X₃) wherein
X₁ is C(O) or C(S) and q is 1, or
X₁ is —C(O) or —S(O)₂ and q is 0,
X₂ is N(H) or O, and
X₃ is alkyl, cycloalkyl, heterocyclyl, alkoxy, aryloxy, aralkoxy, aryl, aralkyl, or heteroaryl, or
alkyl, cycloalkyl, heterocyclyl, alkoxy, aryloxy, aralkoxy, aryl, aralkyl, or heteroaryl substituted with at least one group defined by —(X₄)_{z}—(X₅),
X₄ is C(H)₂ where z is 0, 1, 2, 3, or 4, and
X₅ is hydrogen, C₁-C₆ alkyl, C₁-C₆ haloalkyl, cycloalkyl, heterocyclyl, aryl, heteroaryl, alkoxy, haloalkoxy, hydroxy, aryloxy, aralkoxy, halo, —CN, —NR′R′, N(H)C(O)R″, N(H)C(O)OR″, N(H)C(O)NR′R′, N(H)S(O)₂R″, OR″, OC(O)R″, C(O)R″, SR″, —S(O)R′″, —S(O)₂R′″, or —S(O)₂NR′R′, where,
R′ is hydrogen, alkyl, cycloalkyl, heterocyclyl, —OR¹, —SR¹, —S(O)₂R¹, —S(O)R¹, or C(O)R¹;
R″ is hydrogen, alkyl, cycloalkyl, heterocyclyl, —OR, —NR³R⁴, —S(O)₂R¹, —S(O)R¹, or C(O)R¹; and
R′″ is hydrogen, alkyl, cycloalkyl, heterocyclyl, —OR¹, or —NR³R⁴;
Q₁ is hydrogen, halogen, C₁-C₂ haloalkyl, C₁-C₂alkyl, C₁-C₂alkoxy, or C₁-C₂ haloalkoxy;
Q₂ is A¹ or A²;
Q₃ is A¹ when Q₂ is A² and Q₃ is A² when Q₂ is A¹;
wherein
A¹ is hydrogen, halogen, C₁-C₃ alkyl, C₁-C₃ haloalkyl, —OR¹, and
A² is the group defined by —(Z)ₘ—(Z¹)—(Z²),
wherein
Z is CH₂ and m is 0, 1, 2, or 3, or
Z is NR² and m is 0 or 1, or
Z is oxygen and m is 0 or 1, or
Z is CH₂NR² and m is 0 or 1;
Z¹ is S(O)₂, S(O), or C(O); and
Z² is C₁-C₄ alkyl, cycloalkyl, heterocyclyl, NR³R⁴, aryl, arylamino, aralkyl, aralkoxy, or heteroaryl;
R¹ is hydrogen, heterocyclyl, and —NR³R⁴;
R², R³, and R⁴ are each independently selected from hydrogen, hydroxy, alkoxy, aryloxy, aralkoxy, amino, alkylamino, arylamino, aralkylamino, C₁-C₄ alkyl, C₃-C₇ cycloalkyl, heterocyclyl, —S(O)₂R⁵, and —C(O)R⁵; and
R⁵ is C₁-C₄ alkyl, or C₃-C₇ cycloalkyl.

45. The method of claim 43 comprising administering a therapeutically effective amount of a compound according to Formula (I): 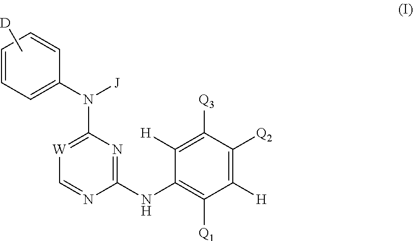
or a salt, solvate, or physiologically functional derivative thereof:
wherein:
W is N or C—R, wherein R is hydrogen, halogen, or cyano;
J is hydrogen, C₁-C₄alkyl, C₁-C₄ haloalkyl, aralkyl, cyanoalkyl, —(CH₂)ₚC═CH(CH₂)ₜH, —(CH₂)ₚC≡C(CH₂)ₜH, or C₃-C₇ cycloalkyl;
p is 1, 2, or 3;
t is 0 or 1;
D is 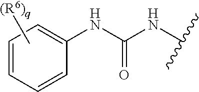
q is 1, 2, or 3;
Q₁ is hydrogen, halogen, C₁-C₂ haloalkyl, C₁-C₂ alkyl, C₁-C₂ alkoxy, or C₁-C₂ haloalkoxy;
Q₂ is A¹ or A²;
Q₃ is A¹ when Q₂ is A² and Q₃ is A² when Q₂ is A¹;
wherein
A¹ is hydrogen, halogen, C₁-C₃ alkyl, C₁-C₃ haloalkyl, —OR¹′ and
A² is the group defined by —(Z)ₘ—(Z¹)—(Z²),
wherein
Z is CH₂ and m is 0, 1, 2, or 3, or
Z is NR² and m is 0 or 1, or
Z is O and m is 0 or 1, or
Z is CH₂NR² and m is 0 or 1;
Z¹ is S(O)₂, S(O), or C(O); and
Z² is C₁-C₄ alkyl, cycloalkyl, heterocyclyl, NR³R⁴, aryl, arylamino, aralkyl, aralkoxy, or heteroaryl;
R², R³, and R⁴ are each independently selected from hydrogen, hydroxy, alkoxy, aryloxy, aralkoxy, amino, alkylamino, arylamino, aralkylamino, C₁-C₄ alkyl, C₃-C₇ cycloalkyl, heterocyclyl, —S(O)₂R⁵, and —C(O)R⁵;
R⁵ is C₁-C₆alkyl, or C₃-C₇ cycloalkyl; and
R⁶ is the group defined by —(X₄)_{z}—(X₅),
wherein
X₄ is C(H)₂ where z is 0, 1, 2, 3, or 4, and
X₅ is hydrogen, C₁-C₆ alkyl, C₁-C₆ haloalkyl, cycloalkyl, heterocyclyl, aryl, heteroaryl, alkoxy, haloalkoxy, hydroxy, aryloxy, aralkoxy, halo, CN, —NR⁷R⁷, —N(H)C(O)R⁷, —N(H)C(O)ORR⁷R⁷N(H)S(O)₂R⁷, N(H)S(O)₂NR⁷R⁷, —OC(O)R⁷, OC(O)NR⁷R⁷, —C(O)R⁷, —C(O)NR⁷R⁷, —SR⁷, —S(O)R⁷, —S(O)₂R⁷R⁷, or —S(O)₂NR⁷R⁷; and
R⁷ is hydrogen, C₁-C₆ alkyl, C₁-C₆ haloalkyl, cycloalkyl, heterocyclyl, alkylamino, alkoxy, aryloxy, arylamino, aralhylamino, aryl or heteroaryl.

46. The method of claim 43, wherein the compound of Formula I is a compound according to Formula (II): 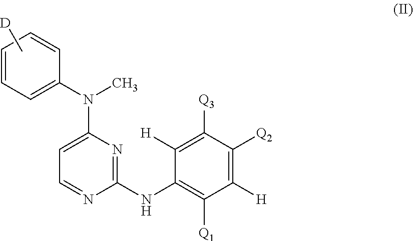
or a salt, solvate, or physiologically functional derivative thereof:
wherein:
D is 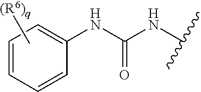
q is 1, 2, or 3;
Q₁ is hydrogen, halogen, C₁-C₂ haloalkyl, C₁-C₂ alkyl, C₁-C₂ alkoxy, or C₁-C₂ haloalkoxy;
Q₂ is A¹ or A²;
Q₃ is A when Q₂ is A² and Q₃ is A² when Q₂ is A¹; wherein
A¹ is hydrogen, halogen, C₁-C₃alkyl, C₁-C₃ haloalkyl, —OR¹, and
A² is the group defined by —(Z)ₘ—(Z¹)—(Z²),
wherein
Z is CH₂ and m is 0, 1, 2, or 3, or
Z is NR² and m is 0 or 1, or
Z is O and m is 0 or 1, or
Z is CH₂NR² and m is 0 or 1;
Z¹ is S(O)₂, S(O), or C(O); and
Z² is C₁-C₄ alkyl, cycloalkyl, heterocyclyl, NR³R⁴, aryl, arylamino, aralkyl, aralkoxy, or heteroaryl;
R², R³, and R⁴ are each independently selected from hydrogen, hydroxy, alkoxy, aryloxy, aralkoxy, amino, alkylamino, arylamino, aralkylamino, C₁-C₄ alkyl, C₃-C₇ cycloalkyl, heterocyclyl, —S(O)₂RS, and —C(O)R⁵;
R⁵ is C₁-C₆alkyl, or C₃-C₇ cycloalkyl; and
R⁶ is the group defined by —(X₄)_{z}—(X₅), wherein
X₄ is C(H)₂ where z is 0, 1, 2, 3, or 4, and
X₅ is hydrogen, C₁-C₆ alkyl, C₁-C₆ haloalkyl, cycloalkyl, heterocyclyl, aryl, heteroaryl, alkoxy, haloalkoxy, hydroxy, aryloxy, aralkoxy, halo, CN, —NR⁷R⁷, —N(H)C(O)R⁷, —N(H)C(O)OR⁷, —N(H)C(O)NR⁷R⁷, N(H)S(O)₂R⁷, N(H)S(O)₂NR⁷R⁷, —OC(O)R⁷, OC(O)NR⁷R⁷, —C(O)R⁷, —O)NR⁷R⁷, —SR⁷, —S(O)R⁷, —S(O)₂R⁷R⁷, or —S(O)₂NR⁷R⁷; and
R⁷ is hydrogen, C₁-C₆ alkyl, C₁-C₆ haloalkyl, cycloalkyl, heterocyclyl, alkylamino, alkoxy, aryloxy, aralkoxy, arylamino, aralkylamino, aryl or heteroaryl.

47. A method for inhibiting necroptosis in a subject in need thereof, the method comprising administering a therapeutically effective amount of a compound according to Formula (I): 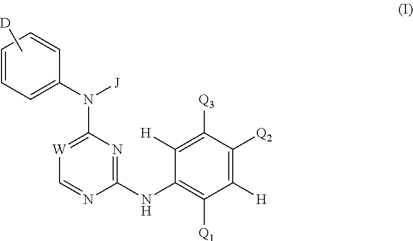
or a salt, solvate, or physiologically functional derivative thereof:
wherein:
W is N or C—R, wherein R is hydrogen, halogen, or cyano;
J is hydrogen, C₁-C₄alkyl, C₁-C₄ haloalkyl, aralkyl, cyanoalkyl, —(CH₂)ₚC═CH(CH₂)ₜH, —(CH₂)ₚC═C(CH₂)ₜH, or C₃-C₇ cycloalkyl;
p is 1, 2, or 3;
t is 0 or 1;
D is —N(R⁸)(X);
X is the group defined by —(X₁)—(X₂)_{q}—(X₃)
wherein
X₁ is C(O) or C(S) and q is 1, or
X₁ is —C(O) or —S(O)₂ and q is 0,
X₂ is N(H) or O, and
X₃ is alkyl, cycloalkyl, heterocyclyl, alkoxy, aryloxy, aralkoxy, aryl, aralkyl, or heteroaryl, or
alkyl, cycloalkyl, heterocyclyl, alkoxy, aryloxy, aralkoxy, aryl, aralkyl, or heteroaryl substituted with at least one group defined by —(X₄)_{z}—(X₅),
X₄ is C(H)₂ where z is 0, 1, 2, 3, or 4, and
X₅ is hydrogen, C₁-C₆ alkyl, C₁-C₆ haloalkyl, cycloalkyl, heterocyclyl, aryl, heteroaryl, alkoxy, haloalkoxy, hydroxy, aryloxy, aralkoxy, halo, —CN, —NR′R′, N(H)C(O)R″, N(H)C(O)OR″, N(H)C(O)NR′R′, N(H)S(O)₂R″, OR″, OC(O)R″, C(O)R″, SR″, —S(O)R′″, —S(O)₂R′″, or —S(O)₂NR′R′, where,
R′ is hydrogen, alkyl, cycloalkyl, heterocyclyl, —OR¹, —SR¹, —S(O)₂R¹, —S(O)R¹, or C(O)R¹;
R″ is hydrogen, alkyl, cycloalkyl, heterocyclyl, —OR¹, —NR³R⁴, —S(O)₂R¹, —S(O)R¹, or C(O)R¹; and
R′″ is hydrogen, alkyl, cycloalkyl, heterocyclyl, —OR¹, or —NR³R⁴;
Q₁ is hydrogen, halogen, C₁-C₂ haloalkyl, C₁-C₂ alkyl, C₁-C₂ alkoxy, or C₁-C₂ haloalkoxy;
Q₂ is A¹ or A²;
Q₃ is A¹ when Q₂ is A² and Q₃ is A² when Q₂ is A;
wherein
A¹ is hydrogen, halogen, C₁-C₃alkyl, C₁-C₃ haloalkyl, —OR¹, and
A² is the group defined by —(Z)ₘ—(Z¹)—(Z²),
wherein
Z is CH₂ and m is 0, 1, 2, or 3, or
Z is NR² and m is 0 or 1, or
Z is oxygen and m is 0 or 1, or
Z is CH₂NR² and m is 0 or 1;
Z¹ is S(O)₂, S(O), or C(O); and
Z² is C₁-C₄ alkyl, cycloalkyl, heterocyclyl, NR³R⁴, aryl, arylamino, aralkyl, aralkoxy, or heteroaryl;
R¹ is hydrogen, alkyl, heterocyclyl, and —NR³R⁴;
R², R³, and R⁴ are each independently selected from hydrogen, hydroxy, alkoxy, aryloxy, aralkoxy, amino, alkylamino, arylamino, aralkylamino, C₁-C₄ alkyl, C₃-C₇ cycloalkyl, heterocyclyl, —S(O)₂R⁵, and —C(O)R⁵;
R⁵ is C₁-C₄ alkyl, or C₃-C₇ cycloalkyl; and
R⁸ is hydrogen or C₁-C₃ alkyl.

48. The method of claim 43, wherein Q1, Q2 or Q3 is H₂NSO₂— or MeSO₂CH₂—.

49. The method of claim 43, wherein the subject has a disease selected from the group consisting of diseases of the bones, joints, connective tissue and cartilage, muscular diseases, skin diseases, cardiovascular diseases, circulatory diseases, hematological and vascular diseases, diseases of the lung, diseases of the gastro-intestinal tract, diseases of the liver, diseases of the pancreas, metabolic diseases, diseases of the kidneys, viral and bacterial infections, severe intoxications, degenerative diseases associated with the Acquired Immune Deficiency Syndrome (AIDS), disorders associated with aging, inflammatory diseases, auto-immune diseases, dental disorders, ophthalmic diseases or disorders, diseases of the audition tracts, diseases associated with mitochondria, and cancer and metastasis.

50-55. (canceled)

56. A compound selected from the group consisting of any one of compounds 5 to 10 and 12 to 26.

57. The method of claim 43, wherein the compound of Formula I is a compound according to Formula (III): 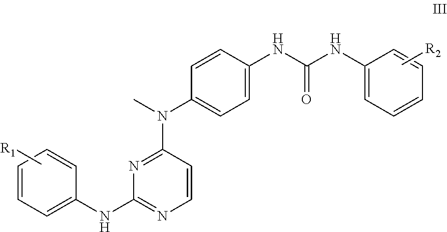
wherein: R₁ is selected from the group consisting of 3-MeSO₂CH₂—, 4-MeSO₂CH₂—, 3-H₂NSO₂— and 4-H₂NSO₂—; and
R₂ is 0-2 substituents independently selected from the group selected from the group consisting of OCF₃, CF₃, fluoro, chloro, bromo, iodo and COMe, or a pharmaceutically acceptable derivative, polymorph, salt or prodrug thereof.

58. The method of claim 57, wherein R₁ is 3-H₂NSO₂—.

59. The method of claim 57, wherein R₁ is 4-H₂NSO₂—.

60. The method of claim 57, wherein R₂ is 0 substituents.

61. The method of claim 57, wherein R₂ is 4-OCF₃.

62. The method of claim 57, wherein R₂ is 2 substituents, and wherein the 2 substituents are 3-CF₃ and 6-fluoro.

63. The method of claim 57, wherein the compound is selected from the group consisting of compounds 1 to 4

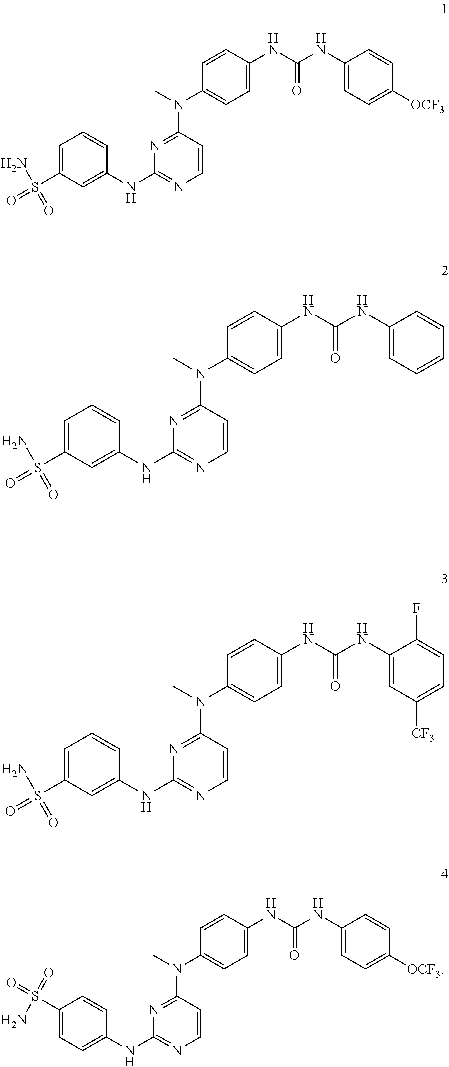 [SMILES: CN(c1ccc(NC(=O)Nc2ccc(OC(F)(F)F)cc2)cc1)c1ccnc(Nc2cccc(S(N)(=O)=O)c2)n1 ; CN(c1ccc(NC(=O)Nc2ccccc2)cc1)c1ccnc(Nc2cccc(S(N)(=O)=O)c2)n1 ; CN(c1ccc(NC(=O)Nc2cc(C(F)(F)F)ccc2F)cc1)c1ccnc(Nc2cccc(S(N)(=O)=O)c2)n1 ; CN(c1ccc(NC(=O)Nc2ccc(OC(F)(F)F)cc2)cc1)c1ccnc(Nc2ccc(S(N)(=O)=O)cc2)n1]

64. The method of claim 57, wherein the compound has the formula

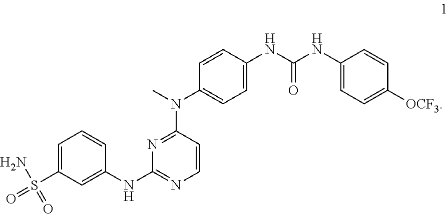 [SMILES: CN(c1ccc(NC(=O)Nc2ccc(OC(F)(F)F)cc2)cc1)c1ccnc(Nc2cccc(S(N)(=O)=O)c2)n1]

65. The method of claim 47, wherein the subject has a disease selected from the group consisting of diseases of the bones, joints, connective tissue and cartilage, muscular diseases, skin diseases, cardiovascular diseases, circulatory diseases, hematological and vascular diseases, diseases of the lung, diseases of the gastro-intestinal tract, diseases of the liver, diseases of the pancreas, metabolic diseases, diseases of the kidneys, viral and bacterial infections, severe intoxications, degenerative diseases associated with the Acquired Immune Deficiency Syndrome (AIDS), disorders associated with aging, inflammatory diseases, auto-immune diseases, dental disorders, ophthalmic diseases or disorders, diseases of the audition tracts, diseases associated with mitochondria, and cancer and metastasis.

66. The method of claim 47, wherein Q₁, Q₂ or Q₃ is H₂NSO₂— or MeSO₂CH₂—.


SEQUENCE LISTING (4 sequences)

SEQ ID NO: 1 - protein, 471 aa, Homo sapiens
MENLKHIITLGQVIHKRCEEMKYCKKQCRRLGHRVLGLIKPLEMLQDQGKRSVPSEKLTTAMNRFKAALEEANGEIEKFSNRSNICRFLTASQDKILFKDVNRKLSDVWKELSLLLQVEQRMPVSPISQGASWAQEDQQDADEDRRAFQMLRRDNEKIEASLRRLEINMKEIKETLRQYLPPKCMQEIPQEQIKEIKKEQLSGSPWILLRENEVSTLYKGEYHRAPVAIKVFKKLQAGSIAIVRQTFNKEIKTMKKFESPNILRIFGICIDETVTPPQFSIVMEYCELGTLRELLDREKDLTLGKRMVLVLGAARGLYRLHHSEAPELHGKIRSSNFLVTQGYQVKLAGFELRKTQTSMSLGTTREKTDRVKSTAYLSPQELEDVFYQYDVKSEIYSFGIVLWEIATGDIPFQGCNSEKIRKLVAVKRQQEPLGEDCPSELREIIDECRAHDPSVRPSVDEILKKLSTFSK

SEQ ID NO: 2 - protein, 263 aa, Homo sapiens
MENLKHIITLGQVIHKRCEEMKYCKKQCRRLGHRVLGLIKPLEMLQDQGKRSVPSEKLTTAMNRFKAALEEANGEIEKFSNRSNICRFLTASQDKILFKDVNRKLSDVWKELSLLLQVEQRMPVSPISQGASWAQEDQQDADEDRRAFQMLRRDNEKIEASLRRLEINMKEIKETLRQSLESSSGKSPLEISRFKVKNVKTGSASGCNSEKIRKLVAVKRQQEPLGEDCPSELREIIDECRAHDPSVRPSVDEILKKLSTFSK

SEQ ID NO: 3 - protein, 472 aa, Mus musculus
MDKLGQIIKLGQLIYEQCEKMKYCRKQCQRLGNRVHGLLQPLQRLQAQGKKNLPDDITAALGRFDEVLKEANQQIEKFSKKSHIWKFVSVGNDKILFHEVNEKLRDVWEELLLLLQVYHWNTVSDVSQPASWQQEDRQDAEEDGNENMKVILMQLQISVEEINKTLKQCSLKPTQEIPQDLQIKEIPKEHLGPPWTKLKTSKMSTIYRGEYHRSPVTIKVFNNPQAESVGIVRFTFNDEIKTMKKFDSPNILRIFGICIDQTVKPPEFSIVMEYCELGTLRELLDREKDLTMSVRSLLVLRAARGLYRLHHSETLHRNISSSSFLVAGGYQVKLAGFELSKTQNSISRTAKSTKAERSSSTIYVSPERLKNPFCLYDIKAEIYSFGIVLWEIATGKIPFEGCDSKKIRELVAEDKKQEPVGQDCPELLREIINECRAHEPSQRPSVDGRSLSGRERILERLSAVEESTDKKV

SEQ ID NO: 4 - protein, 464 aa, Mus musculus
MDKLGQIIKLGQLIYEQCEKMKYCRKQCQRLGNRVHGLLQPLQRLQAQGKKNLPDDITAALGRFDEVLKEANQQIEKFSKKSHIWKFVSVGNDKILFHEVNEKLRDVWEELLLLLQVYHWNTVSDVSQPASWQQEDRQDAEEDGNENMKVILMQLQISVEEINKTLKQCSLKPTQEIPQDLQIKEIPKEHLGPPWTKLKTSKMSTIYRGEYHRSPVTIKVFNNPQAESVGIVRFTFNDEIKTMKKFDSPNILRIFGICIDQTVKPPEFSIVMEYCELGTLRELLDREKDLTMSVRSLLVLRAARGLYRLHHSETLHRNISSSSFLVAGGYQVKLAGFELSKTQNSISRTAKSTKAERSSSTIYVSPERLKNPFCLYDIKAEIYSFGIVLWEIATGKIPFEGCDSKKIRELVAEDKKQEPVGQDCPELLREIINECRAHEPSQRPSVDGILERLSAVEESTDKKV